(19) United States
(12) Patent Application Publication
(10) Pub. No.: US 2022/0409692 A1
(43) Pub. Date: Dec. 29, 2022

(54) Systems Chemico-Pharmacology Drugs and Methods of Use

(71) Applicants: The Medical College of Wisconsin, Milwaukee, WI (US); Reneurogen LLC, Elm Grove, WI (US)

(72) Inventors: Kirkwood Arthur Pritchard, Jr., Elm Grove, WI (US); Dustin Paul Martin, Sturtevant, WI (US); Stephen Naylor, Elm Grove, WI (US)

(21) Appl. No.: 17/775,473

(22) PCT Filed: Nov. 11, 2020
(86) PCT No.: PCT/US2020/059991

§ 371 Date: May 9, 2022

Related U.S. Application Data
(60) Provisional application No. 62/933,656, filed on Nov. 11, 2019.

(51) Int. Cl. A61K 38/06

(52) U.S. Cl. CPC A61K 38/06

(57) ABSTRACT

As new class of drugs designated systems chemico-pharmacology drugs (SCPD) is disclosed. SCPDs work by targeting a druggable biomolecular site (the first target), resulting in a significant change in the properties of the first target. In the process, the SCPD itself is chemically modified. Subsequently, the resulting modified SCPD interacts with a second target, which is also modified in a manner that is beneficial for the patient.

### CROSS REFERENCE TO RELATED APPLICATIONS

This application claims the benefit of U.S. Provisional Patent Application No. 62/933,656 filed Nov. 11, 2019, which is incorporated herein by reference.

### STATEMENT REGARDING FEDERALLY SPONSORED RESEARCH OR DEVELOPMENT

This invention was made with government support under HL 128371 awarded by the U.S. National Institutes of Health. The government has certain rights in the invention.

### BACKGROUND OF THE INVENTION

It is well established that inflammation plays an important role in the pathogenesis of disease. Inflammation has been shown to be involved mechanistically in driving neurodegenerative disease, cardiovascular disease, airway disease, inflammatory bowel disease (IBD), diabetes and specific cancers.

One of the major factors promoting cellular injury during inflammation is oxidative stress. Oxidative stress is induced in cells by over production of reactive oxygen species (ROS), reactive nitrogen species (RNS) and/or an increase in heme peroxidase activity. During inflammation, cells produce highly reactive free radicals (e.g., superoxide anion, hydroxyl radical, peroxide radical, and nitrogen dioxide radical) and strong oxidants (e.g., peroxynitrite and hydrogen peroxide). These free radicals and oxidants have been shown to oxidatively modify proteins, nucleic acids and lipids to the point of causing cell injury and death.

Growing evidence supports the idea that over-expression or increased activity of mammalian heme peroxidases are involved in the pathogenesis and progression of a variety of diseases. For example, increased neutrophil-derived myeloperoxidase (MPO) activity has been found in atherosclerosis, Alzheimer's disease, Parkinson's disease, multiple sclerosis, IBD, kidney disease and rheumatoid arthritis. Another immune cell derived peroxidase, eosinophil peroxidase (EPO), also causes severe respiratory damage in asthma. The relationship between peroxidase and cardiovascular disease is so strong that a measured increase in heme peroxidase (MPO) has been used as a biomarker for the diagnosis and prognosis of cardiovascular disease. In spite of the recent growth in the evidence that heme peroxidases play important roles in the pathogenesis of vascular disease, effective therapies targeting aberrant heme peroxidase activity are largely lacking.

Mammalian heme peroxidases, including MPO, utilize hydrogen peroxide (H₂O₂) as a substrate to catalyze oxidation reactions with a variety of compounds through either a one-electron oxidation cycle or two-electron oxidation cycle. The mechanisms mediating peroxidase activity are as follows. In the inactive native state, the Fe ion in the active site of mammalian heme peroxidases is in the Fe⁺ state. This ferric ion reacts with H₂O₂ to form compound I, an oxy-ferryl-cation radical (PFe⁴⁺═O). Compound I reacts with halides (Cl⁻¹ and Br⁻¹) or pseudohalides (SCN⁻¹) via direct, two-electron reduction to form hypochlorous acid (HOCl), hypobromous acid (HOBr), and hypothiocyanite (HOSCN), respectively. As these potent oxidants leave the active site, the heme peroxidases are reduced back to Fe³⁺ and the cycle starts over again with the arrival of a second H₂O₂.

Compound I can also react with organic and inorganic substrates such as aromatic amino acids, derivatives of indole and a variety of other species (i.e., nitrite, ascorbate and urate) via two, sequential one-electron reductions. In this reaction sequence, nitrite or tyrosine reduces Compound Ito form compound II (Fe⁴⁺═O), which yields a nitrogen dioxide radical or a tyrosyl radical, respectively. Compound II can be further reduced by one electron back to the Fe³⁺ state by a second nitrite or tyrosine.

It is important to note that heme peroxidases can generate both oxidants and free radicals through direct oxidation of biological molecules. This makes them one of the most potent sources of oxidative stress. It has been shown that heme peroxidase-derived oxidants and free radicals oxidatively modify proteins to chlorotyrosine, bromotyrosine, nitrotyrosine, dityrosine, thiol oxidation products and haloamine, DNA molecules to 5-chlorouracil, and lipids to halohydrins, lysophospholipids, alpha-halo-fatty aldehydes, and other lipid peroxidation products.

As peroxidase-generated oxidants and radicals induce cell injury and death, inhibition of such chronic increases in aberrant peroxidase activity should, in turn, decrease chronic inflammation. Several research programs have worked on developing inhibitors for heme peroxidases over the last several decades. For the most part this research has focused on three lines of investigation. The first line of research focuses on hydrazine (RNHNH₂) and hydrazide (RCONHNH₂) derivatives that irreversibly inhibit heme peroxidase activity. However, these compounds are considered “suicide substrates” of peroxidase and inactivate the enzyme by irreversibly inhibiting the heme center.

The second line of research focuses on hydroxamic acids [RCNOHOH or RC(O)NHOH] and indole type compounds that reversibly inhibit peroxidase activity. Hydroxamic acids reduce Compound I and II. They also inhibit H₂O₂ binding to the heme peroxidase to inhibit formation of Compound I. Some of the indole derivatives (such as tryptophan, tryptamine and melatonin) also rapidly reduce compound I and inhibit the two-electron oxidation cycle of peroxidase. A third line of investigation focuses on another class of compounds that inhibit peroxidase via scavenging heme peroxidase oxidation products, for example, vitamin E and polyphenols scavenge nitrogen dioxide radicals.

Although MPO plays an important role in fighting infection, it is also believed to play a causal role in the development of atherosclerosis. Several clinical studies show that plasma MPO concentrations directly correlate with increased risk of arteriosclerosis, acute myocardial infarction and even heart failure. Immunofluorescent studies show that sickle cell disease (SCD) increases MPO deposition in the subendothelial spaces in the lungs of patients who have died from complications of SCD. The fact that MPO was observed to co-localize with 3-nitrotyrosine in these studies means that when MPO is released and trapped in the vessel wall, it remains fully capable of generating potent oxidants (i.e., .NO₂) to nitrate protein tyrosines.

Unfortunately, the progress made in understanding the mechanisms by which MPO impairs vascular function, has not translated into the development of effective therapies. Even though aggressive efforts have been taken, most of the agents or drugs that have been developed to date fall short for several reasons. Several strategies have been employed. For example, azide, hydrazides and hydroxamic acids have been used to irreversibly inhibit MPO by modifying the heme site. Indole derivatives have been used because they effectively compete with Cl⁻, SCN⁻ to prevent Compound I from generating HOCl and HOSCN. Phenolic compounds have been used because they effectively scavenge compound I and II.

However, all of these agents have side effects that limit their use to in vitro or cell culture studies. It is unclear if the intrinsic toxic nature of the compounds (such as hydrazine or hydrazide) or the toxicity of products generated after oxidation by peroxidase makes the compounds essentially worthless as therapeutic agents. In the instances where the agents have been used in animal models, they were observed to be either directly toxic or were converted into toxic compounds. For instance, heme poisons have been shown to inhibit mitochondrial respiration, obviously a deleterious effect on organism survivability. Even though indole derivatives are effective for scavenging Compound I, MPO oxidizes them to an indole radical that is both toxic and capable of increasing oxidative stress. Finally, even though phenolic agents effectively scavenge Compound I, MPO oxidizes them to phenoxyl radicals that are highly toxic and capable of increasing oxidative stress via oxidative modification of proteins and lipids. Such outcomes underscore the importance of developing specific MPO inhibitors that can be used not only for treating vascular disease but also investigating mechanisms by which MPO increases vascular disease.

In U.S. Pat. Nos. 8,673,847 and 8,937,039, each of which is incorporated by reference herein in its entirety, we disclosed a series of tripeptide inhibitors of MPO toxic oxidant production. These agents exhibit significant improvements over other MPO inhibitors. However, these agents were claimed to not target other pathways that may also contribute to inflammatory activity.

The advent of both systems biology and precision medicine has stimulated a rethink on the process of therapeutic drug design and polypharmacology. More recently, the definition of polypharmacology has morphed to represent therapeutic drugs that have been designed deliberately for multi-targeting that affords beneficial effects to the patient. This emerging effort has been labelled “Systems Pharmacology” and the products are referred to as multi-target or systems pharmacology drugs (see Pritchard et al., Systems Pharmacology; When multi-targeting is advantageous, Drug Discovery World, 2018).

Accordingly, there is a need in this field to obtain improved agents that can effectively prevent or reduce peroxidase-dependent oxidative stress in the in vivo setting, while further targeting other pathways related to the disease or inflammation process.

### BRIEF SUMMARY

We disclose herein a new class of drug designated as “systems chemico-pharmacology drugs” (SCPDs). SCPDs work by targeting a druggable biomolecular site (the first target), resulting in a significant change in the properties and function of the first target. In the process, the SCPD itself is chemically modified and activated. Subsequently, the resulting modified/activated SCPD interacts with one or more second targets, each of which is also modified in a manner that is beneficial for the patient.

Accordingly, the invention is directed to a systems chemico-pharmacology drug (SCPD) for treating a disease or condition characterized by an increased peroxidase activity in a subject, the SCPD configured such that:
i. the SCPD interacts with a first target that comprises a druggable biomolecular site;
ii. the SCPD modifies the properties and function of the first target and is itself chemically modified and activated, and
ii. the chemically modified/activated SCPD interacts with one or more second targets, thus modifying each of the second targets' functions; whereby the disease or condition is treated in the subject.

The druggable biomolecular site is a protein, a peptide, a DNA segment, an RNA segment, or a metabolite. A preferred druggable biomolecular site is an active site of an enzyme such as peroxidase. In certain embodiments, the peroxidase is a myeloperoxidase (MPO) or an eosinophil peroxidase (EPO).

The SCPD may be an agonist or an antagonist of the first target.

The SCPD may be chemically modified after administration to a subject by being oxidized, being reduced, forming a radical, forming a salt, or undergoing energetic excitation. In certain embodiments, the chemically modified SCPD comprises a stabilized free radical. The chemically modified version of the SCPD may be capable of auto-scavenging by forming a homodimer of chemically modified SCPDs via an oxidized linkage. Alternatively, the chemically modified SCPD is capable of scavenging by forming a heterodimer with the second target via an oxidized linkage.

Each of the second targets is preferably a peptide or protein such as a pro-inflammatory peptide or protein, and wherein the peptide or protein's function is modified to reduce its pro-inflammatory activity. Exemplary pro-inflammatory peptides and proteins include glutathione, HMGB1, RAGE, TRL4, NRf2 or KEAP-1.

In certain embodiments, the SCPD has the peptide-based formula AA(n),

wherein n is 2-5;

the peptide comprising:
i. an N-terminus amino acid that includes a basic side chain;
ii. an amino acid at any one of positions 2-5 that includes a polar, non-polar, aromatic ring or hetero-atom side-chain that can stabilize a radical species and is configured such that the amino acid at position 2-5 can participate in direct radical transfer from the heme porphyrin of the MPO's active site to the amino acid's side-chain, thus yielding the chemically modified SCPD;
iii. an amino acid at any one of positions 2-5 including a side chain that can interact with the MPO's active site through one or more of ionic, dipolar, pi-pi, hydrophobic or hydrophilic interactions facilitating radical transfer from the heme porphyrin of the MPO's active site to the SCPD; and
iv. an amino acid at any one of positions 2-5 that includes a side chain containing a heteroatom that stabilizes the radical on the chemically modified SCPD;
   wherein the chemically modified SCPD is configured to: auto-scavenge by forming a homo dimer via an oxidized linkage with another chemically modified SCPD; or, optionally, to scavenge by forming a hetero dimer via an oxidized linkage with another peptide or protein.

In certain embodiments, the SCPD can act as a myeloperoxidase (MPO) inhibitor.

Certain preferred SCPDs are peptides having the formula KLC, KVC, or KVVC.

According to the invention, the SCPD is a tripeptide KXZ having the formula AA₁-AA₂-AA₃, wherein:
AA₁ (K) is the N-terminus amino acid that includes a basic side chain;
AA₂ (X) is the amino acid that includes a polar, non-polar, aromatic ring or hetero-atom side chain that stabilizes the radical species and participates in direct radical transfer from the heme porphyrin of the MPO's active site, thus yielding the chemically modified SCPD, and
AA₃ (Z) is the amino acid possessing the heteroatom capable of stabilizing the free radical formed in said tripeptide.

The SCPD preferably includes a protecting group at one or both termini. In certain embodiments, the protecting group coupled to the SCPD's amino terminus is an N-acetyl group and the protecting group coupled to the SCPD's carboxyl terminus is an amide protecting group.

The N-terminus amino acid of preferred SCPDs is lysine.

Exemplary SCPDs are, accordingly, KLC, KVC or KVVC or modified variants Ac-KLC-amide, Ac-KVC-amide or Ac-KVVC-amide.

In certain embodiments, the SCPD is the retro or retro-inverso formula of the formula AA(n).

In certain embodiments the peptide can be a cyclic peptide.

In certain embodiments the SCPD is a deuterated (either partially or per-deuterated) compound/peptide.

Optionally, the formula AA(n) is comprised by L-amino acids or D-amino acids. The SCPD formula AA(n) may include natural or one or more artificial amino acids.

This can be appreciated, exemplary SCPDs comprise a tripeptide designated as KXZ. KXZ requires amino acid #1 (AA₁, K) to be a native amino acid or an artificial amino acid (aa) with a basic side-chain such as an amine functional group (e.g., Lysine (K)). Amino acid #2 (AA₂, X) is a native or artificial amino acid that may contain a polar, non-polar, or aromatic amino acid. Amino acid #3 (AA₃, Z) is a native or artificial amino acid that possesses a heteroatom that can stabilize a free radical that then can directly go on to react with a peptide or protein to modify the properties and function of such peptides and proteins. In a non-limiting example, the free radical forms a heteroatom-carbon bond in the form of a homo- or heterodimer of the tripeptide.

In another aspect, the disclosure encompasses a method of treating a disease or condition in a subject. The method includes the step of administering to the subject a systems chemico-pharmacology drug (SCPD), whereby the SCPD interacts with a first target that includes a druggable biomolecular site. The SCPD modifies the properties of the first target and is itself chemically modified/activated. The chemically modified/activated SCPD subsequently interacts with one or more second targets, thus modifying each of the second target's function. In this manner, the disease or condition is effectively treated in the subject.

In some embodiments, the druggable biomolecular site is a protein, a peptide, a DNA segment, an RNA segment, or a metabolite.

In some embodiments, the SCPD is an agonist or an antagonist of the first target.

In some embodiments, the SCPD is chemically modified by being oxidized, being reduced, forming a radical, forming a salt, or undergoing energetic excitation. A non-limiting example of undergoing energetic excitation is undergoing electron transfer excitation.

In some embodiments, the SCPD includes a tripeptide KXZ having the formula AA₁-AA₂-AA₃, wherein AA₁ (K) is an amino acid comprising a basic side chain, AA₂ (X) is a polar, non-polar or aromatic amino acid, and AA₃ (Z) is an amino acid possessing a heteroatom that is capable of stabilizing a free radical. In some such embodiments, the heteroatom stabilizes the free radical, which can directly go on to react with a peptide or protein to modify the properties and function of such peptides and proteins. In a non-limiting example, the free radical forms a heteroatom-carbon bond in the form of a homo- or heterodimer of the tripeptide.

In some embodiments, the tripeptide KXZ may further include a protecting group at one or both termini.

In some embodiments, SCPD consists of the tripeptide KXZ.

In some embodiments, the SCPD consists of the tripeptide KXZ and a protecting group coupled to its amino terminus, a protecting group coupled to its carboxyl terminus, or both. In some such embodiments, the SCPD includes an acetyl protecting group coupled to its amino terminus, an amide protecting group coupled to its carboxyl terminus, or both.

In some embodiments, AA₁ of the KXZ tripeptide is lysine. In some such embodiments, the KXZ tripeptide is lysyltyrosylcysteine (KYC) or N-acetyl lysyltyrosylcysteine amide (Ac-KYC-amide).

In some embodiments, the first target includes a peroxidase. In some such embodiments, the peroxidase is myeloperoxidase (MPO) or eosinophil peroxidase (EPO).

In some embodiments, the chemically modified SCPD includes the tripeptide KXZ*, where Z* is a radical located on the side-chain heteroatom of Z.

In some embodiments, one or more of the second targets can be a protein. In some such embodiments, the protein is a pro-inflammatory protein, and the second target's function is modified to reduce its pro-inflammatory activity. In some embodiments, the protein is HMGB1, RAGE, TRL4, NRf2 or KEAP-1.

In some embodiments, the SCPD includes the retro or retro-inverso formula of the tripeptide KXZ.

In some embodiments, the tripeptide KXZ comprises L-amino acids or D-amino acids.

In some embodiments, the tripeptide KXZ comprises natural or artificial amino acids.

In certain embodiments the peptide can be a cyclic peptide.

In certain embodiments the SCPD is a deuterated (either partially or per-deuterated) compound/peptide.

In some embodiments, the disease or condition treated is a disease or condition associated with aberrant peroxidase activity in the subject.

In some embodiments, the disease or condition treated is wound inflammation, hypersensitivity, digestive disease, cardiovascular disease, neuronal disease, lung disease, autoimmune disease, degenerative neurological disease, degenerative muscle disease, infectious disease, disease associated with graft transplantation, allergic disease, musculo-skeletal inflammation, sepsis, hypertension, peripheral vascular disease, pulmonary inflammation, asthma, atherosclerosis, diabetes, persistent pulmonary hypertension, sickle cell disease, neurodegenerative disease, multiple sclerosis, Alzheimer's disease, lung cancer, lupus, ischemic heart disease, Parkinson's disease, Crone's disease, inflammatory bowel disease, necrotizing enterocolitis, arthritis, polymyocytis, cardiomyopathy, psoriasis, amyotrophic lateral sclerosis, muscular dystrophy, cystic fibrosis, attention deficiency hyperactive disorder, acute lung injury, acute respiratory distress syndrome, swine flu, heart failure, chemotherapy-induced heart failure, arthritis, rheumatoid arthritis, acute myocardial infarction, traumatic brain injury (TBI), chronic traumatic encephalopathy (CTE), ischemic or hemorrhagic stroke, or bronchopulmonary dysplasia.

In another aspect, the disclosure encompasses the use of any of the compositions of claims described above to treat the disease a disease or condition associated with aberrant peroxidase activity in the subject.

In yet another aspect, the disclosure encompasses the use of any of the compositions described above to manufacture a medicament to treat a disease or condition associated with aberrant peroxidase activity in the subject.

In some embodiments directed to the uses of the compositional systems, the disease or condition treated is wound inflammation, hypersensitivity, digestive disease, cardiovascular disease, neuronal disease, lung disease, autoimmune disease, degenerative neurological disease, degenerative muscle disease, infectious disease, disease associated with graft transplantation, allergic disease, musculo-skeletal inflammation, sepsis, hypertension, peripheral vascular disease, pulmonary inflammation, asthma, atherosclerosis, diabetes, persistent pulmonary hypertension, sickle cell disease, neurodegenerative disease, multiple sclerosis, Alzheimer's disease, lung cancer, lupus, ischemic heart disease, Parkinson's disease, Crone's disease, inflammatory bowel disease, necrotizing enterocolitis, arthritis, polymyocytis, cardiomyopathy, psoriasis, amyotrophic lateral sclerosis, muscular dystrophy, cystic fibrosis, attention deficiency hyperactive disorder, acute lung injury, acute respiratory distress syndrome, swine flu, heart failure, chemotherapy-induced heart failure, arthritis, rheumatoid arthritis, acute myocardial infarction, traumatic brain injury (TBI), chronic traumatic encephalopathy (CTE), ischemic or hemorrhagic stroke, or bronchopulmonary dysplasia.

Further objects, features and advantages of the invention will be apparent from the following detailed description when taken in conjunction with the accompanying drawings.

### BRIEF DESCRIPTION OF THE DRAWINGS

The disclosure will be better understood and features, aspects and advantages other than those set forth above will become apparent when consideration is given to the following detailed description. Such detailed description makes reference to the following drawings.

FIG. 1. This cartoon illustrates potential cellular mechanisms by which KYC inhibits MPO toxic oxidant production. Hydrogen peroxide is a substrate and initially activates MPO. MPO oxidizes a variety of substrates (Cl⁻, NO₂⁻, tyrosine [Y]) to toxic oxidants. N-Acetyl-lysyltyrosylcysteine amide (KYC) is a tripeptide substrate that competes with endogenous substrates from the active site of MPO and in so doing is oxidized. However, after being oxidized, the KYC radical (.) auto-scavenges by forming a KYC homo-disulfide. Physiological concentrations of GSH reduce KYC homo-disulfide to a monomer that can once again be oxidized by MPO. Additionally, glutathione reductase (GR) can reduce KYC homo-dimers and hetero-dimers to monomers in the presence of GSH, suggesting that GR-dependent reduction is dependent on thiol exchange.

FIG. 2. Hyperoxia increases the infiltration of inflammatory myeloid cells in the lungs which increases myeloperoxidase (MPO), 3-chlorotyrosine (Cl-Tyr), and 3-nitrotyrosine (NO₂-Tyr). Rat pups were housed with nursing dams in either room air or >90% oxygen environment. (A) Lungs obtained at P10 showed increased MPO positive myeloid cell infiltration (5.9±2.9/HPF vs. 0.3±0.3/HPF, n=8) after hyperoxia-exposure. (B) The relative MPO expression levels increased at P10 under hyperoxia (4.2±3.9 vs. 1.0±0.4, n=15). (C) As a result of the hyperoxia-induced increases in MPO, Cl-Tyr levels increased by (2.4±1.0 vs. 1.0±0.2, n=9). (D) NO₂-Tyr levels also increased as the result of the hyperoxia induced increases in MPO. Red arrows: MPO-(+) cells; HOX: hyperoxia (O2>90%); NOX: normoxia (room air); *=p<0.05.

FIG. 3. Hyperoxia impairs the growth of neonatal lungs resulting in simplification of lung structure at P10. (A) Hyperoxia decreased radial alveolar counts (RAC) significantly (4.6±0.5 vs. 6.3±0.6, n=12). (B) Hyperoxia decreased the number of secondary septation (7.3±2.8/HPF vs. 15.1±1.8, n=9). (C) Hyperoxia decreased capillary density (5.9±3.1/HPF vs. 11.8±1.8/HPF, n=11). (D) Hyperoxia increased the mean linear intercept (82.2±12.0 μm vs. 60.7±10.8 μm, n=11). (E) Hyperoxia decreased CD31 levels in lung lysates (1.0±0.1 vs. 0.5±0.3, n=9). HOX: hyperoxia (O₂>90%); NOX: normoxia (room air); *=p<0.05.

FIG. 4. KYC decreases myeloperoxidase (MPO) levels and MPO activity in neonatal lungs exposed to hyperoxia. (A) MPO-positive myeloid cell count decreased (2.0±0.7/HPF vs 0.8±0.7/HPF, n=9). (B) MPO levels decreased (1.0±0.3 vs 0.6±0.1, n=9). (C) Cl-Tyr levels decreased (1.0±0.0 vs 0.7±0.1, n=9), (D) NO₂-Tyr levels decreased (1.0±0.1 vs 0.6±0.2, n=9). HOX: hyperoxia (O₂>90%); *=p<0.05.

FIG. 5. KYC attenuates hyperoxia-induced alveolar simplification in neonatal lungs. (A) KYC treatment increased RAC from 4.2±0.6 (n=9) for the PBS-treated HOX group to 6.3±0.4 (n=12) for the KYC-treated HOX group. (B) KYC treatment increased secondary septation from 5.0±1.3/HPF (n=9) for the PBS-treated HOX group to 7.8±1.2/HPF (n=12) for the KYC-treated HOX group. (C) KYC treatment increased capillary count from 5.2±1.0/HPF (n=9) for the PBS-treated HOX group to 9.7±3.0 (n=12) for the KYC-treated HOX group. (D) KYC treatment decreased MLI from 95.9±9.1 μm (n=9) for the PBS-treated HOX group to 74.1±4.7 μm (n=12) for the KYC-treated HOX group. (E) KYC treatment increased CD31 levels from 1.0±0.1 (n=9) for the PBS-treated HOX group to 1.2±0.3 for the KYC-treated HOX group. HOX: hyperoxia (O₂>90%, n=9). *=p<0.05.

FIG. 6. Hyperoxia increases HMGB1 levels in neonatal pup lungs while KYC treatment decreases HMGB1 levels in the lungs isolated from hyperoxic neonatal rat pups. (A) Hyperoxia increases HMGB1 levels in neonatal pup lungs (1.0±0.2 vs 2.7±1.7, n=12), (B) KYC decreases HMGB1 levels in the lungs isolated from hyperoxic neonatal pups (1.0±0.2 vs 0.6±0.2, n=11, *=p<0.05).

FIG. 7. Hyperoxia increases RAGE and TLR4 expression in the lungs of neonatal rat pups while KYC treatments decrease RAGE and TLR4 expression in the lungs of hyperoxic neonatal rat pups. (A) Hyperoxia increases the expression of RAGE neonatal rat pup lungs (1.0±0.3 vs 2.5±0.7, n=12). (B) KYC treatment of hyperoxic neonatal rat pups decreased the expression of RAGE in lungs isolated from hyperoxic neonatal rat pups (1.0±0.2 vs 0.5±0.2, n=16). (C) Hyperoxia increases TLR4 expression in the lungs of hyperoxic neonatal rat pups (1.0±0.2 vs 2.6±0.6, n=10). (D) KYC treatment decreased TLR4 expression in the lungs of hyperoxic neonatal rat pups (1.0±0.1 vs 0.8±0.2, n=12). *=p<0.05.

FIG. 8. KYC Increases Survival of Neonatal Pups Raised in Hyperoxia. Kaplan-Meier survival curves for neonatal rat pups exposed to chronic hypoxia P1-P10 as indicated in Methods. Data are plotted from 29 pups per PBS treated hyperoxic neonatal rats (red curve) and 37 pups per KYC treated hyperoxic neonatal rats (blue curve). These data show that KYC treatment increases survivability of the hypoxic neonatal rat pups from 82.8% for the PBS group to 97.3% for KYC group at P10 (***=p<0.001).

FIG. 9. GSH Reductase (GR) reduces KYC-KYC homo-disulfide and KYC-GSH hetero-disulfide to KYC monomer. Trace A=HPLC separation of KYC, KYC-GSH hetero-disulfide and KYC-KYC homo-disulfide. Trace B=separation of peptide mixture from A after incubation with GR (3.8 μM) and NADPH (1 mM). Disappearance of KYC-GSH and KYC-KYC hetero- and homo-disulfides in Trace B demonstrates that both disulfides are reduced by GR/NADPH reaction mixtures in the presence of GSH. KYC-KYC homo-disulfides were not reduced to KYC monomers by GR/NADPH reaction mixtures or by either reagent alone (data not shown). These data suggest KYC can exploit enzymes in the GSH pathway for reduction of inactive homo- and hetero-disulfides to active KYC monomers when GSH is present.

FIG. 10. Bronchopulmonary Dysplasia is Caused by a Destructive Cycle that Contains Five Components: Excess Oxygen, Myeloid Cells, MPO, HOCl and HMGB1. Red Cycle: Myeloid cells are recruited from the circulation into the inflamed lung. Here they become activated and release MPO and HMGB1. HMGB1 binds to and activates RAGE and TLR4, which induce even greater levels endothelial cell oxidative stress and inflammation. Activated myeloid cells generate H₂O₂, which serves as a substrate and initially activates MPO allowing it to generate HOCl. HOCl is a potent oxidant, increases pulmonary endothelial cell injury and oxidative damage that together increases necrosis. Necrotic cells passively release HMGB1 which initiates a second wave of myeloid cell recruitment and vascular oxidative damage and inflammation. Blue Cycle: KYC, an MPO substrate, effectively competes with endogenous substrates to inhibit MPO toxic oxidant (HOCl) production. MPO oxidation of KYC generates a KYC radical (□) that auto-scavenges by forming a KYC homo-disulfide. In the presence of GSH and NADPH, GR reduces KYC homo-disulfides to KYC monomers that can be once again oxidized by MPO to inhibit HOCl production.

FIG. 11. KYC IC50 inhibition of MPO HOCl production at varied pH.

FIG. 12. KWC IC50 inhibition of MPO HOCl production at varied pH.

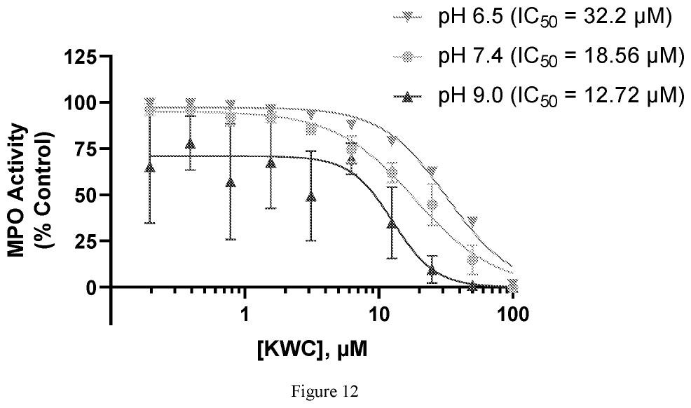

FIG. 13. KFC IC50 inhibition of MPO HOCl production at varied pH.

FIG. 14. KLC IC50 inhibition of MPO HOCl production at varied pH.

FIG. 15. KVC IC50 inhibition of MPO HOCl production at varied pH.

FIG. 16. KVVC IC50 inhibition of MPO HOCl production at varied pH.

FIG. 17. MPO-dependent KYC Thiolation of HMGB1. HMGB1 was incubated with biotin-KYC, H2O2, and MPO as indicated in the key above and then separated by non-reducing SDS-PAGE. 1A) MPO thiolation of HMGB1 was detected with a fluorescent-labeled streptavidin. Sample split and one aliquot treated with DTT to reduce KYC disulfide to a KYC monomer and remove it from HMGB1. 1B) HMGB1 was incubated with Biotin-KYC, H2O2, and MPO as indicated in the key above and separated by non-reducing SDS-PAGE.

FIG. 18. KYC facilitates HMGB1 thiolation in endothelial cells in the presence of MPO and H₂O₂. Cell lysates were immunoprecipitated with HMGB1 antibody then blotted with streptavidin-HRP.

FIG. 19. Thiolation of HMGB1 in endothelial cell needs the existence of both MPO and H₂O₂. The HMGB1 thiolation occurs mainly within the cell with small amounts also released into the medium. HMGB1 thiolation is identified by streptavidin-HRP.

FIG. 20. KYC treatment reduces cell death in hyperoxia-exposed rat pup lungs. The increased acetylated HMGB1 in KYC treated rat lung indicates the HMGB1 is in secreted form from endothelial cells.

FIG. 21. Thiolation of HMGB1 by KYC in endothelial cells needs the existence of both MPO and H₂O₂. KYC directly thiolates the protein Keap1. Thiolation is identified by streptavidin-HRP.

FIG. 22. KYC treatment decreases oxidative stress from hyperoxia with decreased Nrf2 levels in the hyperoxia exposed rat pup lungs.

FIG. 23. KYC Decreased BPD in Hyperoxic Pups. (A) Representative immunohistochemistry images of lung sections from PBS- (Left panel) and KYC-treated, hyperoxic neonatal rat pups. Images show that KYC decreased MPO+ myeloid cell counts relative to MPO+ cell counts in lungs of hyperoxic pups (n=9). The bar chart shows the mean±SD of MPO+ cell counts in lung sections from hyperoxic pups treated with PBS and KYC, respectively. (B) Immunoblots show that lung lysates from KYC-treated hyperoxic pups contain less MPO protein than lung lysates from PBS-treated hyperoxic pups (n=9). The bar chart shows the mean±SD of the band densities for MPO relative to actin in lung lysates from hyperoxic pups treated with PBS and KYC. (C) Immunoblots show that lung lysates from KYC-treated hyperoxic pups have less Cl-Tyr immunoreactive proteins than lung lysates from PBS-treated hyperoxic pups (n=9). Quantification of immunoreactive Cl-Try protein densities was by scanning the entire lane and normalizing the integrated scanned profile to actin, an internal control (Hyperoxia=O₂>90%; *=p<0.05).

FIG. 24. KYC Prevented Alveolar Simplification in Lungs of Hyperoxic Pups. (A) Representative H&E images of lung sections showing alveolar and vascular simplification resulting from chronic hyperoxia that were reversed with KYC treatment. RAC are increased in lung sections from KYC-treated hyperoxic pups (left, n=9) compared to RAC levels in lung sections from PBS-treated hyperoxic pups (right, n=12). The bar chart shows the mean±SD for RAC from lung sections from hyperoxic pups treated with PBS and KYC. (B) Representative H&E images of lung sections from hyperoxic pups treated with PBS (left) vs. KYC (right). Arrows indicate lungs structures counted as secondary septa. Bar charts summarize secondary septation data and show that KYC treatment of hyperoxic pups increased secondary septation (right, n=9) compared to secondary septation in PBS-treated hyperoxic pups (left, n=12). The bar chart shows the mean±SD for secondary septa in lung sections from hyperoxic pups treated with PBS and KYC. (C) Representative H&E images showing capillary structures in lung sections from hyperoxic pups treated with PBS (left) vs. KYC (right). Arrows indicate lung structures counted as capillaries. The bar chart shows capillary counts are increased in KYC-treated hyperoxic pups (right, n=9) compared with counts in PBS-treated hyperoxic pups (left, n=12). (D) Representative H&E images of lung sections from PBS-treated (left) and KYC-treated (KYC) hyperoxic pups. The bar chart shows MLI are decreased in lung sections in KYC-treated hyperoxic pups (right, n=9) compared to MLI in lungs of PBS-treated hyperoxic neonatal rat pups (left, n=12). (E) Representative immunoblots for CD31 relative to actin, an internal loading control from lung lysates prepared from KYC-treated hyperoxic pups (right, n=9) and PBS-treated hyperoxic pups. (left, n−9). The bar chart shows the mean±SD of relative differences in CD31 band densities normalized to actin in lung lysates from PBS and KYC treated hyperoxic pups. (Hyperoxia=O₂>90%, *=p<0.05).

FIG. 25. KYC Decreased Oxidative DNA Damage in Lungs of Hyperoxic Pups. Representative images of lung sections stained for nuclei (top, DAPI, Blue), immunostained for 8-OH-dG (middle, Red) and combined (bottom) in hyperoxic pups treated with PBS (left) and KYC (right). The bar chart shows the mean±SD of the relative changes in fluorescent densities for 8-OH-dG in lung sections from hyperoxic pups treated with PBS and KYC (PBS, n=7; KYC, n=8, *=p<0.05).

FIG. 26. KYC Decreased Cyclooxygenase-1 (Cox1) and -2 (Cox2) Expression in Lungs of Hyperoxic Pups. Representative immunoblots showing that KYC decreased hyperoxia-induced increases in Cox1 (n=6) and Cox2 (n=6) in lungs of hyperoxic pups. The bar chart shows relative changes in mean±SD of Cox1 and Cox2 band densities relative to β-Actin showing that KYC treatment reduced Cox1 and Cox2 expression in hyperoxic pups. (▪: Hyperoxia+PBS; ♦: Hyperoxia+KYC; *=p<0.05).

FIG. 27. KYC Decreased HMGB1 Release in Lungs of Hyperoxic Pups. Representative immunoblot for HMGB1 and Actin in lung lysates from hyperoxic pups treated with PBS (left) or KYC (right). These immunoblots show that KYC treatment decreased HMGB1 release in lung lysates isolated from hyperoxic pups relative to differences in Actin, an internal loading control. The bar chart shows relative changes in the mean±SD of HMGB1 band densities relative to the band densities of Actin in lung lysates from hyperoxic pups treated with PBS or KYC (▪: Hyperoxia+PBS, n=10; ♦: Hyperoxia+KYC; n=11, *=p<0.05).

FIG. 28. KYC Decreased RAGE and TLR4 in Lungs of Hyperoxic Pups. (A) Representative immunoblots for RAGE in lung lysates from hyperoxic pups treated with PBS and KYC (n=16, *=p<0.05). The bar chart presents the mean±SD of RAGE band densities normalized to actin and show that KYC decreased RAGE expression in lung lysates of hyperoxic pups. (B) Representative immunoblots for TLR4 expression in lungs lysates from hyperoxic pups treated with PBS and KYC (n=10). The bar chart presents the mean±SD of TLR4 band densities normalized to actin and show that KYC treatment decreased TLR4 expression in lung lysates of hyperoxic pups. (n=12, *=p<0.05).

FIG. 29. Effects of Oxidative Stress on KYC Thiylation of RLMVEC Proteins. (A) Streptavidin affinity blot of KYC thiylated proteins from cell lysates prepared from RLMVEC cultures treated with B-KYC (baseline), B-KYC+MPO+H₂O₂ (MPO-dependent), and B-KYC+H₂O₂ (H₂O₂-dependent). (B) Immunoblot for Nrf2 in RLMVEC cultures treated as described in A. (C) Immunoblot for Keap1 in RLMVEC cultures treated as described in A. (D) Immunoblot for HMGB1 in RLMVEC cultures treated as described in A.(E) Immunoblot for β-Actin in RLMVEC cultures treated as described in A.(F) Bar chart showing relative levels of KYC thiylation, Nrf2, Keap1, and HMGB1 as a function of β-Actin (n=2, *=p<0.05, statistical analysis was preformed by ANOVA with appropriate post hoc tests). These data show that MPO-dependent oxidation of KYC increases KYC thiylation in RLMVEC proteins cells that are proximal to MPO, while H₂O₂-dependent oxidation induces a slight if any increase in KYC thiylation of RLMVEC proteins.

FIG. 30. MPO Oxidizes KYC to a KYC Thiyl Radical That Thiylates HMGB1. (A) ESR spectrum of KYC thiyl S°-DMPO generated by an MPO reaction mixture containing MPO (120 nM), H₂O₂ (50 μM) and KYC (30 μM). (B) Streptavidin-affinity blot of KYC thiylated HMGB1 and MPO in a split sample, one half treated neat and other half treated with DTT (100 mM) to reduce disulfide bonds. The affinity blot shows DTT reduced B-KYC-thiylated HMGB1 and B-KYC-thiylated MPO fluorescent band densities, confirming that the bond between KYC and HMGB1 and MPO were both disulfides.

FIG. 31. MPO Oxidation of KYC Increases KYC Thiylation of Extracellular HMGB1. (A) Streptavidin affinity blot of conditioned media from RLMVEC cultures treated with media, media+B-KYC, and media+MPO reaction system (MPO=120 nM; H₂O₂=50 μM)+B-KYC. The affinity blot shows that the MPO reaction system+B-KYC predominately thiylates HMGB1 released into the conditioned media relative to the levels of HMGB1 protein released (immunoblot for HMGB1). (B) Streptavidin affinity blot of cell lysates of RLMVEC cultures treated with media, media+B-KYC, and media+MPO reaction system (MPO=120 nM; H₂O₂=50 μM)+B-KYC. This affinity blot shows that the MPO reaction system+B-KYC thiylates low levels of HMGB1 in cell lysates. This conclusion is based on the relative levels of KYC thiylated HMGB1 in panel A vs. KYC thiylated HMGB1 as a function of HMGB1 protein in conditioned media vs. RLMVEC cell lysates.

FIG. 32. Effects of KYC Treatment on HMGB1 Association with TLR4 and RAGE in Lung Lysates from Hyperoxic Pups. (A) Immunoblots of TLR4 associated with HMGB1 in lung lysates from PBS- and KYC-treated hyperoxic pups. A non-depleting concentration of anti-HMGB1 antibody was used to pull-down HMGB1 from lung lysates. The pull-down was immunoblotted for TLR4 and HMGB1. These immunoblots show that the levels of TLr4 association with HMGB1 decreased in lung lysates from KYC-treated hyperoxic pups relative to the levels of TLR4 associated with HMGB1 in PBS-treated hyperoxic pups. The lower immunoblot shows that non-depleting levels of anti-HMGB1 antibody pull-down essentially equal levels of HMGB1 from each sample irrespective of how treatments modulated lung TLR4 expression (FIG. 28, supplemental data FIG. 42). (B) Immunoblots of RAGE associated with HMGB1 in lung lysates from PBS- and KYC-treated hyperoxic pups. A non-depleting concentration of anti-HMGB1 antibody was used to pull-down HMGB1 from lung lysates. The pull-down was immunoblotted for RAGE and HMGB1. These immunoblots show that the levels of RAGE association with HMGB1 decreased in lung lysates from KYC-treated hyperoxic pups relative to the levels of RAGE associated with HMGB1 in PBS-treated hyperoxic pups. The lower immunoblot shows that non-depleting levels of anti-HMGB1 antibody pull-down essentially equal levels of HMGB1 from each sample irrespective of how treatments modulate lung RAGE expression (FIG. 28, supplemental data FIG. 42). (C) The bar charts show the mean±SD of relative levels of TLR4 and RAGE associated with HMGB1 in lung lysates from PBS- and KYC-treated hyperoxic pups (n=4, *=p<0.05).

FIG. 33. Effects of KYC on HMGB1 Thiol Oxidation State and Lysyl-Acetylation. (A) Representative immunoblots for cysteine sulfonyl on HMGB1 in lung lysates from normoxic and hyperoxic pups. Bar chart presents mean±SD of relative levels of cysteine sulfonyl on HMGB1 in lung lysates from normoxic and hyperoxic pups. These data show that hyperoxia decreased the levels of cysteine sulfonyl on HMGB1 in lung lysates. (B). Representative immunoblots for cysteine sulfonyl on HMGB1 in lung lysates from hyperoxic pups treated with PBS or KYC. Bar chart presents mean±SD of relative levels of cysteine sulfonyl on HMGB1 in lung lysates from hyperoxic pups treated with PBS or KYC. These data show that KYC treatment increased the levels of cysteine sulfonyl on HMGB1 in lung lysates of hyperoxic pups. (C) Activated immune cells are the predominant source of lysyl-acetylated HMGB1. Dead and dying cells are the predominant source of non-acetylated HMGB1. Representative immunoblots for lysyl-acetylated residues on HMGB1 immunoprecipitated from lung lysates from hyperoxic pups. HMGB1 was immunoprecipitated with non-depleting concentrations of anti-HMGB1 antibody. The immunoblots show that KYC treatment increases lysyl-acetylated HMGB1 isoforms in lung lysates from hyperoxic pups. Where the dominate HMGB1 isoform in hyperoxic pups treated with PBS is non-acetylated HMGB1, which is released by dead and dying cells, the dominant HMGB1 isoform in lung lysates from KYC treated hyperoxic pups is lysyl-acetylated HMGB1. These data demonstrate that HMGB1 release in lungs of hyperoxic pups is shifted from dead and dying lung cells in PBS-treated hyperoxic pups to activated immune cells in KYC-treated hyperoxic pups.

FIG. 34. Effects of KYC Treatment on KYC Thiylation of Keap1, Keap1 S-Glutathionylation and Nrf2 Activation in Lungs of Hyperoxic Pups. (A) The streptavidin affinity blot for B-KYC-thiylated Keap1 and the immunoblot for Keap1 show that KYC treatment increased B-KYC-thiylation of Keap1 in lung lysates from hyperoxic pups. The bar chart shows the mean±SD of the relative levels of B-KYC-thiylated Keap-1 as a function to Keap1 in lung lysates of hyperoxic pups treated with PBS and KYC. These data show that KYC treatment increased KYC thiylation of Keap1 in the lungs of hyperoxic pups. (B) Immunoblot for S-glutathionylated Keap1 shows that KYC treatment increased Keap1 S-glutathionylation in lung lysates from hyperoxic pups. The bar chart shows the mean±SD of the relative levels of GS-thiylated Keap-1 as a function to Keap1 in lung lysates of hyperoxic pups treated with PBS and KYC. These data show that KYC treatment increased S-glutathionylated Keap1 (GS-Keap1) levels in lungs of hyperoxic pups. (C) The immunoblot shows the relative levels of Nrf2 in lung lysates from normoxic pups treated with PBS and KYC. The bar chart shows the mean±SD of the relative levels of Nrf2 as a function to actin in lung lysates of normoxic pups treated with PBS and KYC. These data show that KYC treatment increased Nrf2 activation in lungs of normoxic pups. (D) Representative immunoblots of relative levels of Nrf2 activation in lung lysates from hyperoxic pups treated with PBS and KYC. The bar chart shows the mean±SD of the relative levels of Nrf2 as a function to actin in lung lysates of hyperoxic pups treated with PBS and KYC. These data show that KYC treatment increased Nrf2 activation in lungs of hyperoxic pups.

FIG. 35. Effects of KYC on Antioxidant Enzyme Expression in Lungs from Hyperoxic Pups. Representative immunoblots show that KYC treatment increased HO-1, GST and Trx expression in lungs of hyperoxic pups. The bar charts show mean±SD of the relative levels of HO-1, GST and Trx as a function of actin in lung lysates of hyperoxic pups treated with PBS and KYC (n=9, *=p<0.05).

FIG. 36. Effects of KYC on Weight Gain and Survival. (A) Line plot showing means of weight gain of normoxic pups treated with KYC. Statistical analysis reveals that KYC increased weight gain in normoxic pups from P3 to P10 and that corrected multiple t-test analysis shows significant differences between means for different days in addition to significant differences between curves. (21% O₂, n=13, 21% O₂+KYC, n=14, *=p<0.05, **=p<0.01). (B) Survival analysis for normoxic pups and normoxic pups treated with KYC finds no significant differences in survival. (C) Line plot showing means of weight gain of hyperoxic pups treated with PBS or KYC. Statistical analysis reveals that KYC increased weight gain in normoxic pups from P3 to P10 and that corrected multiple t-test analysis shows a significant difference between means only for P10 in addition to a significant difference between curves. (>90% O₂, n=22, >90% O₂+KYC, n=27, *=p<0.05, **=p<0.01). (D) Survival analysis for hyperoxic pups treated with PBS or KYC finds significant differences in survival. These data show that KYC significantly improves survival of hyperoxic pups. (>90% O₂, n=22, >90% O₂+KYC, n=27, *=p<0.05).

FIG. 37. Hyperoxia increased inflammatory myeloid cell infiltration into the lungs, which increased myeloperoxidase (MPO), and 3-chlorotyrosine (Cl-Tyr). Rat pups were housed with nursing dams in either room air or >90% oxygen environment. (A) Lungs obtained at P10 from hyperoxic neonatal rat pups had more MPO+ myeloid cells (n=8) lungs from normoxic neonatal rat pups. (B) MPO expression was increased in lungs from hyperoxic neonatal rat pups at P10 compared to the levels of expression in normoxic neonatal rat pups (n=15). (C) As a result of the hyperoxia-induced increases in MPO, Cl-Tyr levels (n=9) increased in the lungs of hyperoxic neonatal rat pups. Red arrows: MPO+ myeloid cells; hyperoxia (O₂>90%); normoxia (room air); *=p<0.05.

FIG. 38. Hyperoxia impairs the growth of neonatal lungs resulting in the simplification of lung structure at P10. (A) Hyperoxia decreased radial alveolar counts (RAC) compare to the counts in the lungs of normoxic neonatal rat pups (n=12). (B) Hyperoxia decreased the number of secondary septation compare to the number in lungs of normoxic neonatal rat pups (n=9). (C) Hyperoxia decreased capillary density compare to the density in the lungs of normoxic neonatal rat pups (n=11). (D) Hyperoxia increased the mean linear intercept (MLI) compare to the MLI in the lungs of normoxic neonatal rat pups (n=11). (E) Hyperoxia decreased CD31 levels in lung lysates compare to the CD31 levels in the lungs of normoxic neonatal rat pups (n=9). HOX: hyperoxia (O₂>90%); NOX: normoxia (room air); *=p<0.05.

FIG. 39. Hyperoxia increased oxidative DNA damage in the lungs of neonatal rat pups. Chronic hyperoxia increased oxidative DNA damage in PBS-treated neonatal rat lungs based on increased immunofluorescent staining for 8-OH-dG (n=4). Red: 8-OH-dG; Blue: DAPI; *p<0.05.

FIG. 40. Hyperoxia increased Cyclooxygenase-1 (Cox1) and -2 (Cox2) Expression. Chronic hyperoxia increased both Cox1 (n=6) and Cox2 (n=6), an index of non-specific inflammation. Normoxia; ▪: Hyperoxia; *=p<0.05).

FIG. 41. Hyperoxia increased the release of HMGB1 in the lungs of neonatal rat pups. Hyperoxia increased the release of HMGB1 in lung lysates from neonatal pup lungs compared to the release of HMGB1 lung lysates from normoxic neonatal rat pups (n=12, *=p<0.05).

FIG. 42. Hyperoxia increased the expression of RAGE and TLR4 in the lungs of neonatal rat pups. (A) Hyperoxia increased the expression of RAGE compared to the levels of RAGE in the lungs of normoxic neonatal rat pups (n=12, *=p<0.05). (B) Hyperoxia increased the expression of TLR4 compared to the levels of TLR4 in the lungs of normoxic neonatal rat pups (n=12, *=p<0.05).

FIG. 43. Hyperoxia Decreased Nrf2 activation in the lungs of neonatal rat pups (n=15, *=p<0.05).

FIG. 44. Effects of GSH Reductase on KYC Homodisulfides and Heterodisulfides. GSH Reductase (GR) reduces KYC-KYC homodisulfide and KYC-GSH heterodisulfide to KYC monomer. Trace A=HPLC separation of KYC, KYC-GSH heterodisulfide, and KYC-KYC homodisulfide. Trace B=separation of peptide mixture from A after incubation with GR (3.8 μM) and NADPH (1 mM). The disappearance of KYC-GSH and KYC-KYC heterodisulfides and homodisulfides in Trace B demonstrates that GR/NADPH reaction mixtures can reduce both disulfides in the presence of GSH but not when GSH is absent (data not shown). These data suggest KYC exploits enzymes in the GSH pathway for the regeneration of active KYC monomers from inactive homodisulfides and heterodisulfides in the presence of GSH.

While the invention is susceptible to various modifications and alternative forms, specific embodiments have been shown by way of example in the drawings and are herein described in detail. The description of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

### I. IN GENERAL

This invention is not limited to the particular methodology, protocols, materials, and reagents described, as these may vary. It is also to be understood that the terminology used in this disclosure is for the purpose of describing particular embodiments only and is not intended to limit the scope of the present invention, which will be limited only by the language of the appended claims.

As used in this disclosure and in the appended claims, the singular forms “a”, “an”, and “the” include plural reference unless the context clearly dictates otherwise. The terms “a” (or “an”), “one or more” and “at least one” can be used interchangeably. The terms “comprising”, “including”, and “having” can also be used interchangeably.

Unless otherwise defined, all technical and scientific terms used herein have the same meaning as commonly understood by one of ordinary skill in the art to which this invention belongs. Chemical compound names that are commonly used and recognized in the art are used interchangeably with the equivalent IUPAC name. All publications and patents specifically mentioned in this disclosure are incorporated by reference for all purposes.

Although suitable methods and materials for the practice or testing of the present invention are described below, other methods and materials similar or equivalent to those described herein, which are well known in the art, can also be used and all cited references are incorporated herein by reference for all purposes.

As used herein, the term “amino acid” residue or sequence refers to abbreviations used herein for designating the amino acids based on recommendations of the IUPAC-IUB Commission on Biochemical Nomenclature (see Biochemistry (1972) 11:1726-1732). Also included are the (D) and (L) stereoisomers of such amino acids when the structure of the amino acid admits of stereoisomeric forms. The term “amino acid” encompasses the 20 naturally-occurring amino acids and, as well, the “unnatural amino acids,” which include any amino acid, modified amino acid, and/or amino acid analog that is not one of the 20 common naturally occurring amino acids.

As used herein, the term “peptide” refers to a polymer of amino acid residues. The terms apply to amino acid polymers in which one or more amino acid residues is an artificial chemical analogue of a corresponding naturally occurring amino acid, as well as to naturally occurring amino acid polymers. For example, “peptide” specifically includes the non-genetically-coded amino acids that either occur naturally or are chemically synthesized including, but not limited to synthetic .alpha.- and .beta.-amino acids known to one of skill in the art.

As used herein, the phrase “protecting group” refers to a chemical group that, when attached to a functional group in an amino acid (e.g. a side chain, an alpha amino group, an alpha carboxyl group, etc.) blocks or masks the properties of that functional group. Preferred amino-terminal protecting groups include, but are not limited to acetyl, or amino groups. Other amino-terminal protecting groups include, but are not limited to alkyl chains as in fatty acids, propenol, formyl and others. Preferred carboxyl terminal protecting groups include, but are not limited to groups that form amides or esters.

As used herein, the term “retro” as applied to an amino acid sequence refers to an amino acid sequence that is in reverse order of the original reference sequence. The term “inverso” as applied to an amino acid sequence refers to an amino acid sequence composed of D-amino acids as opposed to the parent L-sequence. Because the orientation of the side-chains in a retro-inverso analog is very similar to that in a reference L-sequence, there is a high probability of functional similarity between the two sequences.

As used herein, the term “subject” includes non-human mammals and humans.

As used herein, the phrase “therapeutically effective amount” means the amount of a compound that, when administered to a subject for treating a disease or disorder, is sufficient to affect such treatment for the disease or disorder. The “therapeutically effective amount” can vary depending on the compound, the disease or disorder and its severity, and the age, weight, etc., of the subject to be treated.

As used herein, the term “treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same.

### II. THE INVENTION

This disclosure is based on our discovery that certain peptide-based inhibitors of MPO are modified in the process of MPO inhibition such that they can subsequently target other pathways related to disease and/or inflammation. We have designated such agents as “systems chemico-pharmacology drugs” (SCPDs). We have demonstrated this phenomenon using a KXZ tripeptide (in KXZ amino acid #1 (AA₁, K) is a native amino acid or an artificial amino acid (aa) with a basic side-chain such as an amine functional group (e.g., Lysine (K)); Amino acid #2 (AA₂, X) is a native or artificial amino acid that may contain a polar, non-polar, or aromatic amino acid; and Amino acid #3 (AA₃, Z) is a native or artificial amino acid that possesses a heteroatom that can stabilize a free radical. The free radical itself may act as an activated species, and is stable enough to react with proximal proteins, peptides, metabolites or small molecules to effect further change. The free radical is not limited to a single target, but may effect change multiple independent targets. In a non-limiting example, the free radical goes on to form a heteroatom-carbon bond in the form of a homo- or heterodimer of the tripeptide) in a model of bronchopulmonary dysplasia. However, SCPDs can be used to treat a wide variety of diseases and conditions.

### Unique Properties of an SCPD

Both “systems pharmacology” and “polypharmacology” of therapeutic drugs are terms used as descriptors to describe efforts to overcome the one drug-one target model. Polypharmacology refers to a one drug-multi-target concept as utilized in drug repurposing. However, systems pharmacology refers to a broader concept best captured as one drug-multi targets associated with pathways and networks. Thus systems pharmacology is based on the rational design of drug therapies using information based on molecular, cellular and physiological complexity. This type of approach produces systems pharmacology drugs rooted in molecular interactions between a single drug and multi-targets present in defined pathways/networks.

A systems chemico-pharmacology drug (SCPD) has unique properties and must do the following: i) SCPD must target a druggable biomolecular site. This can be a protein/peptide, DNA, RNA, or metabolite or other small molecule. ii). The SCPD must modify the properties of the target, it can be an agonist or antagonist, that results in the target properties changing. iii) As a function of the SCPD-target interaction and change in target properties, the SCPD is itself chemically modified. iv) The modification can be oxidation/reduction, formation of a radical, salt formation, or any form of energetic excitation such as electron transfer excitation, to create a modified SCPD structure. v) this modified entity must then interact with specific new target(s) and modify the new target's function in a efficacious manner for the patient.

### Systems Chemico-Pharmacology Drugs Having the Generic Formula AA₍ₙ₎

In a first aspect, the invention is directed to small end-capped peptides with a generic formula of AA₍ₙ₎ (where AA can represent different amino acids, and n can be 2-5) that inhibit MPO. In addition we provide a set of physicochemical properties of individual amino acid that collectively contribute to inhibition of MPO. These same criteria (for the amino acids and thus collectively for the peptide) are also applicable to any organic molecule that will inhibit MPO toxic oxidant production. The peptide studies described herein were designed to better describe and understand the optimal sequence and physicochemical properties of individual amino acids used as constituents of MPO small peptide inhibitors. These same peptides also serve as inhibitors of other members of the peroxidase enzyme family such as eosinophil peroxidase.

Small peptides of generic formula AA(n) (where AA represents different amino acids, and n can be 2-5) are both substrates and inhibitors of MPO (alternatively, inhibitors of EPO). These peptides are reduced (gain an electron in the form of a radical) at the active site of MPO, which subsequently decreases the production of toxic oxidants that includes but not restricted to hypochlorous acid. All MPO activities (i.e., hypochlorous acid production) described herein were quantified by the 3,3′,5,5′-tetramethylbenzidine (TMB) assay. In order to inhibit the production of toxic oxidants such as hypochlorous acid by MPO each peptide must contain amino acids that possess: i) an N-terminus amino acid #1 (AA1), that is a native amino acid or an artificial amino acid with a basic side-chain such as an amine functional group (e.g. Lysine (K)). ii) Amino acid #2-5 (AA2-5), is a native or artificial amino acid that may contain a polar, non-polar, aromatic ring or hetero-atom containing side-chain that can stabilize a radical species and has specific proximity to the iron/heme of theMPO active site, and can participate in direct radical transfer to the amino acid side-chain. iii) Amino acid #2-5 (AA2-5), is a native or artificial amino acid that also possesses functional groups that interact with any functionality of the overall MPO active site (iron/heme or side-chains of other active site amino acids) through ionic, dipolar, pi-pi, hydrophobic or hydrophilic interactions. These interactions “lock” the peptide into the MPO active site and facilitate radical transfer from MPO to the peptide. iv) Amino acid #2-5 (AA2-5,) is a native or artificial amino acid that also possesses a heteroatom that can further stabilize a free radical. The free radical itself may act as an activated species, and is stable enough to react with proximal proteins, peptides, metabolites or small molecules to effect further change. The free radical is not limited to a single target, but may effect change multiple independent targets. In a non-limiting example, the free radical goes on to form a hetero- or homo dimer via an oxidized —S—S— disulfide (or di-Selenium) linkage. Finally, the more interactions between the peptide and active site of MPO that occurs the lower numerical IC50 value of the peptide inhibitor.

Operationally, an AA(n) (where AA represents different amino acids, and n can be 2-5) peptide is a substrate that is oxidized by MPO to ultimately generate a peptide radical that autoscavenges when it forms a disulfide homodimer. The AA(n) peptide radical may also be scavenged when it forms a disulfide with the thiol of cysteine, glutathione or the thiol of cysteine in another peptide or protein. The free radical itself may act as an activated species, and is stable enough to react with proximal proteins, peptides, metabolites or small molecules to effect further change. The affinity of the AA(n) peptide for the active site of MPO should be higher than the affinity of endogenous MPO substrates (such as chloride and nitrite) in order to prevent MPO from binding chloride or nitrite and oxidizing these ions to hypochlorous acid and nitronium ion, respectively.

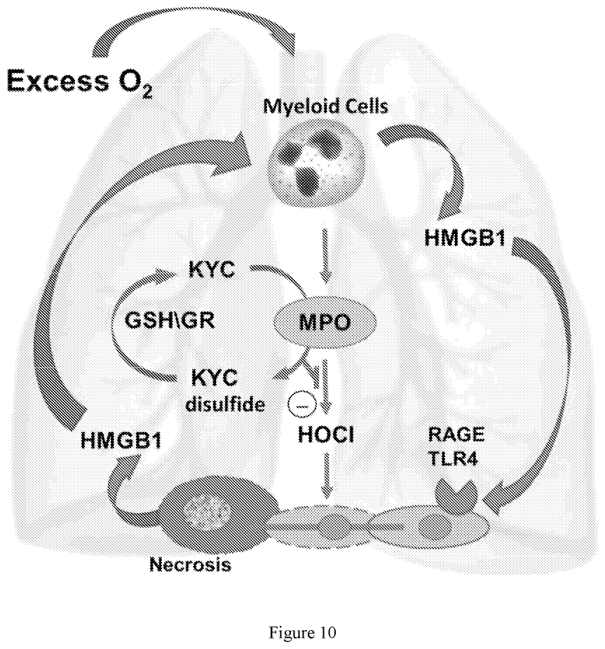

AA₍ₙ₎ peptides compete with MPO's native substrates, such as chloride, and nitrite, etc., and prevents them from entering the active site of MPO. AA₍ₙ₎ competitive inhibition of the binding of these native substrates prevents MPO from generating toxic oxidants such as hypochlorous acid and nitronium ion (FIG. 10). AA₍ₙ₎ peptide inhibition of MPO toxic oxidant production reduces or prevents MPO- and myeloid cell-dependent oxidative damage, myeloid cell activation, and disease progression. The AA₍ₙ₎ peptide is\can be an end-capped molecule such as an N-acetylated and C-amidated tripeptide, in order to reduce proteolytic activity and increases the half-life of the peptide in vivo. The AA₍ₙ₎ peptide is anticipated to have the same reactivity for oxidants as glutathione. In the presence of MPO however, AA₍ₙ₎ peptide is proximal to the iron heme site of activated MPO (Complex I , II or III) and is oxidized by the peroxyl radical that is bound to the iron-heme site to generate a peptide radical species, which when an amino acid containing a heteroatom such as S or Se is also present can form a lower energy thiol radical. In the absence of MPO, the free thiol of cysteine or the heteroatom of a different AA₍ₙ₎ peptide can also directly scavenge free radicals, peroxides and lipid peroxides using essentially the same chemistry as glutathione.

### The KXZ Tripeptide: An Exemplary SCPD

We have demonstrated this phenomenon using an exemplary KXZ tripeptide (in KXZ amino acid #1 (AA₁, K) is a native amino acid or an artificial amino acid (aa) with a basic side-chain such as an amine functional group (e.g., Lysine (K)); Amino acid #2 (AA₂, X) is a native or artificial amino acid that may contain a polar, non-polar, or aromatic amino acid; and Amino acid #3 (AA₃, Z) is a native or artificial amino acid that possesses a heteroatom that can stabilize a free radical that then goes on to form a heteroatom-carbon bond in the form of a homo- or heterodimer of the tripeptide) in a model of bronchopulmonary dysplasia. However, SCPDs can be used to treat a wide variety of diseases and conditions.

An example of a SCPD is any one of the KXZ peptides in the library of peptides as it pertains to MOA in Bronchopulmonary Dysplasia (see example 1 below). KXZ is a tripeptide substrate and inhibitor of the peroxidase enzyme MPO. This enzyme is present in myeloid cells such as neutrophils, monocytes and macrophages. Humans employ MPO primarily as an anti-infective agent that produces the toxic oxidant HOCl.

When KXZ interacts with MPO the tripeptide is reduced (gains an electron in the form of a radical) to form KXZ* (where * represents a free electron radical species) as well as inhibits the production of HOCl. The KXZ* can go on to thiolate proximal proteins of MPO, such as HMGB1, resulting in the loss of pro-inflammatory activity of this protein. Additional proteins such as RAGE, TRL4, NRf2, and KEAP-1 also appear to be regulated by the presence of KXZ/KXZ*.

Such results indicate that KXZ is a systems pharmacology drug. However, the novel mechanism of action requires a distinction from all known current systems pharmacology drugs. In this specific case, KXZ is chemically modified by MPO, whilst effecting MPO activity and function, to subsequently produce KXZ*, and it is the latter that then produces further regulatory effect by thiolation of proximal proteins such as HMGB1.

In order to differentiate the mechanism of action of KXZ from other systems pharmacology drugs, we have labeled this class of compounds as systems chemico-pharmacology drugs. The definition of such a drug is that it predicated on molecular interactions between a single drug and multi-targets present in defined pathways/networks. In addition the parent systems pharmacological agent must react chemically with at least one target to produce a new chemical entity (in this case a radical species (KXZ→KXZ*) that then continues to modulate additional new pathway/network associated targets.

In sum, KXZ peptides in the presence of MPO are a clear example of a SCPD. KXZ peptides are multimodal in function and appear to regulate a number of different proteins, especially when modified by the initial target. Examples of such proteins include, but are not limited to, MPO, HMGB1, RAGE, TRL4, Nrf2, and KEAP1.

### Benefits of SCPDs

A systems chemico-pharmacology drug exemplified by KXZ is designed to both inhibit MPO generation of toxic oxidants and subsequently inhibit other pro-inflammatory peptides/proteins associated with neutrophil mediated inflammation. In addition it is designed to provide the following non-limiting advantageous properties.

First, a single systems chemico-pharmacology drug with multi-target activity should have a more predictable, therefore superior pharmacokinetic (PK) and pharmacodynamic (PD) profile compared to a number of individual drugs administered either as a single drug or in combination.

Second, acute toxicity may be enhanced in more non-selective single/combination therapies.

Third, adverse synergistic effects may be more pronounced in single/combination therapies.

Fourth, the probability of developing target-based resistance to multi-target drugs is statistically lower than is the probability of developing resistance against single-target drugs.

Fifth, the administration of a single systems pharmacology drug results in a more consistent and predictable ADME profile.

Sixth, drug-drug interactions do not exist in a systems chemico-pharmacology regime.

Finally, a single agent binding to multiple targets might be easier to develop, given that the regulatory requirements showing safety/efficacy of a drug combination.

### KXZ Structure

As discussed above, the inventors have discovered that peptide-based inhibitors of peroxidase activity designated as “KXZ” may act as SCPDs in the presence of MPO. Such peptide inhibitors are particularly useful for improving vascular function, decreasing pulmonary inflammation and increasing cardioprotection in a subject.

In view of the inventors' discovery, certain peptide-based SCPDs of the invention (KXZ) have the formula AA₁-AA₂-AA₃. Amino acid #1 (AA₁, K) is native amino acid or an artificial amino acid (aa) with a basic side-chain such as an amine functional group (e.g., Lysine (K)). Amino acid #2 (AA₂, X) is a native or artificial amino acid that may contain a polar, non-polar, or aromatic amino acid. Amino acid #3 (AA₃, Z) is a native or artificial amino acid that possesses a heteroatom that can stabilize a free radical. The free radical itself may act as an activated species, and is stable enough to react with proximal proteins, peptides, metabolites or small molecules to effect further change. The free radical is not limited to a single target, but may effect change multiple independent targets. In a non-limiting example, the free radical goes on to form a heteroatom-carbon bond in the form of a homo- or heterodimer of the tripeptide. A non-limiting example of KXZ is the tripeptide KYC.

In some embodiments, the amino termini may be protected by an acetyl group, and/or the carboxyl termini may be protected by an amide. The disclosure also encompasses peptides comprising retro and retro-inverso analogs of each of the sequences. In retro forms, the direction of the amino acid sequence is reversed. In inverso forms, the chirality of the constituent amino acids is reversed (i.e., L form amino acids become D form amino acids and D form amino acids become L form amino acids). In the retro-inverso form, both the order and the chirality of the amino acids are reversed. A given amino acid reference amino acid sequence and its retro-inverso form are mirror images of each other, and typically have similar functions.

In certain embodiments the peptide can be a cyclic peptide.

In certain embodiments the SCPD is a deuterated (either partially or per-deuterated) compound/peptide.

In certain embodiments, peptides of the invention further contain a protecting group coupled to the amino or carboxyl terminus of the peptide, or a first protecting group coupled to the amino terminus of the peptide and a second protecting group coupled to the carboxyl terminus of the peptide. Possible protecting groups for use in this embodiment include without limitation amide, 3 to 20 carbon alkyl groups, Fmoc, Tboc, 9-fluorene acetyl group, 1-fluorene carboxylic group, 9-florene carboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bz1), 2-chlorobenzyloxycarbonyl (2-Cl—Z), 2-bromobenzyloxycarbonyl (2-Br—Z), Benzyloxymethyl (Bom), t-butoxycarbonyl (Boc), cyclohexyloxy (cHxO), t-butoxymethyl (Bum), t-butoxy (tBuO), t-Butyl (tBu), acetyl (Ac), and Trifluoroacetyl (TFA).

In certain other embodiments, the peptide contains a protecting group coupled to the amino terminal and the amino terminal protecting group is acetyl. In other embodiments, the peptide contains a protecting group coupled to the carboxyl terminal and the carboxyl terminal protecting group is an amide. In still other embodiments, the peptide contains a first protecting group coupled to the amino terminus that is an acetyl, and a second protecting group coupled to the carboxyl terminal that is an amide.

The disclosure also includes pharmaceutical compositions for simultaneously inhibiting peroxidase activity and targeting other disease-related pathways, containing one or more of the peptides described above and a pharmaceutically acceptable carrier. Preferably these compositions are in unit dosage forms such as tablets, pills, capsules, powders, granules, sterile parenteral solutions or suspensions, metered aerosol or liquid sprays, drops, ampules, auto-injector devices or suppositories; for oral, parenteral, intranasal, sublingual or rectal administration, or for administration by inhalation or insufflation. It is also envisioned that the peptides of the present invention may be incorporated into transdermal articles designed to deliver the appropriate amount of peptide in a continuous fashion.

It is not critical whether an inhibitor according to the invention is administered directly to a peroxidase, to a tissue comprising the peroxidase, a body fluid that contacts the peroxidase, or a body location from which the inhibitor can diffuse or be transported to the peroxidase. It is sufficient that the inhibitor is administered to the subject in an amount and by a route whereby an amount of the inhibitor sufficient to inhibit the peroxidase arrives, directly or indirectly at the peroxidase.

For preparing solid compositions such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture for a compound of the present invention, or a pharmaceutically acceptable salt thereof. When referring to these pre-formulation compositions as homogeneous, it is meant that the active ingredient is dispersed evenly throughout the composition so that the composition may be easily subdivided into equally effective unit dosage forms such as tablets, pills and capsules. The tablets or pills of the novel composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium caboxymethylcellulose, methylcellulose, polyvinylpyrrolidone or gelatin.

In certain embodiments, the peptides of the invention will be provided as pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts, which may, for example, be formed by mixing a solution of the peptide according to the invention with a solution of a pharmaceutically acceptable acid such as hydrochloric acid, sulphuric acid, methanesulfonic acid, fumaric acid, maleic acid, succinic acid, acetic acid, benzoic acid, oxalic acid, citric acid, tartaric acid, carbonic acid or phosphoric acid. Furthermore, where the compounds of the invention carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

Suitable dosage levels for the inhibition of peroxidase activity in a human subject (i.e, an effective therapeutic amount to inhibit peroxidase activity) is about 0.01-5000 mg/kg, per day, preferably about 0.1-500 mg/kg per day, and especially about 0.1-50 mg/kg per day.

In another aspect, the invention provides a method of treating a disease or condition in a subject that is associated with excess peroxidase activity. The method includes the step of administering to a subject in need of such therapy one or more of the peptides as described above. In certain preferred embodiments, the subject is a human or a non-human mammal. Preferably, the method includes the additional step of mixing the peptide with a pharmaceutically acceptable carrier before the peptide is administered. In preferred embodiments of the invention, the method is carried out to improve vascular function, decrease pulmonary inflammation, and/or increase cardioprotection in the subject. However, it can be appreciated that the inventive peptides act on a molecular process common to a plethora of medical diseases and conditions. Therefore, the present peptides are envisioned to be useful in treating a wide range of diseases and conditions attributable to aberrant peroxidase activity, including but not limited to, wound inflammation, hypersensitivity, digestive disease, cardiovascular disease, neuronal disease, lung disease, autoimmune disease, degenerative neurological disease, degenerative muscle disease, infectious disease, disease associated with graft transplantation, allergic disease, musculo-skeletal inflammation, and sepsis.

Methods of the invention are further envisioned to be useful in treating hypertension, peripheral vascular disease, pulmonary inflammation, asthma, atherosclerosis, diabetes, persistent pulmonary hypertension, sickle cell disease, neurodegenerative disease, multiple sclerosis, Alzheimer's disease, lung cancer, lupus, ischemic heart disease, Parkinson's disease, Crohn's disease, inflammatory bowel disease, necrotizing enterocolitis, arthritis, polymyocytis, cardiomyopathy, psoriasis, amyotrophic lateral sclerosis, muscular dystrophy, cystic fibrosis, attention deficiency hyperactive disorder, acute lung injury, acute respiratory distress syndrome, flu (including H1N1), heart failure, chemotherapy-induced heart failure, arthritis, rheumatoid arthritis, acute myocardial infarction, traumatic brain injury (TBI), chronic traumatic encephalopathy (CTE), ischemic or hemorrhagic stroke, or bronchopulmonary dysplasia.

Further, the disclosed methods will find use in the promotion of angiogenesis in tissues of a subject, or the promotion of angiogenesis impaired by persistent pulmonary hypertension, peripheral vascular disease or vascular disease in the myocardium in the subject, or the treatment of a disease or condition associated with abnormal, excessive blood vessel development in the subject. The disclosed methods of are additionally useful in treating subjects for the reduction and/or prevention of ischemic injury to a subject's heart following an ischemic event or insult.

The disclosure also encompasses the use of a peptide as described herein for the manufacture of a medicament for inhibiting peroxidase activity and subsequently targeting a second pathway in a subject. Such methods include the steps of (a) providing a peptide as described herein, and (b) mixing the peptide with a pharmaceutically acceptable carrier. As well, the invention encompasses the manufacture and use of medicaments specifically-purposed for treatment of one or more of the diseases/conditions described above.

The following example is offered for illustrative purposes only and is not intended to limit the scope of the invention in any way. Indeed, various modifications of the invention in addition to those shown and described herein will become apparent to those skilled in the art from the foregoing description and the following examples and fall within the scope of the appended claims.

### III. EXAMPLES

### Example 1

### Demonstrating Systems Chemico-Pharmacology Properties of Exemplary KXZ Tripeptide in Bronchopulmonary Dysplasia Model

This example provides “proof of principle” by demonstrating the use of an exemplary KXZ tripeptide to target multiple pathways in a bronchopulmonary dysplasia disease model.

Summary.

Although many consider myeloid cells to play important roles in hyperoxia-induced bronchopulmonary dysplasia (BPD), the role of myeloperoxidase (MPO) is unclear. We hypothesize that MPO increases BPD by inducing an oxidative and inflammatory destructive cycle.

To test this hypothesis Sprague-Dawley neonatal pups were raised in normoxic or hyperoxic (>90% oxygen) environments from day one (P1) until day ten (P10) of life. Hyperoxic neonatal pups were treated each day with either N-acetyl lysyltyrosylcysteine amide (KYC) to inhibit MPO, or phosphate buffered saline from P1 through P10 and then euthanized. Lungs were examined for changes in lung morphometrics, oxidative stress, inflammation, and myeloid cell counts.

Chronic hyperoxia impaired lung development, decreased microvascular and alveolar complexity, and increased lung pathology based on marked increases in MPO+ myeloid cell counts; MPO; 3-chlorotyrosine (Cl-Tyr); 3-nitrotyrosine (NO₂-Tyr); high mobility group box 1(HMGB1); receptor for advanced-glycosylated end-products (RAGE), and, toll-like receptor 4(TLR4). In contrast, KYC treatment reduced MPO+ myeloid cell counts, increased microvascular and alveolar complexity, and decreased MPO, Cl-Tyr, NO₂-Tyr, HMGB1, TLR4, and RAGE levels, and lung pathology.

Taken together, these data indicate that MPO establishes a destructive cycle predominantly mediated by excess oxygen (O₂), myeloid cells, MPO, HOCl, and pulmonary release of HMGB1. Clearly, MPO oxidants play a causal role in propagating this cycle to increase lung pathology since inhibiting MPO toxic oxidant production reduces lung pathology and improves lung development in hyperoxic neonatal rat pups.

Introduction.

Bronchopulmonary dysplasia (BPD) is caused by complications from respiratory distress syndrome of the newborn. BPD affects more than 10,000 infants annually (32), making it the most common pulmonary morbidity of premature infants in the United States (14). Premature neonates have immature lungs that do not function well enough to support life and therefore must be provided respiratory support, such as supplemental oxygen and mechanical ventilation. Both interventions are known to increase oxidative lung injury and BPD (14).

Experimental strategies for treating BPD range from treating with antioxidants to scavenge oxidants (24, 47), supplementing the antioxidant defense system (40), as well as blocking myeloid cell recruitment (2, 3, 15). Since myeloid cell recruitment always precedes BPD (13), inhibiting recruitment should be an effective therapeutic strategy for reducing BPD. Although anti-cytokine-induced neutrophil chemoattractant-1 (CINC-1) antibodies and CXC chemokine receptor-2 (CXCR2) antagonist have both been used to reduce neutrophil infiltration and myeloperoxidase (MPO) release in the lungs of established neonatal rat models of BPD (3, 15) neither approach has progressed towards clinical implementation.

MPO is considered by many to play a causal role in inducing oxidative injury and inflammatory lung disease (4, 10, 17, 28). However, to our knowledge, few if any studies have examined the mechanistic role of MPO in BPD. Recently, we reported that MPO generation of toxic oxidants increases oxidative damage in secondary brain injury in a murine model of stroke (56), neuroinflammation in experimental autoimmune encephalomyelitis (EAE) mice (60), and impaired endothelial- and eNOS-dependent vasodilatation in a murine model of sickle cell disease (61). MPO is a robust peroxidase that has evolved to generate large quantities of free radicals and oxidants to kill invasive bacteria. MPO oxidation of chloride anions (Cl−) produces hypochlorous acid (HOCl), while MPO oxidation of nitrite (NO₂⁻) generates predominantly nitrogen dioxide (NO₂*) (11). As HOCl oxidizes protein tyrosine to form 3-chlorotyrosine (Cl-Tyr) (54), many consider tissue levels of Cl-Tyr to be an index of MPO-dependent oxidative damage (45). In contrast, peroxynitrite and NO₂* are both capable of oxidizing tyrosine to 3-nitrotyrosine (NO₂-Tyr) (19, 42). Accordingly, NO₂-Tyr is considered a footprint of both forms of nitrosative stress. Data supporting the idea that MPO oxidants are involved in disease pathology are often indirect, based on relative differences in the levels of Cl-Tyr and/or NO₂-Tyr in lung tissues before and after treatment or from comparison of biomarker levels in wild-type and MPO-knockout mice (27, 28, 60). Selective attenuation of MPO activity by either transgenic knockout or selective inhibition with non-toxic small molecules are both logical strategies for determining the extent of MPO's contribution to BPD pathology.

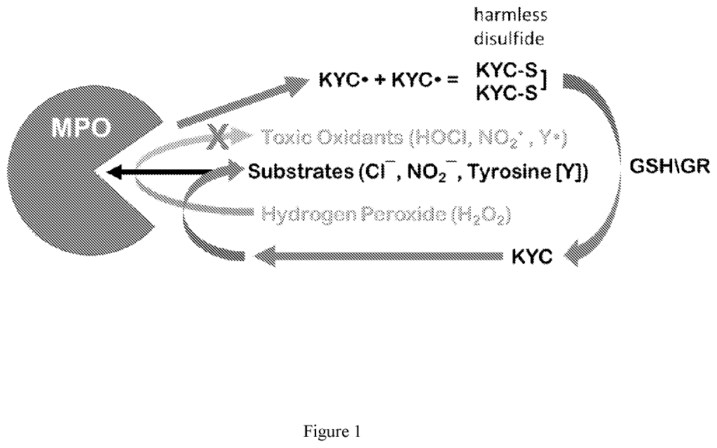

MPO knockout rats are not currently available, hence we investigated the role of MPO in hyperoxia-induced BPD in neonatal rat pups by inhibiting MPO oxidant production with N-acetyl lysyltyrosylcysteine amide (KYC). KYC is a novel tripeptide inhibitor of MPO toxic oxidant production that reduces MPO-dependent secondary brain injury in a murine model of stroke (56, 57), neuroinflammation in a murine model of multiple sclerosis (58, 60), dose-dependently protects endothelial cell cultures from MPO-mediated cell death (59), and improves endothelial- and eNOS-dependent vasodilatation in an established murine model of sickle cell disease (61). Mechanistically, KYC is unique in its ability to reduce MPO toxic oxidant production because it is a substrate that reduces the iron heme of MPO to ground state and in so doing shuttles MPO oxidants directly into the glutathione (GSH) system (FIG. 1).

Here, we present data showing KYC inhibition of MPO breaks the proposed destructive cycle between MPO, HMGB1, and myeloid cell recruitment to reduce oxidative damage and inflammation in an established neonatal rat pup model of chronic hyperoxia-induced BPD model.

Materials and Methods.

Peptide Synthesis: KYC was synthesized using Fmoc [N-(9-fluorenyl)methoxy-carbonyl] chemistry, prepared and purified as an acetate salt by Biomatik USA, LLC (Wilmington, Del.), as previously described (56, 60). Trifluoroacetic acid (TFA) in tripeptide preparations was reduced by the Peptide Core (Blood Research Institute, Medical College of Wisconsin) by dissolving KYC in distilled water containing 6 mM HCl followed by lyophilization (2-3×). TFA content in peptide preparations was quantified by NMR (30). TFA in KYC preparations was reduced to <0.01% prior to being used for experiments. All other chemicals were obtained from Sigma-Aldrich (St. Louis, Mo.).

Antibodies: Rabbit antibodies for myeloperoxidase heavy chain (MPO, sc-33596) and for TLR4 (sc-10741) were from Santa Cruz Biotechnology (Dallas, Tex.). Rabbit antibody for Cl-Tyr (HP5002) was from Hycult Biotech (Plymouth Meeting, Pa.). Rabbit antibody for NO₂-Tyr (N0409) was from Millipore-Sigma (St. Louis, Mo). Chicken antibody for HMGB1 (326052233) was from SHINO-TEST Corporation (Kanagawa, Japan). Rabbit antibody for RAGE (GTX23611) was from GeneTex (Irvine, Calif.). Mouse antibodies for PECAM-1/CD31 (ab24590) and for rat endothelial cell antigen-1 (RECA-1, ab9774) were from Abcam (Cambridge, Mass.).

Rats and Experimental Protocols: Sprague-Dawley rats were obtained from Harlan Laboratories (Madison, Wis.) and pregnancy was achieved naturally in our animal facility. Animal protocols were submitted to, and approved by, the Medical College of Wisconsin's Institutional Animal Care and Use Committee and conformed to NIH Guide for the Care and Use of Laboratory Animals. The animals were housed in barrier cages with a 12-h dark-light cycle and were given free access to chow and water. The dam and pups were placed in a cage in either room air (normoxia) or >90% oxygen chamber (hyperoxia) from postnatal day 1 to day 10 (P1-P10) to generate BPD as previously reported (25, 51). Oxygen concentrations were continuously monitored with an oxygen sensor (Reming Bioinstruments Co., Redfield, N.Y.).

Since the number of neonatal rat pups per pregnant dam limits the number of experimental conditions that can be compared, we modified our standard experimental design protocol. The first experiment was designed to determine if hyperoxia increased BPD, while the second was designed to determine if inhibiting MPO oxidant production reduced BPD. At least three sets of neonatal pups from three different dams were used for each experiment. Pups were fed ad libitum from nursing dams. Pups were weighed and inspected daily in room air for less than ten minutes. Nursing dams were switched daily to avoid the impact of nutritional status. Experience has taught us that severity of hyperoxia-induced BPD is litter dependent. To minimize litter differences, we determined the effects of hyperoxia vs. normoxia on BPD in neonatal pups by mixing pups from the litters and randomly reallocating pups with nursing dams.

To determine the effects of KYC on BPD in hyperoxic neonatal pups we randomly assigned pups to the phosphate buffer solution (PBS) control group or the KYC treatment group. The dose of KYC (5 mg/kg twice per day) was chosen based on experience treating chronic inflammation in other disease states (58, 60, 61). KYC was injected i.p., starting on P2 to half of the randomly assigned pups in each litter using a sterile insulin syringe fitted with a 30G needle (Beckon Dickinson, New York, N.Y.) until P10. Equal volumes of PBS were injected i.p. into the remaining half of the pups as an injection control. Pups were euthanized on P10 with carbon dioxide and lungs removed en bloc. A small cut was created on the left atrium and ice-cold normal saline was gently infused through the right ventricle to flush blood from the lungs before inflation for histology or snap-frozen in liquid nitrogen for protein studies. Pups from at least three litters were used in each experiment. Survival rates were determined by plotting the deaths of hyperoxic neonatal rat pups treated with either PBS or KYC on the postnatal day of discovery. Kaplan-Meier survival curves were plotted and analyzed from Kaplan-Meier tables constructed in GraphPad Prism version 7.00 for Windows (GraphPad Software, La Jolla, Calif.).

Immunohistochemistry and Immunocytochemistry: After cannulating the trachea with an Instech Solomon (20G) stainless steel feeding tube (Plymouth Meeting, Pa.), the lungs were inflated with 10% neutral buffered formalin at 25 cm-H2O (2.4 kPa) for 1 h. Lungs were removed after the trachea were securely tied with surgical silk to maintain the pressure, and perfusion fixed with an additional aliquot of 10% neutral buffered formalin for 24 h before paraffin embedding. Lung sections (5 μm) were mounted on SuperFrost plus-coated slides (Denville Scientific, Metuchen, N.J.). Slides were deparaffinized and sections stained with hematoxylin and eosin (H&E). Histology images were captured with a mounted digital camera using an Olympus IX 51 microscope and a 10× objective. Inflammatory cells, myeloid cells including neutrophils, monocytes, and macrophages, were stained with MPO antibody (1:200) overnight at 4° C. and HPR-conjugated anti-rabbit antibody (1:1000) at room temperature for one hour then visualized by diaminobenzidine to generate a dark-brown color. Blood vessels were stained with RECA-1 and visualized with horseradish-conjugated secondary antibody and diaminobenzidine. The average of three sections per pup, and five counts per section (15 counts/pup) was used for statistical analysis. Quantified data were obtained and entered into the record using predetermined codes by one of the coauthors who was not involved in taking the images.

Morphometric Analysis: The mean linear intercept (MLI), or chord length, was used as a method to estimate the volume-to-surface ratio of acinar airspaces whereas radial alveolar count (RAC) and secondary septa were investigated to study the complexity of lung structure (21). Ten equally spaced horizontal lines were drawn on each picture, and the number of intercepts through the alveolar wall was counted. MLI was obtained by the number of times the traverses are placed on the lung times the length of the traverse then divided by the sum of all the intercepts. For the RAC, a line from the center of the respiratory tract perpendicular to the nearest connective tissue septum was drawn, and alveoli intercepting with the line were counted. For measurement of secondary septa, elastin was stained with resorcin fuchsin and Van Gieson's solution.

Immunoblot Analysis: Whole lung lysates were obtained by homogenizing in MOPS buffer (20 mM 3-N-morpholino-propanesulfonic acid, 2 mM EGTA, 5 mM EDTA, 30 mM NaF, 10 mM β-glycerophosphate, 10 mM Na pyrophosphate, 2 mM Na orthovanadate, 1 mM PMSF, 0.5% NP-40, 1% protease inhibitor cocktail, and 1% phosphatase inhibitor cocktails 2 and 3, pH 7.0) by Bullet Blender (Next Advance, Inc., Averill Park, N.Y.). For protein immunoblots, 30 μg of protein lysate was separated by SDS-PAGE, transferred to nitrocellulose membranes (0.22 μm), and then probed with the appropriate primary antibodies overnight at 4° C. on a continuously rocking platform. Signals were generated after incubation with horseradish peroxidase-conjugated goat anti-rabbit or anti-mouse IgG (1:8,000) using Western Bright ECL Chemiluminescent HRP Substrate (Advansta Inc., Menlo Park, Calif.). Integrated optical density (IOD) was calculated using ImageJ software and normalized to β-actin, a loading control.

Glutathione Reductase Reduction of KYC Homo- and Hetero-disulfides: Disulfide formation was induced by incubating either KYC alone (2.2 mM) or KYC with equal concentration glutathione (GSH) in PBS (pH 7.4) with shaking overnight at room temperature. The reducing conditions used to assess glutathione reductase (GR) activity on KYC-KYC and KYC-GSH disulfides were carried out using previously established methods (12, 53). Mixed KYC-KYC and KYC-GSH disulfides were incubated with 3.8 μM GR (Thermo-Fisher, Waltham, Mass.) in a 100 mM potassium phosphate buffer (pH 7.5) containing 2 mM EDTA, and 1 mM NADPH for 1 hr. at room temperature with shaking. The reaction was halted by snap freezing at −80° C. Negative controls omitted either GR or NADPH. Samples were analyzed by HPLC (Beckman, Brea, Calif.) at the Blood Research Institute of Wisconsin Protein Core Lab. Samples were diluted 1:1 in 0.1% TFA, loaded on a Jupiter Proteo column (C-12 surface, dimensions: 4.6×50 mm, 4 μm particle size, Phenomenex, Torrence, Calif.) and run with a gradient (solvent B into solvent A) of 2-40% over 10 min with a flow rate of 1 ml/min and read using a UV detector at 220 nm. Solvent A was 0.08% TFA in HPLC grade H2O and solvent B was 0.1% TFA in HPLC grade acetonitrile. Unless otherwise indicated chemicals were obtained from Sigma-Aldrich (St. Louis, Mo.).

Statistics: Representative images of lung sections and immunoblots are presented in the figures. Data from lung sections and immunoblots from the samples were analyzed for statistical differences using GraphPad Prism version 7.00 for Windows (GraphPad Software, La Jolla, Calif.). Quantitative morphometric and cell count data are presented as mean±SD scatter-plots adjacent to the representative images. Significant differences between groups were determined by either student's t-test or unpaired Mann-Whitney U test, depending on distribution. A p-value<0.05 was considered statistically significant.

Results.

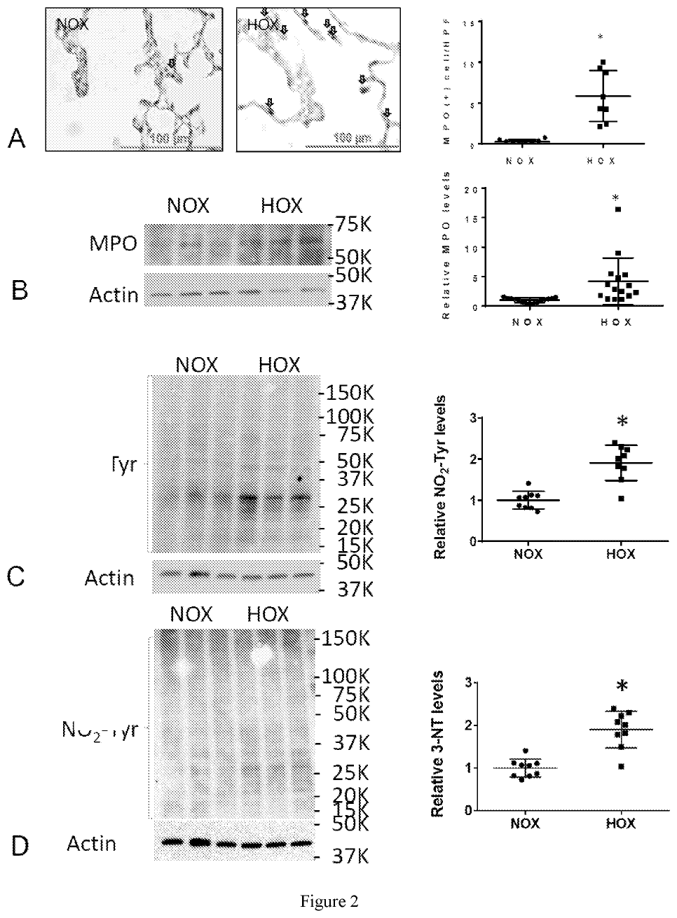

Effects of Hyperoxia: Chronic hyperoxia increased the number of MPO (+) myeloid cells in the lungs from neonatal pups compared with the number in lungs from normoxic pups (brown stained cells, FIG. 2A). Counts of brown-stained cells revealed that hyperoxia increased MPO positive (+) myeloid cell recruitment by 10- to 20-fold. Immunoblots for MPO confirmed histology findings. Quantification of band intensities showed hyperoxia increased MPO protein in lungs of neonatal pups by 5-10-fold (FIG. 2B). Immunoblots for Cl-Tyr and NO2-Tyr revealed that hyperoxic lungs experienced greater MPO-dependent oxidative and nitrosative stress than normoxic lungs (FIGS. 2C and 2D, respectively). Image analysis of the immunoblots revealed that hyperoxia increased Cl-Tyr formation by approximately 170% and NO2-Tyr formation by approximately 90%. These data demonstrate that chronic hyperoxia increases myeloid cell recruitment and infiltration into and MPO-dependent oxidative damage to neonatal lungs.

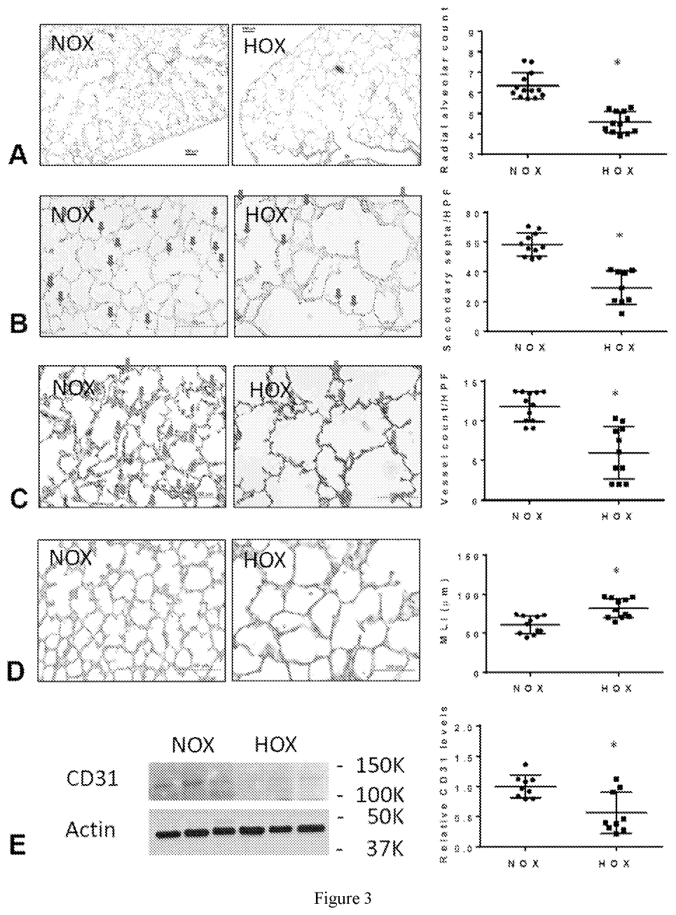

Effects of Hyperoxia on Lung Morphometrics: Hyperoxic lungs had decreased RAC, secondary septa and blood vessel counts by approximately 27%, 52%, and 52%, respectively, and increased MLI values by approximately 40% compared with normoxic lungs (FIG. 3A-D). As many, if not all, of these architectural features in developing lungs are influenced by blood vessel growth, lysates of lung homogenates were immunoblotted for CD31 to assess the levels of endothelial cell proliferation in the developing lung. The immunoblots showed that hyperoxia decreased CD31 in lung lysates by approximately 47% (FIG. 3E). These findings are consistent with the immunohistochemistry studies showing that chronic hyperoxia decreased the number of blood vessels and the complexity of vessel architecture in neonatal lungs.

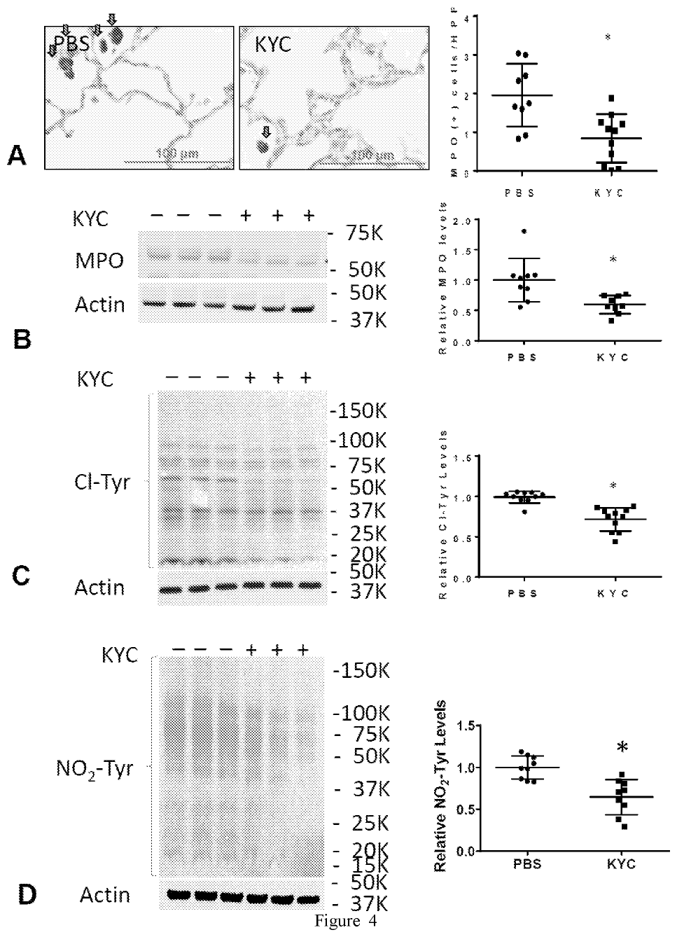

Effects of KYC on Hyperoxia-induced Myeloid Cell Recruitment, MPO, and Oxidative Stress: KYC reduced the number of MPO+ myeloid cells in hyperoxic lungs (brown cells in FIG. 4A). MPO immunoblots show that KYC treatment reduced MPO protein (FIG. 4B) as well as Cl-Tyr and NO2-Tyr formation in hyperoxic lungs (FIGS. 4C and 4D, respectively). Cell counts, and image analysis of band size and intensity both indicate that KYC reduced myeloid cell recruitment by approximately 50%, MPO protein by approximately 50%, Cl-Tyr formation by approximately 27%, and NO₂-Tyr by approximately 32% in hyperoxic lungs. Taken together, data in FIGS. 2-4 suggest that chronic hyperoxia markedly impaired lung development in the neonatal rat pups by a myeloid- and MPO-dependent mechanism.

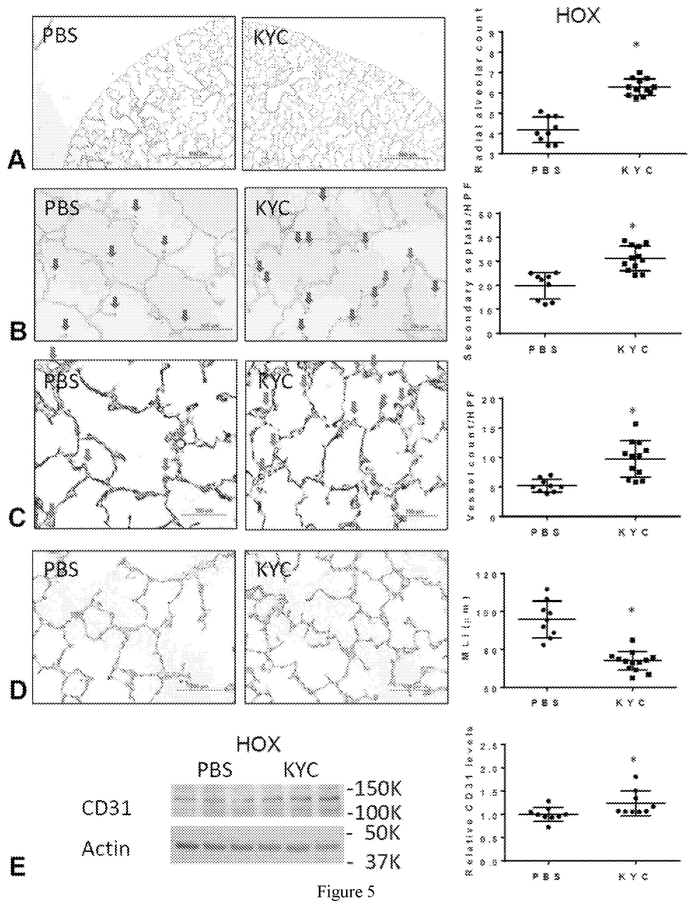

Effects of KYC on Hyperoxia-induced BPD: If MPO plays a causal role in the mechanisms by which hyperoxia induces BPD, then KYC, which inhibits MPO toxic oxidant production but not peroxidase activity (59), should reduce hyperoxia-induced changes in lung architecture. Morphometric analysis shows that KYC treatment of hyperoxic pups increased RAC, secondary septa and blood vessel counts by approximately 35%, 55%, and 92%, respectively, while decreasing MLI (approximately 22%) in the lungs of the hyperoxic pups compared with the stunted development of lung architecture in PBS-treated hyperoxic pups (FIG. 5A-5D). Immunoblots for CD31 confirm that KYC increased endothelial cell proliferation (approximately 23%) in the lungs of hyperoxia pups (FIG. 5E). The increase in CD31 levels determined by immunoblotting confirms the immunohistochemistry showing that KYC increased microvascular proliferation and alveolar complexity, which are essential for improving lung architecture and decreasing BPD in hyperoxic pups.

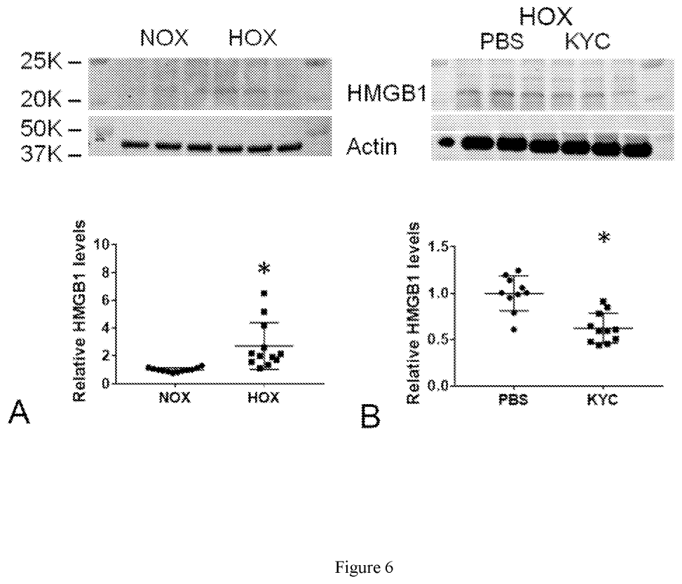

Effects of Hyperoxia and KYC on Lung HMGBJ: Immunoblots show that chronic hyperoxia increased HMGB1 levels in lung homogenates by 170% compared with HMGB1 levels in lung homogenates from normoxic pups (FIG. 6A). In contrast, HMGB1 levels in lung homogenates from hyperoxia pups treated with KYC decreased by approximately 38% compared with the levels in lung homogenates from hyperoxic pups treated with PBS (FIG. 6B). Taken together, these data (FIGS. 3-6) suggest that KYC inhibition of MPO oxidant production decreased oxidative damage and reduced HMGB1 levels in the lungs of neonatal rat pups chronically exposed to hyperoxia.

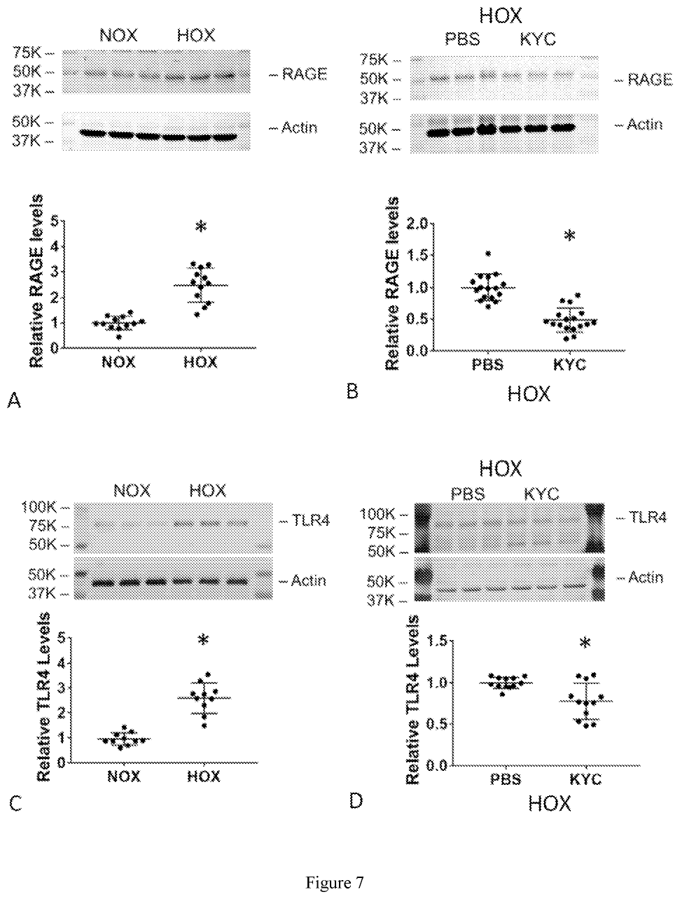

Effects of Hyperoxia and KYC Treatment on RAGE and TLR4 Expression in Lungs of Hyperoxic Neonatal Rat Pups: Oxidative stress and inflammation are reported to increase pulmonary expression of RAGE and TLR4 (16, 29). Lung homogenates were examined for changes in RAGE and TLR4 expression to determine if MPO-dependent oxidative stress modulates. FIG. 7 shows the effects of hyperoxia and KYC treatment on RAGE and TLR4 expression in neonatal rat pup lungs. Hyperoxia increases expression of RAGE by approximately 130% (FIG. 7A). KYC treatment of hyperoxic neonatal rat pups reduces RAGE expression by 60% (FIG. 7B). Hyperoxia increases TLR4 expression in neonatal rat pups by approximately 160% (FIG. 7C), while KYC treatment of the hyperoxic neonatal rat pups reduces TLR4 expression by approximately 22% (FIG. 7D). These data show that MPO oxidants play important roles the mechanisms by which supplemental oxygen increases expression of these innate immune receptors to increase pulmonary inflammation.

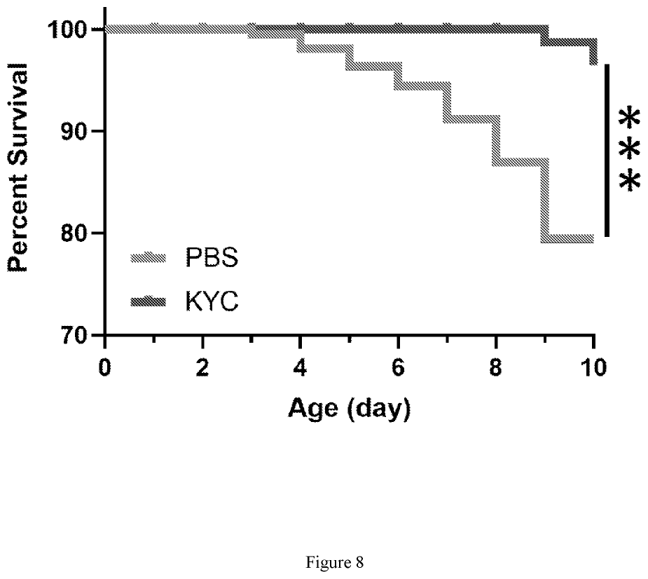

Effects of KYC on Survival of Neonatal Rat Pups Exposed to Hyperoxia. A total of 66 neonatal rat pups were used in this study. The survival rate for PBS-treated neonatal rat pups exposed to hyperoxia was 82.8% at P10 (FIG. 8, red curve). In contrast, the survival rate for KYC-treated neonatal rat pups exposed to hyperoxia was increased to 97.3% at P10 (FIG. 8, blue curve). These data are consistent with the idea that inhibiting MPO reduces oxidative damage to lungs and improves lung architecture. It is important to note that KYC did not induce any sign of over toxicity during the course of treatments. Such data are consistent with previous reports that KYC does not induce any signs of over toxicity in a variety of mouse models of vascular and neurological disease (56-61).

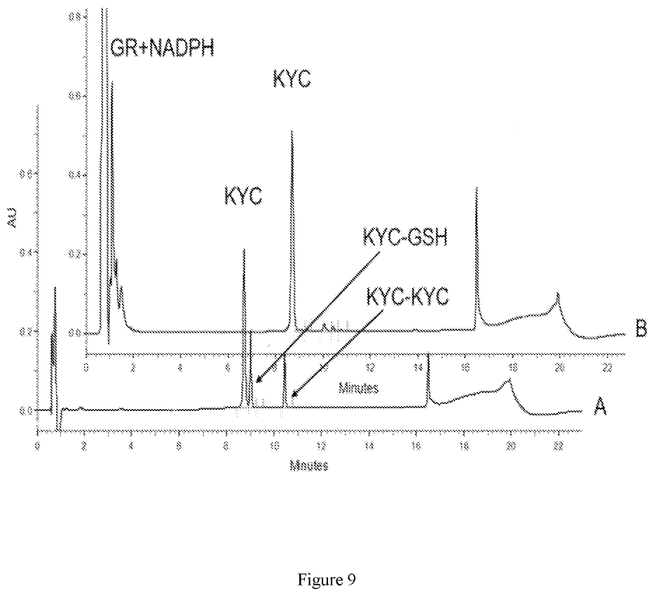

Effects of Glutathione Reductase on Reduction of KYC-KYC Homo-disulfide and KYC-GSH Hetero-disulfide to KYC Monomer: One of the mechanisms by which KYC is hypothesized to reduce MPO-dependent oxidant production is by forming KYC homodimers (KYC-KYC) that can be reduced to an active KYC monomeric inhibitor by GSH (59). GSH homodimer (GS-SG) can be reduced to monomeric GSH by glutathione reductase (GR) (43). GSH homodimers can also undergo thiol exchange to form mixed heterodimers (GS-SR). To determine if KYC homo-disulfide and KYC-GSH hetero-disulfide are reduced by GR, KYC-KYC homodimer and KYC-GSH heterodimer were incubated with a GR\NADPH reaction mixture and KYC monomer formation determined by HPLC as before (59). FIG. 9 shows that the GR+NADPH mixtures reduce KYC-KYC and KYC-GSH to KYC monomers. As GR+NADPH mixture does not reduce KYC homodimers to KYC monomers (data not shown), these data suggest that GSH thiol exchange is required for GR to be able to reduce KYC homo-disulfides to an active KYC monomer for continued inhibition of MPO toxic oxidant production.

Discussion.

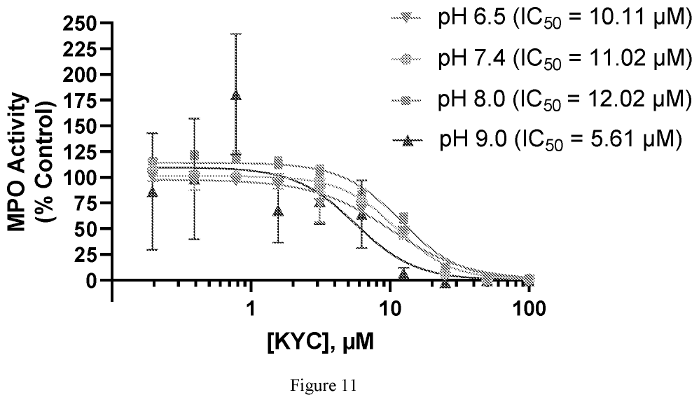

Cycle of Destruction: Our findings demonstrate that hyperoxia increases BPD in neonatal rat pups by a destructive cycle containing at least five basic components, namely; myeloid cells, excess oxygen (O₂), MPO, toxic oxidants (i.e., HOCl), and HMGB1 (FIG. 10, red cycle). Circulating myeloid cells enter a lung that is inflamed by chronic supplemental oxygen. After arrival, the myeloid cells adhere, become activated and release MPO, a major enzymatic source of toxic oxidants, specifically HOCl. This MPO product is a potent oxidant that damages the lung causing the release of HMGB1 protein. HMGB1 is a damage-associated molecular pattern (DAMP) molecule with chemotactic and cytokine-like properties that accelerates myeloid cell recruitment and increases RAGE and TLR4-dependent inflammation. Our data show that this series of events promotes a cycle of myeloid cell recruitment, oxidative lung damage, and HMGB1 release to induce BPD (FIG. 11, red cycle). Support for MPO and HMGB1 playing a central role in this cycle of destruction to induce BPD comes from data showing that treating hyperoxic neonatal rat pups with KYC decreases both MPO and HMGB1 while markedly improving neonatal rat pup lungs. KYC is a non-toxic, end-capped tripeptide that was designed to be an MPO substrate that promotes non-productive peroxidase activity, meaning the peroxidase consumes H₂O₂ without generating toxic oxidants (FIG. 1). We also show that KYC inhibits MPO dependent toxic oxidant production, which breaks the cycle of destruction (FIG. 10, blue cycle).

The destructive cycle induced by hyperoxia is characterized by increased myeloid cell recruitment and MPO release, as seen in the comparative data of FIG. 2. The combined effects of oxidative stress and inflammation propagate the cycle. Immunoblots for Cl-Tyr and NO₂-Tyr show that chronic hyperoxia increases oxidative damage to neonatal lungs (FIGS. 2C and 2D) while immunoblots for HMGB1 (FIG. 6A) show that chronic oxidative damage to the lung increases inflammation by increasing RAGE and TLR4 expression (FIGS. 7A and 7C). HMGB1 clearly promotes myeloid cell recruitment, increases vascular inflammation (5, 6, 9, 35), and increases BPD in hyperoxic full term neonatal C57Bl\6 pups (55). The fact that these changes take place in hyperoxic lungs characterized by fewer alveolae, secondary septa, vessels, and increased MLI counts (FIG. 3A-3D), supports the idea that the hyperoxia-induced cycle of oxidative stress and inflammation is a powerful mediator of impaired lung development. Further support for the cycle impairing lung development comes from immunoblots showing that CD31, a direct readout of vascularization, is decreased in the lungs of hyperoxic neonatal pups (FIG. 3E).

We previously reported that KYC is a highly selective inhibitor of MPO (59), and as such, can serve as a unique probe for determining the extent to which MPO is involved in disease, including BPD. It is noteworthy that KYC treatment reduces all the biomarkers for oxidation, inflammation, and improves lung structure (FIGS. 4 and 5) indicating that MPO-derived oxidants increased BPD in hyperoxic neonatal rat pups. Our data show that KYC reduces the number of MPO+ myeloid cells, as well as MPO content in hyperoxic lungs (FIG. 4A and 4B). As reductions in myeloid cell counts and MPO content are associated with decreased oxidative damage and inflammation, any decrease in levels of Cl-Tyr and NO₂-Tyr in the lungs of hyperoxic neonatal rat pups can be interpreted as a sign of repair and regeneration (52) (FIGS. 4C and 4D). If MPO-derived toxic oxidants are responsible for the loss of lung architecture in BPD, then any increases in alveolar counts, secondary septa, blood vessels, and decrease in MIL counts in hyperoxic neonatal rat pups treated with KYC can be interpreted as the extent to which MPO mediates lung injury in BPD (FIG. 5A-5D). The improvements in lung structure that occur in KYC treated hyperoxic neonatal rat pups demonstrate the degree to which KYC normalizes and/or restores endothelial cell proliferation in lungs of hyperoxic neonatal rat pups. Further support for a direct role for MPO in BPD comes from immunoblot data showing KYC treatment increases CD31 expression in the lungs of hyperoxic neonatal rat pups (FIG. 5E).

Hyperoxia increases pulmonary inflammation by a variety of mechanisms. HMGB1 (18, 41), RAGE (44), as well as TLR4 (7, 33) have all been reported to increase pulmonary inflammation in hyperoxic animals. As mentioned above immunoblots show that HMGB1, RAGE and TLR4 levels are all increased in the lungs of hyperoxic neonatal rat pups (FIGS. 6A, 7A, and 7C, respectively). In contrast, KYC treatment of hyperoxic neonatal rat pups reduced all three mediators of pulmonary inflammation (FIGS. 6B, 7B, and 7D, respectively). Taken together these data demonstrate three things about the cycle illustrated in FIG. 11. First, the cycle is a causal factor in BPD. Second, the cycle is propagated by MPO-derived toxic oxidants, such as HOCl, and HMGB1, which is released from the damaged tissues. Third, inhibiting MPO toxic oxidant production can disrupt this cycle of destruction.

Potential Use of KYC in BPD: One of the potential benefits in thinking that the cycle of destruction is a causal mediator in BPD onset and progression is it provides focus for treatment strategies. Since KYC effectively inhibits MPO-dependent HOCl production and HMGB1 expression and promotes H₂O₂, consumption it may be useful for treating BPD in neonates. Support for this idea comes from data showing that not only does MPO and HOCl play causal roles in the oxidative mechanisms mediating BPD but that inhibiting MPO reduces lung pathology, improves lung architecture, and increases alveologenesis, and vasculogenesis in hyperoxic neonatal rat pups. Inhibiting MPO toxic oxidant generation should be more effective for reducing oxidative damage to the lung because inhibiting oxidant production at its enzymatic source is more effective than trying to scavenge oxidants after they have been formed. The importance of this fundamental concept is underscored by the fact that after HOCl is generated, it is fully capable of oxidizing biomolecules such as membrane phospholipids and lipids to secondary chlorinated lipids that possess extremely cytotoxic properties (23). Our findings illustrate the archetypal principle that inhibiting oxidant generation at its biological source is the most efficient method for preventing oxidative damage.

Another potential advantage in inhibiting oxidant generation compared to intervening downstream (e.g. HMGB1) is highlighted by the following consideration. Yu and colleagues (55) reported on the effectiveness of a polyclonal antibody against HMGB1 in BPD. They reported that survival rates of mouse pups exposed to 85% O₂ increased from ˜75% to ˜91% (P10) after treatment with anti-HMGB1 antibody. In contrast, KYC increased survival of hyperoxic (>90%) neonatal rat pups from ˜83% to ˜98% at P10. While anti-HMGB1 antibodies are effective at reducing myeloid cell recruitment, existing myeloid cells in the hyperoxic lung are still be able to release MPO, produce HOCl, generate H₂O₂, and therefore cause lung injury. In contrast, KYC not only inhibits MPO HOCl production but is a substrate that causes MPO to consume H₂O₂ without generating HOCl. Reduced HOCl production, results in less oxidative damage and as shown here lower levels of HMGB1. While both agents are likely reducing oxidative stress and myeloid cell recruitment, physiologically it appears KYC improves chances for survival more than anti-HMGB1 antibodies.

Previously we reported that when MPO oxidizes KYC, the resultant oxidized product is a KYC radical that auto-scavenges by forming a KYC homo-disulfide (59). However, additional studies suggest that the formation of a disulfide may not be the end of KYC's role as an inhibitor of MPO. As a simple homo-disulfide, KYC disulfide is capable of undergoing thiol exchange reactions and/or direct reductions to a KYC monomer by physiological concentrations of GSH (59). To better understand how KYC modifies thiol cell biology, we determined if glutathione reductase (GR) reduces KYC homo- and hetero-disulfides. Our studies showed that while GR cannot reduce KYC homodimers in the presence of NADPH alone (data not shown), it can reduce KYC homo- and hetero-disulfides in the presence of GSH and NADPH (FIG. 9). These findings extend our previous observations showing that GSH reduces KYC homodimers to KYC monomers. Taken together, these in vitro findings begin to explain why KYC is so effective at inhibiting MPO toxic oxidant production. As a substrate, KYC promotes non-productive MPO peroxidase activity, meaning that MPO oxidation of KYC promotes H₂O₂ consumption while generating oxidants that autoscavenge (FIGS. 1 and 10). KYC's ability to convert MPO into a quasi-catalase may reduce oxidative stress more than inhibiting MPO activity alone, because inhibiting MPO only blocks HOCl production and has no effect on the H2O2 that is generated by activated myeloid cells and uncoupled mitochondria. In this way, KYC acts both as an “antioxidant” and a shuttle for MPO by scavenging oxygen radicals bound to MPO's iron heme site and shuttling the resultant KYC radical into the GSH pathway by forming a KYC disulfide, which can be reduced to active KYC monomers via GSH and GR. As MPO oxidation of KYC also promotes H₂O₂ consumption, such a dual functioning inhibitor should reduce oxidative stress more than inhibitors that only inhibit MPO peroxidase activity. To the best of our knowledge these are the first studies to show that inhibiting MPO toxic oxidant production reduces BPD and improves lung development in an established animal model of BPD.

Although the association of neutrophils with neonatal BPD was reported over 35 years ago in 1983 (34), the role that MPO played in BPD has largely been ignored. Consequently, major gaps in our knowledge exist concerning MPO's role in BPD. Support for this point comes the BPD literature itself, which although excellent reviews for BPD exist, their discussion of MPO's role in BPD is sparse to nonexistent (1, 26, 39, 49). When it comes to standard journal reports, most studies use MPO as a biomarker of neutrophil and myeloid cell recruitment, not as a major mechanism of acute or chronic lung injury in BPD. As a result, investigators have focused on inhibiting myeloid cell recruitment and infiltration rather than targeting MPO. Relegating MPO to biomarker status also influences how one develops therapeutic strategies to inhibit BPD. For example, melatonin and vitamin E are broad-spectrum antioxidants that are capable of scavenging oxidants and free radicals although both have been shown to inhibit BPD in animal models and improve outcomes in clinical studies with mixed results (20, 38, 40, 48). Although melatonin has been used to inhibit MPO, since it is classified as a general antioxidant, its ability to reduce BPD cannot be attributed solely to inhibiting MPO.

Conclusion: Chronic hyperoxia increases BPD in neonatal rat pups by a 5-component destructive cycle: 1) excess O₂, 2) adherent and recruited myeloid cells, 3) MPO, 4) toxic oxidants (i.e., HOCl), and 5) HMGB1 release. As MPO peroxidase activity plays a central role in this cycle, inhibiting MPO should be an effective strategy for reducing the oxidative damage associated with BPD as well as improving lung development in at-risk premature neonates receiving supplemental oxygen.

In 2014, Berkelhamer and Farrow suggested that progress in treating BPD would require the development of novel antioxidant agents that target multiple systems of oxidants (8). Such a statement implies that a systems biology approach should be used to treat BPD, which to our knowledge does not exist. From the studies here, we learned that KYC effectively reduced inflammation and restored development in hyperoxic neonatal lungs. The breadth and depth of KYC's antioxidant and anti-inflammatory effects on hyperoxic neonatal lungs underscores the importance of HOCl-dependent oxidative damage to BPD pathology. Accordingly, inhibiting HOCl production may restore homeostatic balance to multiple cells and systems in BPD simply by preventing the formation of secondary chlorinated phospholipids and lipids that others have shown are toxic and have severe pathological effects (23, 36, 37).

### Example 2

### Demonstrating Systems Chemico-pharmacology Properties of Exemplary AA₍ₙ₎ Peptides

Background and reference data for our new discoveries involving AA₍ₙ₎ peptides are presented herein. Note that N-acetylated, C-amidated AA₍ₙ₎ inhibition of MPO is amino acid sequence dependent and the MOA can change as a function of pH.

i). KYC Peptide

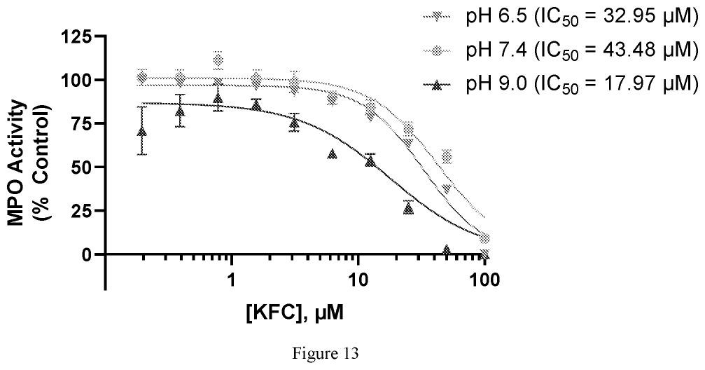

The peptide KYC inhibits MPO employing three of the four attributes of individual amino acids described above. It contains i) an N-terminus amino acid #1 (AA₁), with an amine functional group (e.g. Lysine (K)). ii) Amino acid #2 (AA₂), contains an, aromatic ring side chain (Tyr) that can stabilize a radical species. iii) Amino acid #3 (AA₃,) contains a heteroatom (the sulfhydryl sulfur of Cys) that can further stabilize a free radical. The free radical itself may act as an activated species, and is stable enough to react with proximal proteins, peptides, metabolites or small molecules to effect further change. The free radical is not limited to a single target, but may effect change in multiple independent targets. In a non-limiting example, the free radical goes on to form a hetero- or homo dimer via an oxidized —S—S— disulfide linkage. The changing of pH has limited effect on the IC₅₀ values, as shown in the MPO v KYC (FIG. 13).

ii) KWC Peptide

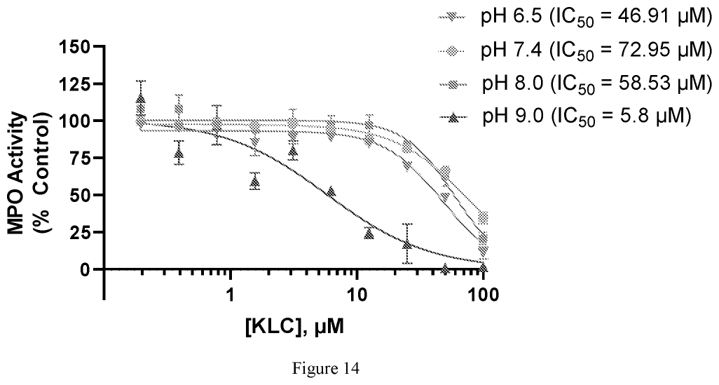

A model peptide such as KWC, does indeed inhibit MPO toxic oxidant production, as shown in FIG. 14 for MPO v KWC. This peptide also fulfills three of the four criteria described above in the KYC data section, but is also much more pH dependent. It is noteworthy the interaction of the peptide functionality directly interacting with the iron/heme of MPO is lacking. Whereas the more general interaction of the indole aromatic ring with the heme porphyrin aromatic system, resulting in less optimal IC₅₀ values observed in the KWC IC₅₀

iii) KFC

The significant higher IC₅₀ values for KFC reinforce the importance of a direct interaction with the iron/heme of MPO (where complex I, II and III all reside). The structure difference between KYC and KFC is simply the hydroxyl group of the aromatic ring (KYC) is removed in KFC, otherwise the peptide structures are identical. This removal results in a ˜four-fold drop of inhibition by KFC versus KYC, as seen in FIG. 13 showing the IC₅₀ curve for KFC.

iv) Different MOA-KLC Peptide

KLC peptide data (see FIG. 14) indicates a completely different MOA for MPO toxic oxidant inhibition compared with KYC. In the case of KLC at pH 9.0, the IC₅₀ is comparable to KYC, whereas at other pH values of 6.5, 7.4 and 8.0 KYC IC₅₀ values are superior, denoting greater inhibitory effects. At pH 9.0 the Cys —SH of KLC is predominantly a thiolate anion (pKₐ 8-8.5 depending upon the molecular environment of the sulfhydryl cysteine). The introduction of the anionic charge facilitates a change in the interaction of the peptide with the MPO active site. The interactions consist of i) an N-terminus amino acid #1 (AA₁), with an amine functional group (e.g. Lysine (K)) and the Glutamate 268 of the MPO active site. ii) Amino acid #2 (AA₂), contains a hydrophobic amino acid side-chain (Leucine (L)) that cannot stabilize a radical species, but can lock with the heme group via a series of hydrophobic interactions iii) Amino acid #3 (AA₃,) contains a heteroatom (the sulfhydryl sulfur of Cys), in the form of a thiolate anion, that can induce a conformational change due to a ionic interaction with the MPO active site. In addition, the Cys -Sulfur can ultimately bear the free radical species generated by MPO active site complex.

v) Different MOA-KVC and KVVC Peptides

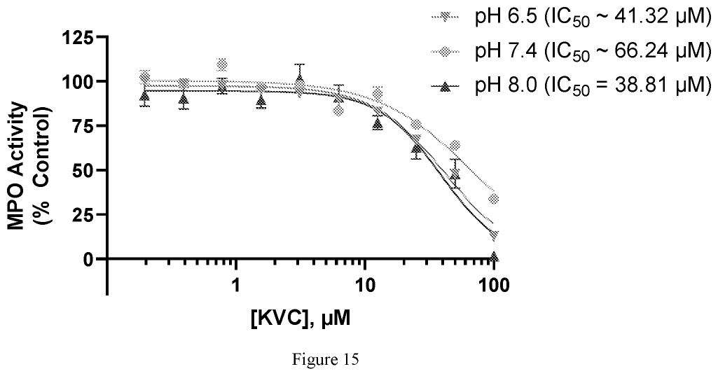

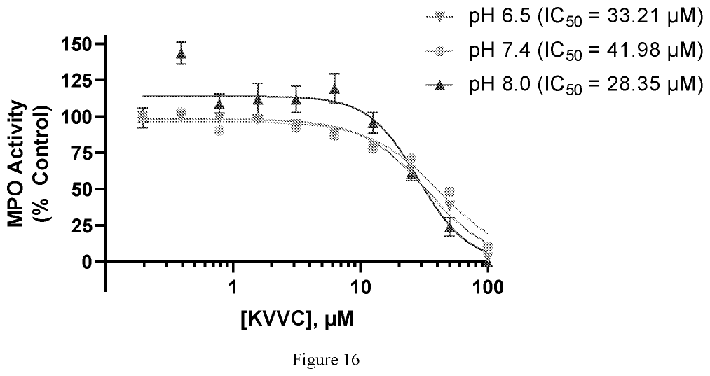

In this case the addition of an amino acid, KVC versus KVVC results in an improvement of inhibitory properties (i.e. IC₅₀ numerical values decrease—see FIG. 16) of the pH regardless of pH. See FIGS. 15 and 16, respectively. This is a result of enhanced hydrophobic interactions for KVVC with the heme of MPO active site, and possible enhanced proximity of the cys —SH group to the iron/heme complex I.

Taken together, the data disclosed herein suggests that the structural features of the AA₍ₙ₎ peptides (where n can be 2-5) are important for determining the effectiveness of a library of model peptides that can inhibit MPO toxic oxidant production. Data in this disclosure describe the structural and biochemical features of peptide inhibitors of MPO toxic oxidant production. Findings presented in the current disclosure show that peptides must possess a number of distinct physicochemical properties to inhibit MPO toxic oxidant production. In addition depending on the sequence and amino acid composition inhibition may occur through different MOA pathways. Also these rules are applicable in the design of a library of organic molecules.
Data shown here for the first time demonstrate that the AA₍ₙ₎ peptide model of MPO inhibitors identifies new mechanisms not previously described. Accordingly, the AA₍ₙ₎ peptide model KXZ expands the number of amino acids that can be used to make peptide inhibitors of MPO toxic oxidant production beyond any previous teachings.

### Example 3

### Demonstrating Systems Chemico-pharmacology Properties for Exemplary KXZ Tripeptide in Inhibiting Pro-inflammatory Peptides and Proteins

KYZ is reduced (gains an electron in the form of a radical) in the presence of MPO, which subsequently decreases the production of hypochlorous acid. All MPO activities (i.e., hypochlorous acid production) were quantified by the 3,3′,5,5′-tetramethylbenzidine (TMB) assay. The tripeptide KXZ, requires amino acid #1 (AA₁, K) to be a native amino acid or an artificial amino acid (aa) with a basic side-chain such as an amine functional group (e.g., Lysine (K)). Amino acid #2 (AA₂, X) is a native or artificial amino acid that may contain a polar, non-polar, or aromatic amino acid. Amino acid #3 (AA₃, Z) is a native or artificial amino acid that possesses a heteroatom that can stabilize a free radical that then goes on to form a heteroatom-carbon bond in the form of a homo- or heterodimer of the tripeptide.

Although no one mode of operation is adopted herein, it is postulated that when KXZ enters the active site of MPO (and presumably any other Peroxidase), it is “activated” through a series of redox chemistry processes and KXZ* is created (where Z* is a radical located on the heteroatom (HA) [e.g. S, or Se or other HAs] of the Z amino acid side-chain). The radical of KXZ* must be located on a HA that can form a new “lower energy” chemical bond such as —S—R. In addition, the KXZ* may go on to form either a neutral homodimer (KXZ)₂, radical homodimer (KXZ)₂ *, or a radical heterodimer (KXZ-GSH)*. This process can play a role in facilitating the regeneration of neutral KXZ via the GSH pathway.

However, a second process occurs, and is postulated to occur as follows although no one mode of operation is adopted herein: (1) KXZ* is formed in the active site of MPO; (2) KXZ* exits the active site and interacts with GSH to form (KXZ-GSH)* (where R is a molecule of neutral GSH), which is relatively more stable than KXZ* alone; and (3) (KXZ-GSH)* then can react with a PROXIMAL peptide/protein containing an accessible, free —SH group (assuming, but not restricted to, from Cys or homocysteine), which results in the thiolation of the peptide/protein to form P-KXZ (where P represents the peptide or protein).

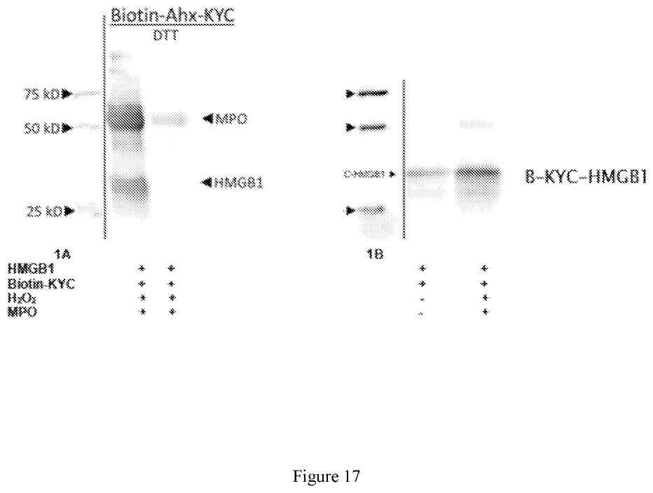

In support of this activity, FIG. 17 illustrates MPO-dependent KXZ thiolation of the protein HMGB1. Our data show that MPO oxidation of KXZ results in KXZ thiolation of HMGB1. The thiolation of HMGB1 results in the loss of this protein activity, a known proinflammatory entity, resulting in a marked reduction in inflammation when KXZ and MPO are present. Said alternatively, KXZ exploits MPO to generate a new agent that reduces inflammation by thiolating HMGB1 and preventing its ability to recruit myeloid cells and increase oxidative stress and TLR4- and RAGE-dependent inflammation.

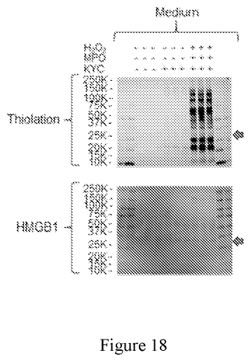

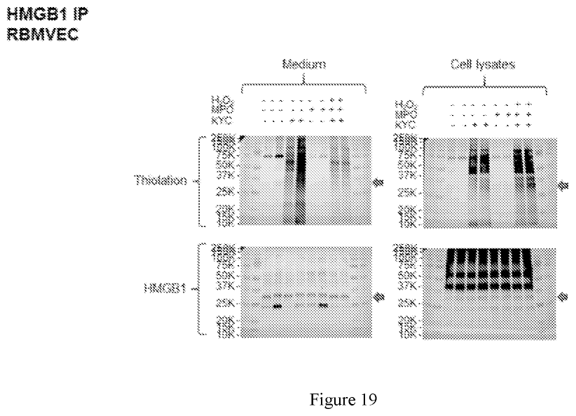

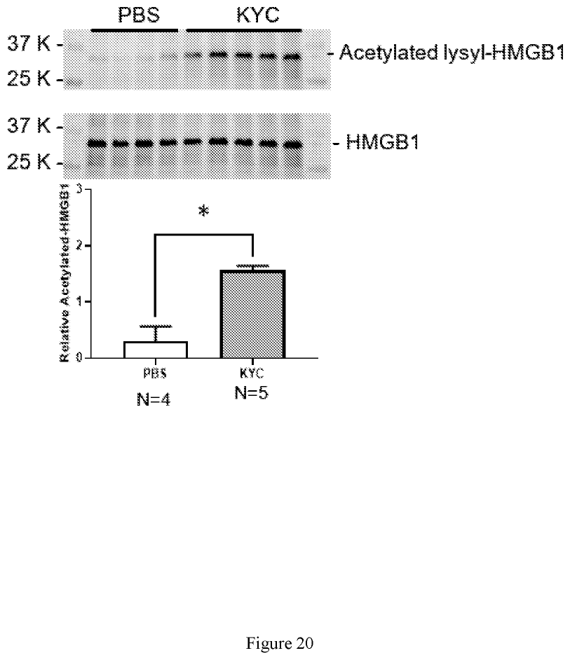

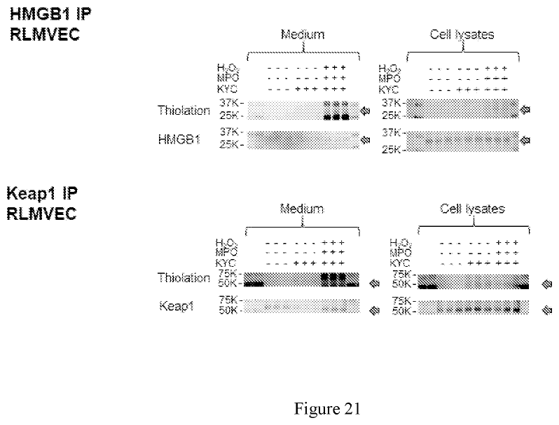

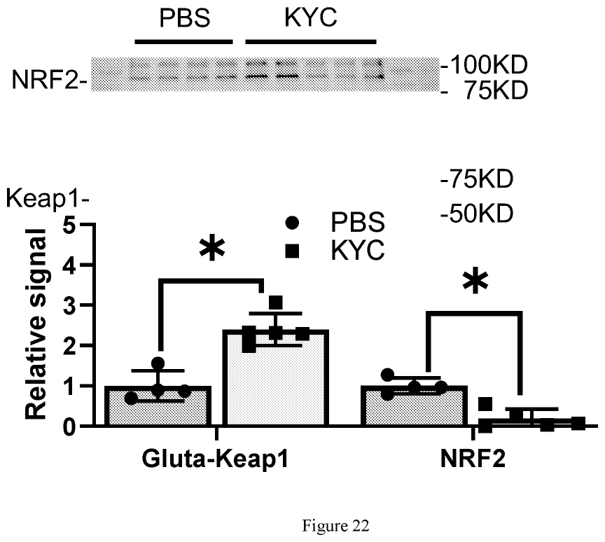

The incubation of Endothelial cells in the presence of MPO and H₂O₂ with KYC results in the production of KYC* (* represents radical). The resultant activated peptide KYC* can then go on further to modify specific proximal proteins such as HMGB1. The HMGB1-thiolated protein with KYC no longer functions in a pro-inflammatory manner. As a consequence downstream proteins, such as Nrf2 are also significantly ameliorated in their pro-inflammatory actions (see FIGS. 18-22).

### Example 4

### N-Acetyl-Lysyltyrosylcysteine Amide, a Novel Systems Pharmacology Agent, Reduces Bronchopulmonary Dysplasia in Hyperoxic Neonatal Rat Pups

Abstract

Bronchopulmonary dysplasia (BPD) is caused primarily by oxidative stress and inflammation. To induce BPD, neonatal rat pups were raised in hyperoxic (>90% O2) environments from day one (P1) until day ten (P10) and treated with N-acetyl-lysyltyrosylcysteine amide (KYC). In vivo studies showed that KYC improved lung complexity, reduced myeloperoxidase (MPO) positive (+) myeloid cell counts, MPO protein, chlorotyrosine formation, increased endothelial cell CD31 expression, decreased 8-OH-dG and COX1/COX2, HMGB1, RAGE, TLR4, had little effect on weight gain but improved survival in hyperoxic pups. EPR studies showed that MPO reaction mixtures oxidize KYC to a KYC thiyl radical. Adding recombinant HMGB1 to an MPO reaction mixture containing KYC resulted in KYC thiylation of HMGB1. In rat lung microvascular endothelial cell (RLMVEC) cultures, KYC thiylation of RLMVEC proteins was increased the most in RLMVEC cultures treated with MPO+H2O2, followed by H2O2, and then KYC alone. KYC treatment of hyperoxic pups decreased total HMGB1 in lung lysates, increased KYC thiylation of HMGB1, terminal HMGB1 thiol oxidation, decreased HMGB1 association with TLR4 and RAGE, and shifted HMGB1 in lung lysates from a non-acetylated to a lysyl-acetylated isoform, suggesting that KYC reduced lung cell death and that recruited immune cells had become the primary cellular sources of HMGB1 released in the lung. MPO-dependent and independent KYC-thiylation of Keap1 were both increased in RLMVEC cultures. Treating hyperoxic pups with KYC increased KYC thiylation and S-glutathionylation of Keap1, Nrf2 activation. These data, taken together, suggest that KYC is a novel system pharmacological agent that exploits MPO to inhibit toxic oxidant production and to be oxidized into a thiyl radical that inactivates HMGB1, activates Nrf2, and increases antioxidant enzyme expression to improve lung complexity and reduce BPD in the lungs of hyperoxic rat pups.

Introduction

Bronchopulmonary dysplasia (BPD) affects more than 10,000 infants annually [1], making it the most common pulmonary morbidity of premature infants in the United States [2]. BPD is caused by complications from respiratory distress syndrome (RDS) of the newborn. Currently, treatments for RDS include surfactant, caffeine, supplemental oxygen, and gentle mechanical ventilation [2]. Although supplemental oxygen is essential for preventing hypoxemic organ injury in premature neonates, supplemental oxygen always increases oxidative stress and inflammation. One way hyperoxia increases inflammation is by activating resident myeloid cells and vascular endothelial cell NADPH oxidase (NOX) dependent superoxide anion production [3-5]. Activated resident myeloid cells release MPO and also generate superoxide anion, which dismutates into hydrogen peroxide (H2O2) to provide the substrate for MPO to oxidize chloride or nitrite anions into hypochlorous acid (HOCl) [6] or nitrogen dioxide radical (NO2*) [7], respectively.

A consequence of chronic increases in oxidative stress and inflammation in the lung is pulmonary cell injury and cell death. Dead and dying cells release high mobility group box1 (HMGB1). Under normal conditions, HMGB1 serves as a nuclear DNA binding protein that performs many vital roles in maintaining DNA structure and regulating gene expression [8]. However, after cells die or immune cells and platelets become activated, HMGB1 is released into extracellular spaces, where it turns into a danger-associated molecular pattern (DAMP) molecule that possesses potent cytokine- and chemokine-like properties [9, 10]. HMGB1's ability to recruit myeloid cells to the lung begins to explain how hyperoxia and excessive mechanical ventilation increase myeloid cell recruitment, and MPO-dependent oxidative damage to the premature neonate's lung to induce a form of lung injury [11] that is far greater than that which is initially induced by hyperoxia. Support for HMGB1 playing a causal role in inflammation in BPD comes from clinical studies showing that the concentration of HMGB1 in tracheal aspirates directly correlates with BPD severity and death in ventilated premature neonates [12].

One of the reasons premature neonates are at increased risk of BPD is that they often lack the antioxidant enzymes that protect against the oxidative stress induced by supplemental oxygen [13, 14]. Exactly why premature neonates lack antioxidant enzymes is unclear. A potential explanation however is that hypoxic in utero environments do not require a robust antioxidant system and some premature neonates do not respond to hyperoxia appropriately, possibly because of genetic predispositions [14]. However, reports by Cho and Kleeberger [15-17] have defined the relationship between Keap1 and Nrf2, linked hyperoxic lung injury to Nrf2 [18], and confirmed that Nrf2 is the primary regulatory pathway for modulating antioxidant enzyme expression in the lung [19]. Although developing antioxidant agents that scavenge multiple oxidants is attractive [14], effective targeting of oxidative stress to reduce lung injury in neonates has not been achieved and optimizing scavengers to target specific oxidants and free radicals is extremely difficult. As hyperoxia has been linked to Nrf2 [18], and premature neonates who are at risk of hyperoxic lung injury lack the antioxidant enzymes required for protection [13, 14], it might be possible to exploit the Keap1/Nrf2 signaling pathway to protect premature neonates against supplemental oxygen.

The above reports, taken together, suggest that BPD is a complex, multifactorial disease process that is induced primarily by oxidative stress and inflammation. Such a statement implies that a systems biology approach would be useful for treating BPD. However, to our knowledge, no such agent exists. If a therapeutic agent could be developed to inhibit MPO, inactivate HMGB1, or active Nrf2, then such an agent might be useful for decreasing oxidative stress and inflammation in the lungs of neonates that lung development will proceed even in high-risk premature neonates treated with supplemental oxygen.

In the present study, we report on the effects of KYC on BPD in hyperoxic neonatal rat pups. KYC is a tripeptide that was initially designed to inhibit MPO toxic oxidant (HOCl) production and increase MPO-dependent catalase activity [20]. Our studies suggest that KYC exploits MPO peroxidase activity to be oxidized into a KYC thiyl radical that inactivates HMGB1 and activates Nrf2. When hyperoxic neonatal rat pups are treated with KYC, oxidative stress and inflammation decrease, and antioxidant enzymes increase. Studies here show that KYC prevents oxidative lung injury and improves lung development in neonatal rat pups raised in hyperoxic environments.

Material and Methods

Peptide Synthesis: KYC and biotin-aminohexanoyl-N-[Ahx]-KYC-amide or biotinylated-KYC (B-KYC) were synthesized using Fmoc [N-(9-fluorenyl)methoxy-carbonyl] chemistry, prepared and purified as an acetate salt by Biomatik USA, LLC (Wilmington, Del.) as previously described [21-23]. Trifluoroacetic acid (TFA) in the tripeptide preparations was removed by dissolving KYC in distilled water containing 6 mM HCl followed by lyophilization (2-3×). TFA in KYC and B-KYC was quantified by NMR in the Biomolecular NMR Facility at MCW [24]. TFA was reduced to <0.01% before being used for experiments.

Rats and Experimental Protocols: Time-dated pregnant Sprague-Dawley rats were obtained from Envigo (Madison, Wis.) and acclimated in the animal facility for one week. Animal protocols were approved by MCW's Institutional Animal Care and Use Committee and conformed to NIH Guide for the Care and Use of Laboratory Animals. Animals were housed in barrier cages with a 12-h dark-light cycle and were given free access to chow and water. Pups from two dams were mixed and randomly distributed to each nursing dam by balancing sex and size of the pups. The sex of the pups was determined from the relative distance between the genitalia and anus. The dam and pups were placed in cages in either room air (normoxia) or in >90% oxygen (hyperoxia) in an enclosed chamber from postnatal day 1 to day 10 (P1-P10) to induce BPD as previously reported [25, 26]. Oxygen concentrations were continuously monitored with an oxygen sensor (Reming Bioinstruments Co., Redfield, N.Y.).

Since the number of neonatal rat pups per pregnant dam limits the number of experimental conditions that can be compared, we modified our standard experimental protocol. The first study was designed to determine if hyperoxia increased BPD and caused a change in proteins of interest. Data from these studies can be found in on-line supplemental data. The second study was designed to determine if inhibiting MPO toxic oxidant production reduced BPD. At least three sets of neonatal pups from three different dams were used for each study. Pups were fed ad libitum from nursing dams. Pups were inspected daily starting on P1 and weighed starting on P3 in room air for less than ten minutes. Nursing dams were switched daily to avoid the impact of oxygen toxicity. Experience has taught us that severity of hyperoxia-induced BPD is litter dependent. To minimize litter differences in both types of experiments pups from different litters were mixed and randomly reallocated to the nursing dams.

Previously we reported that KYC was highly effective at reducing MPO-dependent oxidative injury to cultured endothelial cells and that KYC had little to no effect on endothelial cell viability, apoptosis, necrosis, or mitochondrial function when added to culture media up to 4000 μM [27]. In other studies, we observed that injection of increasing single doses (IP) of KYC into control mice did not induce observable adverse effects until the dose reached 800 mg/kg (unpublished, personal observations). KYC has been used in a variety of animal models and has been shown to reduce chemically-induced tumor formation [28], vasculopathy in sickle cell mice [21], secondary brain injury in middle cerebral artery occlusion mice [22, 29], neurological disease scores in EAE [23, 30], and improve vasculogenesis and reduce neutrophil infiltration in hindlimb ischemia in diabetic mice [31]. With such an abundance of murine models of vascular disease and injury showing that KYC effectively reduced myeloid cell recruitment and oxidative damage, with little to no evidence of toxicity, we focused our efforts on determining if KYC reduced BPD in hyperoxic neonatal rat pups.

No KYC dose-response studies were performed in neonatal rat pups in preparation for the current study. Instead, we decided to treat neonatal rat pups empirically with KYC at 5 mg/kg twice per day based on published and unpublished data in adult mice. A review of doses in previous studies in adult mice suggests that the therapeutic window for KYC is from 0.3 mg/kg (once per day) [23] and up to 15 mg/kg (twice per day) [30]. The higher dose is slightly more than 26 times less than the concentration of KYC that was observed to induce adverse effects. The dose of KYC used in the current study is based on our experience in treating chronic inflammation in other disease states [21, 23, 30] and is within the range that we consider appropriate for neonatal rat pups and adult rats.

To determine the effects of KYC on BPD in hyperoxic neonatal rat pups we randomly assigned pups to the PBS injection control group, or the KYC treatment group. KYC was injected (intraperitoneally, IP), starting at P2 to half of the randomly assigned pups in each litter using a sterile insulin syringe fitted with a 30G needle (Beckon Dickinson, New York, N.Y.) until P10. To determine the effects of KYC on KYC thiylation of HMGB1 and Keap-1, pups were injected IP twice daily with KYC at P2-P9, and then once with B-KYC (5 mg/kg), or an equal volume of phosphate buffer solution (PBS) . Pups were euthanized at P10 with carbon dioxide and lungs removed en bloc. A small cut was created on the left atrium and ice-cold normal saline was gently infused through the right ventricle to flush blood from the lungs before inflation for histology or snap-frozen in liquid nitrogen for protein studies. For weight-gain and survival studies, comparisons were made between the effects of normoxia and hyperoxia and between the effects of KYC and PBS on hyperoxic pups. Weight was recorded starting on P3 and death was recorded each day from P1 to P10.

Cells, Materials, and Antibodies:

Rat lung microvascular endothelial cells (RLMVEC) from neonatal Sprague-Dawley rats (RN-6011) and rat endothelial growth medium (EGM, M1266SF) were from Cell Biologics (Chicago, Ill.). Amicon®Ultra-4 centrifugal concentrators with 3K cut-off (UFC800324) were from Merck Millipore Ltd (Carrigtwohill, Ireland). PierceTM Streptavidin (#88816), Protein G (#88848), Protein A Magnetic Beads (#88845), PierceTM ECL Western Blotting Substrate (#32106), and SuperSignalTM West Femto Maximum Sensitivity Substrate (#34095) were from Thermo Fisher Scientific (Waltham, MA). Rabbit antibodies for myeloperoxidase heavy chain (MPO, sc-33596) and TLR4 (sc-10741) were from Santa Cruz Biotechnology (Dallas, Texas). Rabbit antibody for Cl-Tyr (HP5002) was from Hycult Biotech (Plymouth Meeting, PA). The chicken antibody for HMGB1 (326052233) was from SHINO-TEST Corporation (Kanagawa, Japan). Rabbit antibody for RAGE (GTX23611) was from GeneTex (Irvine, CA). Mouse antibodies for PECAM-1/CD31 (ab24590), for 8-OH-dG (ab48508), and for rat endothelial cell antigen-1 (RECA-1, ab9774) were from Abcam (Cambridge, MA). Rabbit antibody for Cox1 (sc-7950) and Cox2 (sc-7951) were from Santa Cruz Biotechnology. Mouse (ab190377) and rabbit (ab77302b) antibodies for HMGB1, and streptavidin-HRP (ab7403) were from Abcam (Cambridge, Mass.). Rabbit antibody for acetylated-HMGB1 (OASG03545) was from Aviva System Biology (San Diego, Calif.). Rabbit antibodies for Nrf2 (sc-722) and TLR4 (sc-30002) were from Santa Cruz Biotechnology (Dallas, Tex.). Rabbit antibody against Keap1 (ABS97) and mouse antibody for β-actin (A2228) were from Millipore-Sigma (St. Louis, Mo.). Rabbit antibody for cysteine sulfonyl (ADI-OSA-820) was from Enzo Life Sciences (Farmingdale, N.Y.). Mouse antibody for glutathionylated proteins (D8) and all other chemicals were from Sigma-Aldrich (St. Louis, Mo.). All antibodies were certified either by the manufacturer or were used previously and certified by the authors, as was the case for the anti-HMGB1 antibody [32].

Immunohistochemistry and Immunocytochemistry:

After euthanasia, the trachea was cannulated with an Instech Solomon (20G) stainless steel feeding tube (Plymouth Meeting, Pa.) and the lungs were inflated with 10% neutral buffered formalin at 25 cm-H2O (2.4 kPa) for one hour. After the trachea was securely tied to the stainless feeding tube to maintain pressure with surgical silk, the lungs were perfusion fixed with an additional aliquot of 10% neutral buffered formalin, surgically removed and then stored in 10% buffered formalin for 24 h before paraffin embedding. Lung sections (5 μm) were mounted on SuperFrost plus-coated slides (Denville Scientific, Metuchen, N.J.). Slides were deparaffinized, and sections stained with hematoxylin and eosin (H&E). Histology images were captured with a mounted digital camera using an Olympus IX 51 microscope and a 10× objective. Inflammatory cells, myeloid cells, including neutrophils, monocytes, and macrophages, were stained with MPO antibody (1:200) overnight at 4° C. and HPR-conjugated anti-rabbit antibody (1:1000) at room temperature for one hour then visualized by diaminobenzidine to generate a dark-brown color. Blood vessels were stained with RECA-1 and visualized with horseradish-conjugated secondary antibody and diaminobenzidine. Immunofluorescence of 8-OH-dG was used as a biomarker for oxidative DNA damage. Lung sections were stained with the 8-OH-dG antibody (1:100) for overnight at 4° C. then treated with AlexaFluor488-conjugated secondary antibody for one hour in room temperature and counterstained with DAPI before imaging with a fluorescent microscope. The average of three sections per pup, and five counts per section (15 counts/pup) was used for statistical analysis. Quantified data were obtained and entered into the record using predetermined codes by one of the coauthors who was not involved in taking the images.

Morphometric Analysis:

The mean linear intercept (MLI), or chord length, was used as a method to estimate the volume-to-surface ratio of acinar airspaces whereas radial alveolar count (RAC) and secondary septa were investigated to study the complexity of lung structure [33]. Ten equally spaced horizontal lines were drawn on each picture, and the number of intercepts through the alveolar wall was counted. MLI was obtained by multiplying the number of times the traverses are placed on the lung times the length of the traverse and dividing the result by the sum of all the intercepts. For RAC, a line from the center of the respiratory tract perpendicular to the nearest connective tissue septum was drawn, and alveoli intercepting with the line were counted. For measurement of secondary septa, elastin was stained with resorcin fuchsin and Van Gieson's solution.

Immunoblot Analysis: Whole lung lysates were obtained by homogenizing in MOPS buffer (20 mM 3-N-morpholino-propane sulfonic acid, 2 mM EGTA, 5 mM EDTA, 30 mM NaF, 10 mM β-glycerophosphate, 10 mM Na pyrophosphate, 2 mM Na orthovanadate, 1 mM PMSF, 0.5% NP-40, 1% protease inhibitor cocktail, and 1% phosphatase inhibitor cocktails 2 and 3, pH 7.0) by Bullet Blender (Next Advance, Inc., Averill Park, N.Y.). For protein immunoblots, 30 μg of protein lysate was separated by SDS-PAGE, transferred to nitrocellulose membranes (0.22 μm), and then probed with the appropriate primary antibodies overnight at 4° C. on a continuously rocking platform. Signals were generated after incubation with horseradish peroxidase-conjugated goat anti-rabbit or anti-mouse IgG (1:8,000) using Pierce™ ECL Western Blotting Substrate or SuperSignal™ West Femto Maximum Sensitivity Substrate. Integrated optical density (IOD) was calculated using ImageJ software and normalized to β-actin, a loading control.

Statistical Analysis:

Representative images of lung sections and immunoblots are presented in the figures. Data from lung sections and immunoblots from the samples were analyzed for statistical differences using GraphPad Prism version 8.4.3, for Windows (GraphPad Software, La Jolla, Calif.). Quantitative morphometric and cell count data are expressed as mean±SD scatter-plots adjacent to the representative images. Student t-test, or Mann-Whitney U test, was used for comparing two groups wherever appropriate. One-way analysis of variance with post-hoc Student-Newman-Keuls test was used when more than two groups of data were analyzed. Significant differences between groups were determined by either unpaired student's t-test or Mann-Whitney U test, depending on the distribution. Weight gain and survival data were analyzed using MedCalc Statistical Software version 15.2.1 (MedCalc Software bvba, Ostend, Belgium; http://www.medcalc.org). Kaplan-Meier survival curves were plotted using Kaplan-Meier tables constructed in GraphPad Prism. A p-value<0.05 was considered statistically significant.

Results

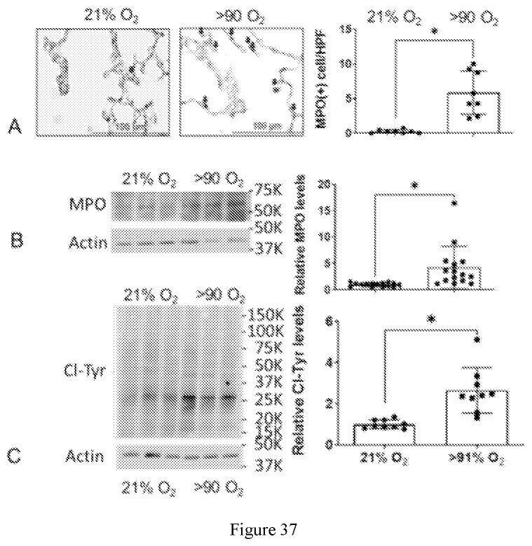

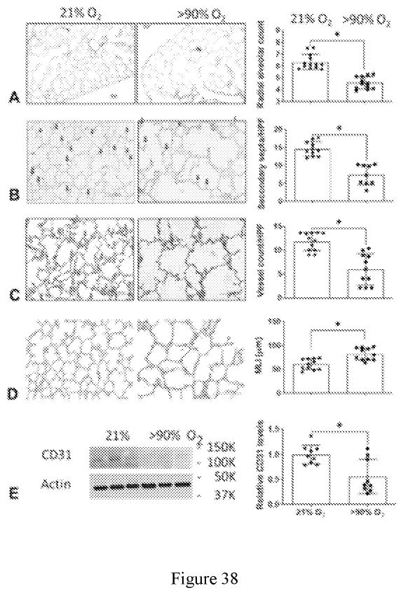

Effects of Hyperoxia on BPD in Neonatal Rat Pups: Chronic hyperoxia increased BPD in neonatal rat pups on P10 based on differences in lung morphometrics and various mechanism-based biomarkers of oxidative stress and inflammation (see supplemental FIGS. 37 and 38).

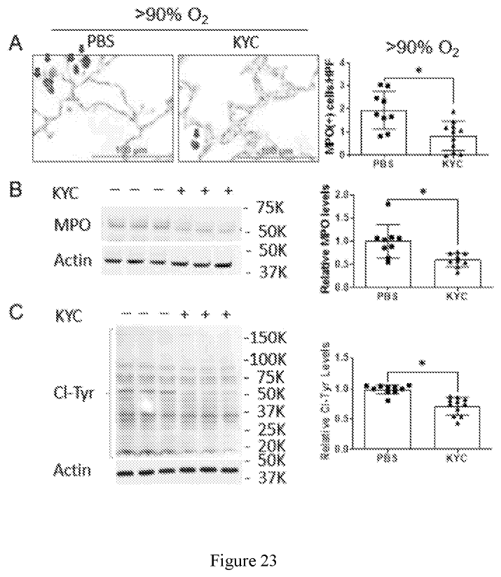

Effects of KYC on Hyperoxia-induced Myeloid Cell Recruitment, MPO, and Oxidative Stress: KYC reduced the number of MPO+ myeloid cells in hyperoxic lungs by approximately 50% (brown cells in FIG. 23A). MPO immunoblots show that KYC reduced MPO protein by approximately 50% (FIG. 23B) and Cl-Tyr formation by approximately 27% (FIG. 23C).

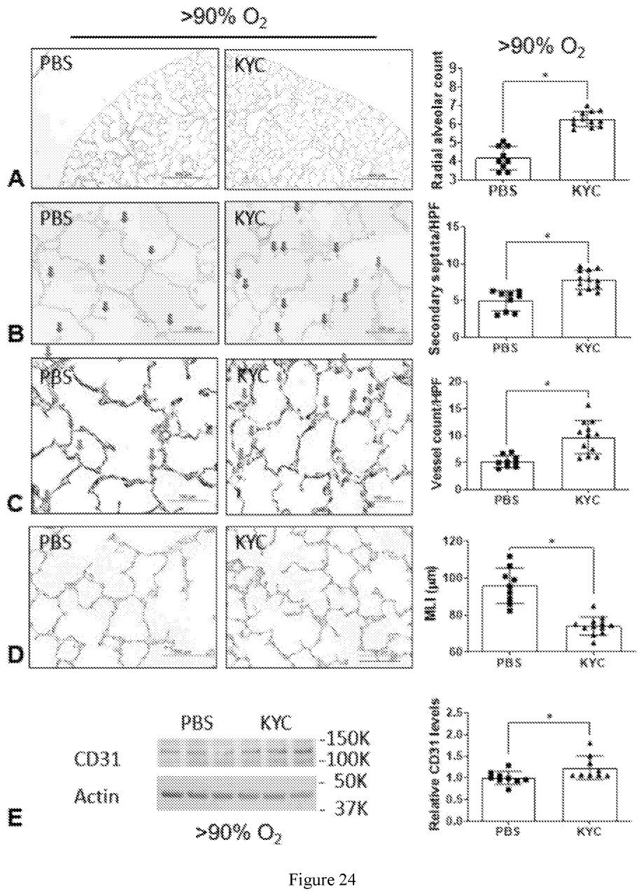

Effects of KYC on Hyperoxia-induced BPD: If MPO plays a causal role in the mechanisms by which hyperoxia induces BPD, then KYC, which inhibits MPO toxic oxidant production but not peroxidase activity [27], should reduce hyperoxia-induced changes in lung architecture. KYC treatment of hyperoxic pups increased RAC, secondary septa, and blood vessel counts by approximately 35%, 55%, and 92%, respectively, while decreasing MLI by approximately 22% (FIG. 24A-2D). Immunoblots for CD31 confirm that KYC increased the number of endothelial cells in hyperoxic lungs (approximately 23% more) (FIG. 24E). The increase in CD31 expression supports immunohistochemistry data showing that KYC increased microvascular blood vessels and alveolar complexity that are both essential for improving lung architecture and decreasing BPD in hyperoxic pups.

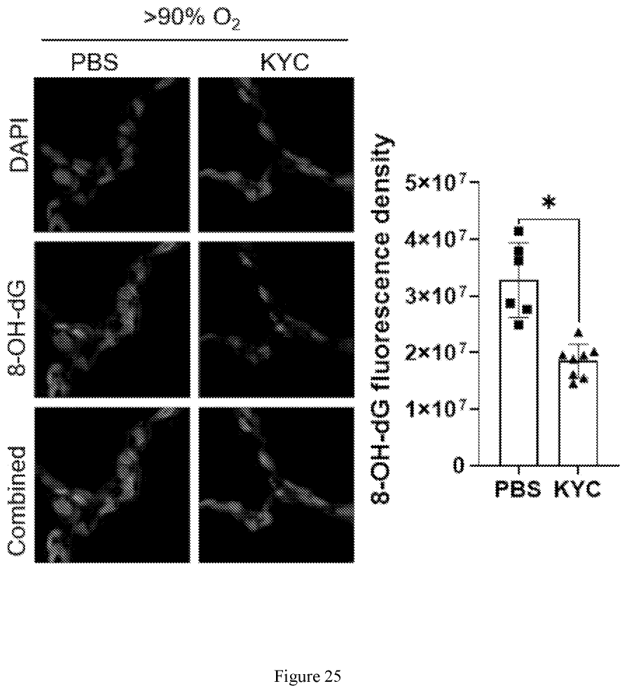

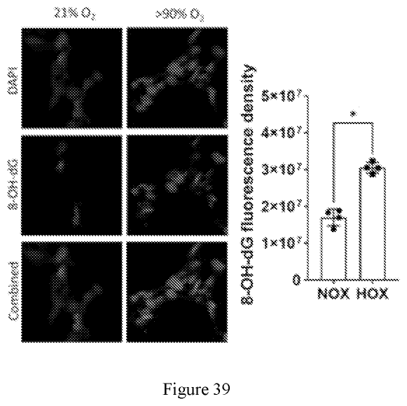

Effects of KYC on 8-OH-dG in Hyperoxic Lungs: Hyperoxia increased 8-OH-dG, a biomarker of DNA oxidative damage in the lungs of neonatal rat pups (see supplemental FIG. 39). Immunofluorescence staining for 8-OH-dG showed that KYC treatment reduced 8-OH-dG in the nuclei of lung cells in hyperoxic rat pups (FIG. 25).

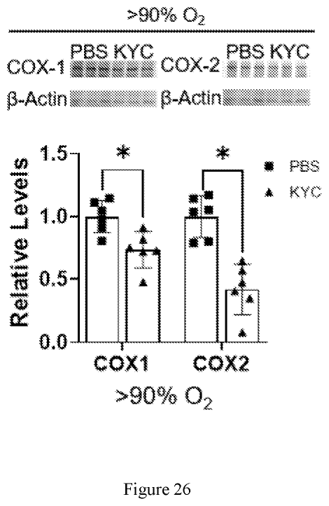

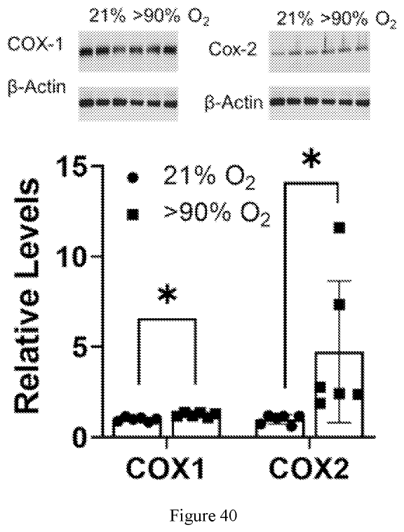

Effects of KYC on Cox1/Cox2 in Hyperoxic Lungs: Hyperoxia increased Cox1 slightly and Cox2 markedly in the lungs of neonatal rat pups (see supplemental FIG. 40). KYC treatment of hyperoxic neonatal rat pups reduced pulmonary Cox1 by 20-30% and Cox2 by about 60-70% (FIG. 26).

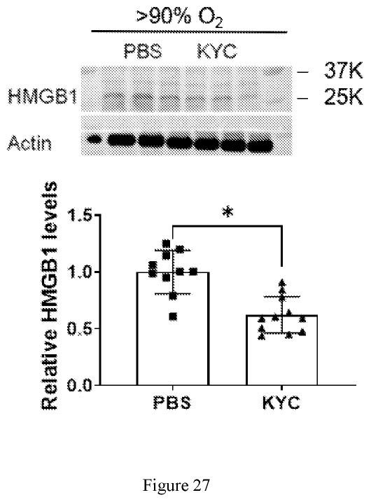

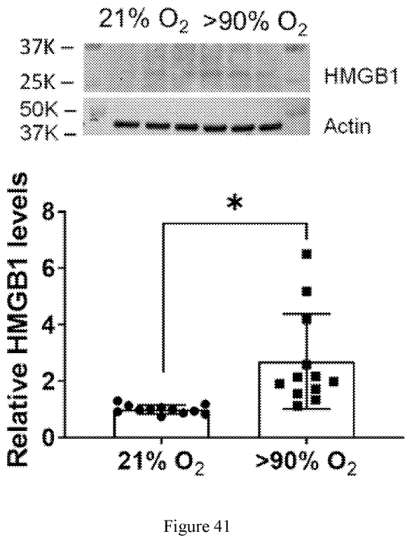

Effects of KYC on HMGB1 in Hyperoxic Lungs: Hyperoxia increased HMGB1 levels in lung lysates of neonatal rat pups (see supplemental FIG. 41). KYC treatment of hyperoxia pups decreased HMGB1 levels in lung lysates from hyperoxic pups by approximately 38% (FIG. 27).

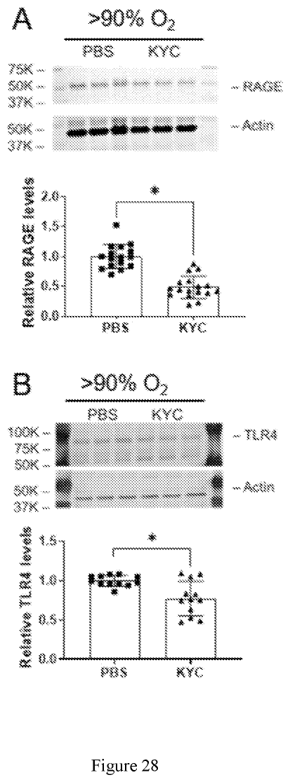

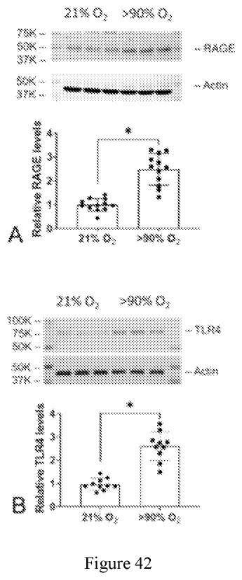

Effects of KYC on RAGE and TLR4 in Hyperoxic Lungs: Hyperoxia increased RAGE and TLR4 in lungs of neonatal rat pups (see supplemental FIG. 42). KYC treatment reduced the expression of RAGE and TLR4 in lung lysates from hyperoxic pups by 60% and 22%, respectively (FIGS. 28A and 28B).

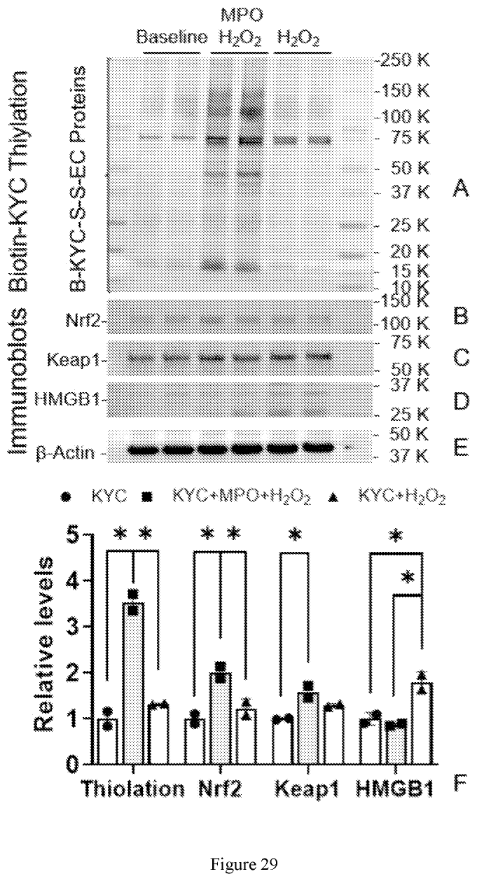

Effects of Oxidative Stress on KYC Thiylation on Endothelial Cell Proteins in RLMVEC Cultures: To determine the effects of oxidative stress on KYC thiylation, RLMVEC cultures were treated with B-KYC to follow KYC thiylation with streptavidin-affinity blotting as outlined in Methods. Three different levels of oxidative stress were used to determine the effects of oxidative stress on KYC thiylation of endothelial cell proteins: media alone (baseline); media containing MPO+H₂O₂ (MPO-dependent); and media containing H₂O₂ alone (H₂O₂-dependent) (FIG. 29A). KYC thiylation of RLMVEC proteins at baseline was low (FIG. 29A, first two lanes). MPO-dependent KYC thiylation of RLMVEC proteins was markedly increased (FIG. 29A, middle two lanes). H₂O₂-dependent KYC thiylation of RLMVEC proteins was more than observed at baseline, but not as high as MPO-dependent B-KYC thiylation. These studies show that MPO-dependent thiylation induces the greatest level of KYC thiylated RLMVEC proteins (FIG. 29F, first three bars).

Immunoblot band densities for Nrf2 (29B), Keap1 (29C), and HMGB1 (29D) relative to β-actin (29E) reveal that protein expression for the three proteins is differentially modulated by oxidative stress. Nrf2 expression is increased in RLMVEC cultures subjected to MPO-dependent KYC thiylation more than in RLMVEC cultures subjected to KYC thiylation under baseline or H₂O₂-dependent oxidation (FIG. 29F, second three bars). Keap1 expression followed a pattern similar to Nrf2 (FIG. 29F, third 3 bars). In contrast, HMGB1 expression increased in RLMVEC cultures subjected to H₂O₂-dependent KYC thiylation more than in RLMVEC cultures subjected to KYC thiylation at baseline or in RLMVEC cultures subjected to MPO-dependent KYC thiylation (FIG. 29F, the fourth set of 3 bars). These data are consistent with the idea that MPO-dependent KYC thiylation is protective, while H₂O₂-dependent KYC thiylation may be injurious even when KYC is present because MPO isn't available to degrade H₂O₂ [27], which would reduce oxidative stress.

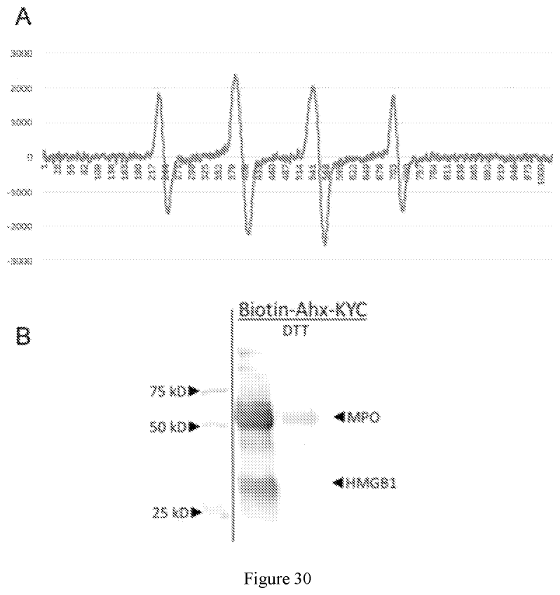

MPO Oxidizes KYC to a Thiyl Radical that Thiylates HMGB1: FIG. 30A shows a four-line ESR spectrum corresponding to DMPO-SO-KYC. This spectrum is similar to the spectrum of DMPO-S◯-YC generated when the dipeptide YC was added to an MPO reaction mixture containing DMPO [34]. FIG. 30B shows an autoradiogram of the bands corresponding to MPO and HMGB1 from a fluorescent streptavidin-affinity blot. The fluorescent density of the bands for B-KYC thiylated HMGB1 and MPO are decreased in the sample treated with dithiothreitol (DTT (100 mM, final concentration, second lane). As DTT is a potent disulfide reducing agent, the decrease in band density confirms that the bonds between KYC and HMGB1 and MPO are disulfides.

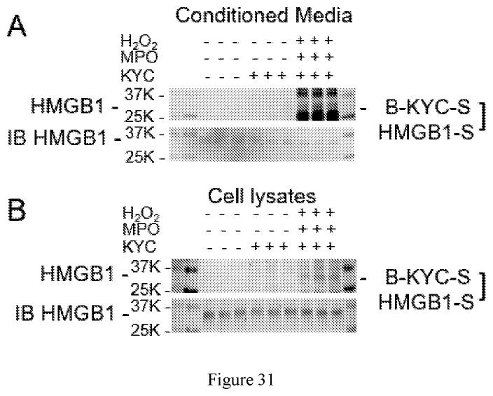

MPO Oxidation of KYC Results in KYC Thiylation of HMGB1 Released from RLMVEC Cultures: To determine if MPO oxidizes KYC to a thiyl radical that thiylates HMGB1 released from RLMVEC cultures, proteins in the media and the cell lysates from RLMVEC cultures at baseline with no KYC, with KYC and with MPO+H₂O₂+KYC were affinity blotted for KYC-thiylated proteins and immunoblotted for HMGB1. HMGB1 immunoblots showed that all RLMVEC cultures released low amounts of HMGB1 (FIG. 31A, lower blot, all nine lanes). However, KYC thiylation of HMGB1 was greater in RLMVEC cultures incubated with MPO+H₂O₂+KYC (FIG. 31A, upper blot, last three lanes) than the other two conditions. Similar differences across the three test groups can be seen in the streptavidin affinity blots of cell lysates (FIG. 31B, upper blot) when compared in the context of the immunoblots for HMGB1 (FIG. 31B, lower blot) with the one caveat that the extracellular HMGB1 in FIG. 31A was KYC thiylated to a much greater extent than the intracellular HMGB1 in RLMVEC lysates in FIG. 31B. This conclusion is based on differences in band densities for KYC-thiylated HMGB1 in the upper blot as a function of HMGB1 protein in the lower blot. These data are consistent with the idea that MPO oxidizes KYC to a thiyl radical that thiylates HMGB1 released from RLMVEC cultures.

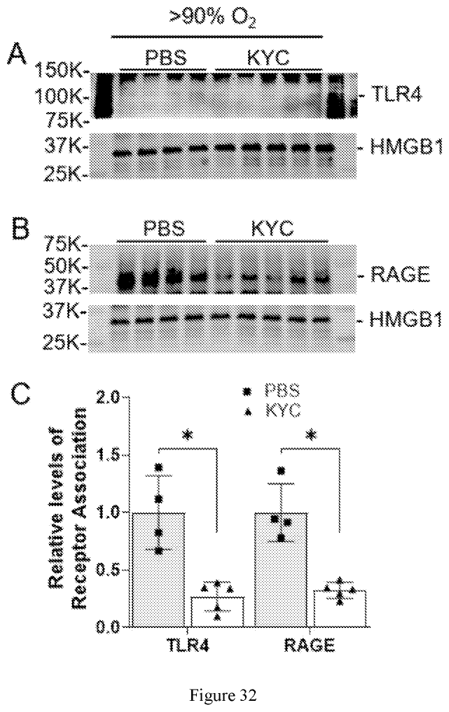

Effects of KYC Treatment on HMGB1 Association with TLR4 and RAGE in Hyperoxic Lungs: Although KYC treatment reduced total HMGB1 in lung lysates of hyperoxic neonatal rat pups (FIG. 27), precisely how KYC thiylation of HMGB1 alters the interaction of HMGB1 with TLR4 and RAGE is unknown. HMGB1 pulldown assays revealed that KYC treatment decreased HMGB1 association with TLR4 in lung lysates from hyperoxic neonatal rat pups (FIGS. 32A and 32C). Please note, even though the binding affinity of HMGB1 for TLR4 is reported to be weak [35], the band corresponding to HMGB1 was still visible in all lanes (FIG. 32A). KYC treatment also reduced HMGB1's association with RAGE in the lungs of hyperoxic neonatal rat pups (FIGS. 32B and 32C).

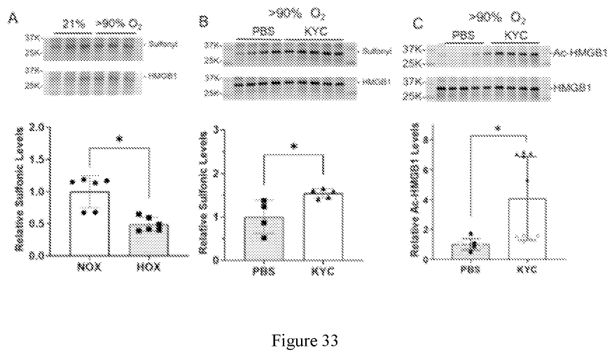

Effects of KYC Thiylation on HMGB1, Terminal HMGB1 Thiol Oxidation, and Shifts in Cell Sources for HMGB1 in Hyperoxic Lungs: Immunoblots of normoxic and hypoxic lung lysates revealed that lungs from normoxic pups had higher levels of sulfonyl HMGB1 than lungs from hyperoxic pups (FIG. 33A). Furthermore, KYC treatment of hyperoxic pups increased sulfonyl HMGB1 levels relative to sulfonyl HMGB1 levels in PBS-treated hyperoxic pups (FIG. 33B). These data suggest that KYC thiylation increased terminal HMGB1 thiol oxidation, which is well-known to inactivate HMGB1 [36]. These data support the idea that KYC thiylation breaks the destructive cycle in BPD by promoting HMGB1 terminal thiol oxidation. In addition to decreasing total HMGB1 levels in lung lysates (FIG. 27), non-depleting HMGB1 immuno-pulldown studies show that KYC treatment shifted the HMGB1 in lung lysates from the non-acetylated (HMGB1) to the lysyl-acetylated (Ac-HMGB1) isoform (FIG. 33C). These blots show that lung lysates from PBS-treated hyperoxic neonatal rat pups contained primarily non-acetylated-HMGB1, while lung lysates from KYC-treated hyperoxic neonatal rat pups contained predominantly lysyl-acetylated-HMGB1. As Ac-HMGB1 is secreted by activated immune cells, data in FIG. 33C are consistent with the idea that KYC treatment reduced pulmonary cell death, which is the most likely cellular source of non-acetylated HMGB1 in hyperoxic lungs.

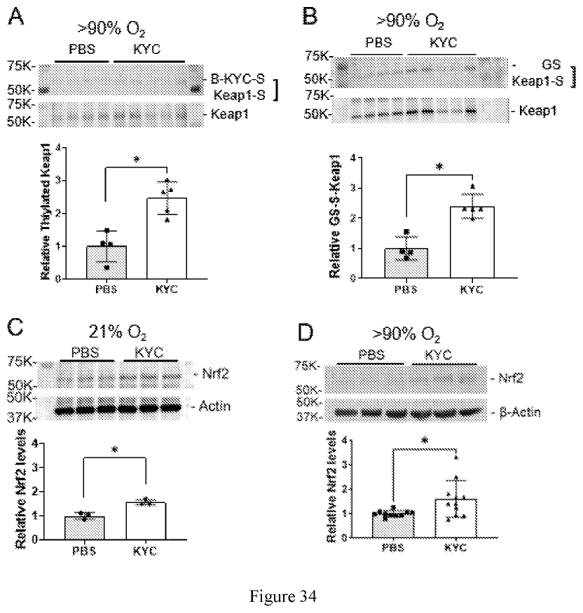

Effects of KYC on Keap1 KYC Thiylation and S-Glutathionylation and Modulation of Nrf2 in Normoxic and Hyperoxic Lungs: Band density analysis of streptavidin affinity and immunoblots revealed that KYC treatment of hyperoxic pups increased B-KYC thiylation of Keap1 and S-glutathionylation of Keap1 in lung lysates from hyperoxic pups (FIGS. 34A and 34B). As KYC increased B-KYC thiylation and S-glutathionylation of Keap1 in RLMVEC cultures even in the absence of MPO (FIG. 31), we examined KYC-dependent activation of Nrf2 in lung lysates from normoxic and hyperoxic pups. KYC treatment of normoxic pups increased Nrf2 activation in lung lysates (FIG. 34C) as well as in the lung lysates from hyperoxic neonatal rat pups (FIG. 34D). Data in FIG. 34D showing that KYC treatment increased Nrf2 activation in lung lysates from hyperoxic pups are in stark contrast with supplement data in Figure that shows that Nrf2 levels were markedly reduced in lung lysates from hyperoxic pups when compared to Nrf2 levels in lung lysates from normoxic pups.

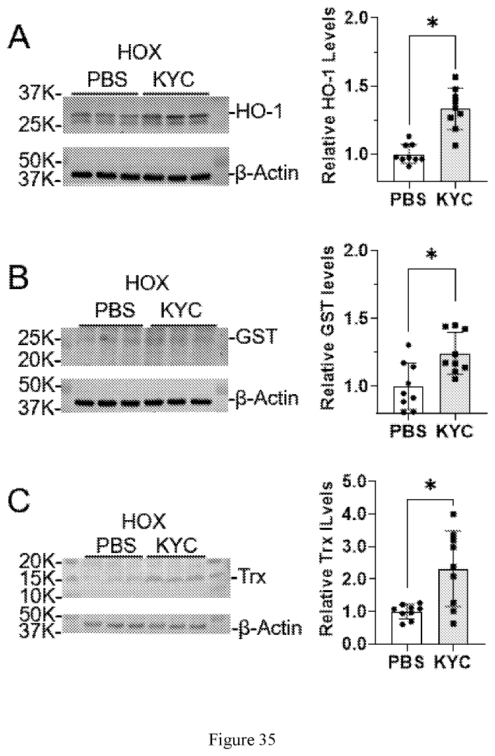

Effects of KYC Treatment on HO-1, GST, and Trx Expression in Hyperoxic Lungs. The immunoblots show that treating hyperoxic pups with KYC increased the expression of HO-1, GST, and Trx, which are all members of the antioxidant enzyme system known to be upregulated by Nrf2 (FIG. 35). These findings demonstrate that KYC effectively increases Nrf2-dependent antioxidant enzyme expression in the lungs of hyperoxic pups.

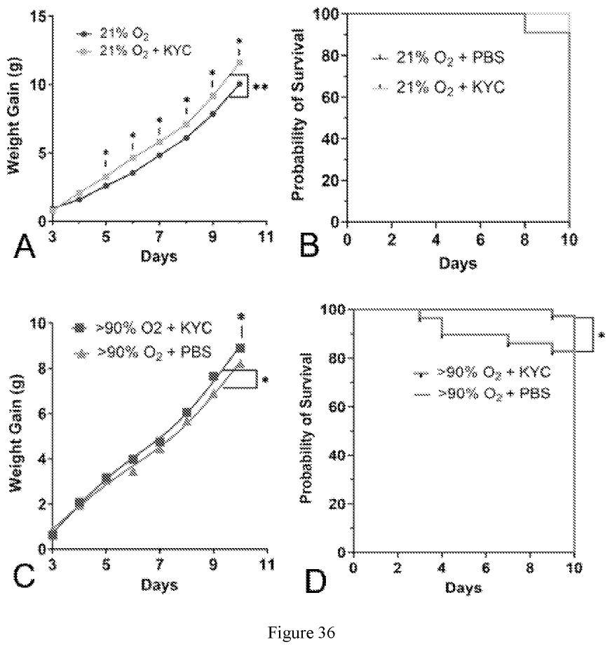

Effects of KYC on Weight Gain and Survival of Hyperoxic Pups: KYC-treated normoxic pups gained more weight than untreated normoxic pups based on curve analysis (FIG. 36A). Although KYC-treated hyperoxic pups tended to gain more weight than PBS-treated hyperoxic pups, time-dependent changes in weight gain were not significantly different, despite the means of these two test groups being significantly at P10 (FIG. 36C). These data are in contrast to the effects of KYC on normoxic pups that gained more weight than untreated normoxic pups (FIG. 36A). Possibly the number of data used for the PBS and KYC treated hyperoxic pups are underpowered for the variation. Additional weight data may be required to test the effects of hyperoxia on pup weight gain adequately. Concerning survival analysis, although more normoxic pups treated with KYC survived to P10 than untreated normoxic pups, no significant differences in survival were detected between these two groups. (FIG. 36B). In contrast, KYC-treated hyperoxic pups had a higher probability of surviving to P10 that PBS-treated hyperoxic pups (FIG. 36D).

Discussion

Our findings suggest that the MPO, HMGB1, and Nrf2 play essential roles in BPD. MPO, released from resident myeloid cells, plays an initiating role by amplifying the oxidative stress induced by hyperoxia. HMGB1, released from dead and dying lung cells, plays a propagating role by recruiting the myeloid cells that enter the interstitium of the lung and release MPO and increasing inflammation. The arrival and activation of the recruited myeloid cells induces a second wave of oxidative injury and cell death that is always greater than that which is induced initially by hyperoxia. Reduced Nrf2 activation plays a permissive role in BPD by preventing premature neonates who lack antioxidant enzymes from defending themselves against the oxidative stress mediated by chronic hyperoxia. In this way, blunted Nrf2 responses to the oxidative stress induced by hyperoxia allows hyperoxia to induce greater pulmonary cell injury and death [14], which result in proportionately greater release of HMGB1 [12]. Viewed in this fashion, failure to target all three mediators adequately permits the cycle to continue, resulting in varying degrees of BPD severity. Our findings suggest that BPD is caused by a destructive cycle that is mediated by oxidative stress and inflammation, where activation of one mediator results in the activation of a second mediator to worsen BPD. As myeloid cells are capable of releasing various mediators of oxidative stress and inflammation, and HMGB1 also increases vascular inflammation, it is likely that additional mediators of inflammation and oxidative stress be added to this list over time.

The mechanisms by which hyperoxia impairs lung development and increases BPD are complex. To begin to understand the cellular mechanisms mediating BPD, we first determined the effects of hyperoxia on the lungs of neonatal rat pups. After establishing baseline differences, we determined KYC's effects on preventing oxidative lung damage in the hyperoxic pups. Our studies showed that chronic hyperoxia decreased lung complexity and CD31 expression, a cellular biomarker for endothelial cell content. These data, taken together, suggest hyperoxia impaired angiogenesis. Hyperoxia increased lung MPO+ myeloid cell counts, MPO protein, and Cl-Tyr formation (a biomarker for HOCl-dependent oxidative damage). These data suggest that hyperoxia increased oxidative stress. In this context, it is significant that the increase in Cl-Tyr was paralleled by a second biomarker, 8-OH-dG, a biomarker of DNA oxidative damage, and that both biomarkers increased as lung development decreased and cell death increased [37]. Inflammation was assessed by immunoblotting, which showed that hyperoxia increased HMGB1, Cox1/Cox2, RAGE and TLR4. HMGB1 is a biomarker for lung cell death and injury, myeloid cell recruitment and vascular inflammation. Cox1/Cox2, RAGE and TLR4 are all biomarkers of inflammation. Finally, other immunoblots revealed that hyperoxia decreased Nrf2 activation. The inability of the lungs of neonatal rat pups to activate Nrf2 makes the pups more susceptible to oxidative stress (see supplemental).

Treating hyperoxic pups with KYC improved lung complexity, reduced oxidative stress and inflammation, and reversed the biomarkers for oxidative stress, DNA damage and inflammation in the hyperoxic pups' lungs. Thus, KYC treatment effectively reduced oxidative stress and inflammation, and restored lung development in the hyperoxic pups and, in so doing, likely increased survival.

The fact that KYC inhibits multiple mechanisms suggests that KYC is a systems pharmacology agent. In 2013, we reported that KYC not only inhibited MPO-dependent HOCl and nitrogen dioxide (NO₂°) production, but also increased MPO-dependent H₂O₂ consumption [27], which, all things being equal, should reduce oxidative stress. Our idea that KYC increases MPO-dependent H₂O₂ consumption is consistent with the report by Kettle and Winterbourn [20], who showed that tyrosine increases MPO-dependent catalase activity. In 2013, we also reported that MPO oxidized KYC to a KYC thiyl radical that prevented further oxidative damage by autoscavenging to form either a homodimer with a second KYC or a heterodimer with GSH [27]. As all thiols require activation before they can thiylate a thiol group or a protein cysteine, we reasoned that KYC should reduce oxidative stress in the very places that myeloid cells released MPO. Such a model implies that the tissues benefiting the most from KYC would be proximal to MPO. Although our intent in developing this model was to address non-specificity, having such a model demanded that us to take a closer look at KYC thiylation.

To better understand thiylation biotin-labeled KYC was added to RLMVEC cultures under baseline, H₂O₂-dependent, and MPO-dependent conditions of oxidative stress. At baseline KYC thiylation of RLMVEC proteins was low. However, under H₂O₂-dependent oxidative stress conditions, KYC thiylation of RLMVEC proteins was only slightly increased, the majority of RLMVEC proteins that were KYC thiylated were not heavily thiylated and were scattered throughout the full range of molecular weights. Under MPO-dependent oxidative stress conditions, the level of KYC thiylation of RLMVEC proteins was markedly increased throughout a wide range of molecular weights. These data support our model that KYC thiylation occurs predominantly in cells proximal to MPO. Although oxidative stress may increase KYC thiylation in a few cell proteins, the KYC thiylation that occurs is diffuse and not much more than that which occurs at baseline. Protein expression data show that Keap1 and Nrf2 were increased in RLMVEC cultures treated MPO+H₂O₂+B-KYC, while HMGB1, a likely injury mechanism, was increased in the RLMVEC cultures treated with H₂O₂-dependent oxidative stress+B-KYC but not in RLMVEC cultures treated with MPO+H₂O₂+B-KYC. These data support our idea that B-KYC is an MPO substrate that increases MPO-dependent catalase activity [20] and reduces oxidative injury and damage to RLMVEC cultures.

Previously we reported that KYC reduced MPO-dependent oxidative damage because KYC competes with native substrates, such as chloride and nitrite, that are oxidized to “toxic” oxidants. In 2013, we argued that KYC reduced MPO-dependent oxidative damage because the resultant KYC thiyl radical would autoscavenge to prevent radical propagation [27]. HMGB1 expression data in FIG. 29 suggests that MPO-dependent KYC thiylation protects RLMVEC against oxidative injury. To determine the radical mechanisms mediating MPO oxidation of KYC and KYC thiylation of HMGB1, we performed two experiments. In the first experiment, we trapped KYC thiyl radicals generated by MPO with DMPO (FIG. 30A), which confirms our conclusions in 2013 [27]. In the second experiment, we added recombinant HMGB1 to PBS containing MPO+H₂O₂+B-KYC and determined KYC thiylation by streptavidin affinity blotting. The affinity blot showed that MPO oxidizes KYC into a new product that thiylates MPO and HMGB1. Evidence for KYC binding MPO and HMGB1 via a disulfide bond comes from DTT reduction in band density for MPO and HMGB1 in the split sample. These two in vitro studies confirm that MPO oxidizes KYC into a thiyl radical (FIG. 30A) and that the MPO-generated B-KYC thiyl radical thiylates HMGB1 and MPO (FIG. 30B).

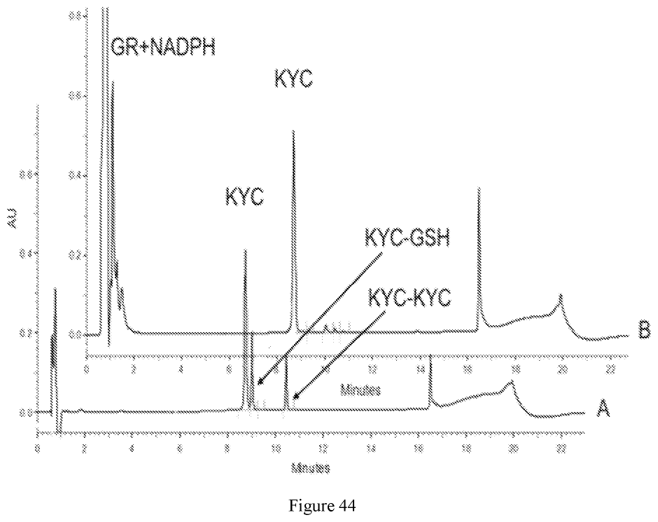

Although the exact mechanisms by which KYC thiylation of HMGB1 promotes terminal HMGB1 thiol oxidation remain unclear, some insight may be gained into the reactions promoting terminal thiol oxidation from in vitro studies. In vitro incubations show that GST reduces KYC disulfide to KYC monomers only in the presence of excess GSH (see supplemental data, FIG. 44). The importance of these observations is they confirm that GSH metabolizing enzymes do not metabolize heterodisulfides made with tripeptides other than GSH. Accordingly, in extracellular spaces, where GSH and other reducing agents may be in limited supply, KYC thiylation of HMGB1 will prevent HMGB1 from binding productively to RAGE and TLR4. If KYC-thiylated HMGB1 cannot bind productively to its receptors, then the KYC-thiylated HMGB1 will remain in the interstitium or circulation until its cysteines become fully oxidized, which is completely inactive [38]. In contrast, when KYC thiyl radical thiylates intracellular Keap-1, where GSH is in excess, KYC may be removed from the thiylated Keap-1 via GSH exchange. The exact mechanisms by which KYC thiylation of intracellular Keap-1 occurs and increases S-glutathionylation has not been fully examined. However, it is interesting to speculate that if KYC is removed from Keap-1 cysteines by GSH exchange, the KYC monomer will be released in the same location as the other reduced cysteines in Keap-1's cysteine clamp. Thus, the newly released monomeric KYC may thiylate a different cysteine in the clamp. Theoretically, such a KYC thiylation/GSH exchange process could be repeated until Keap-1 is fully glutathionylated, a process that should ensure Nrf2 activation. Additional studies are required to identify and define the mechanisms by which KYC treatment leads to KYC thiylation and, subsequently, S-glutathionylation of Keap-1 to activate Nrf2 and increase antioxidant gene expression. Data showing that KYC treatment of hyperoxic pups increases lung HO-1, GST, and Trx expression confirms that KYC increases Nrf2 activation, which increases antioxidant enzyme expression in lungs of hyperoxic pups. These data are in stark contrast to the effects of hyperoxia, which decreased Nrf2 activation, and increased 8-OH-dG in the lungs of hyperoxic pups (see supplemental FIGS. 29 and 25, respectively).

In animal studies relative rates of weight gain in neonatal rat pups provide an independent measure of animal development. Slower rates of weight gain suggest that the pups are under-developed or suffering from excessive stress. Survival curves reveal which experimental test groups are under stress or lacking the nutrition required to survive. In this context, it is important to note that KYC treatment of normoxic rat pups significantly increased weight (FIG. 15A). Although more KYC treated normoxic pups survived to P10 than untreated pups, the differences were not significant. In contrast, although the curves for hyperoxic pups treated with PBS and KYC are closer to each other statistical analysis clearly shows that the KYC-treated hyperoxic pups gained weight at a higher rate than the PBS-treated hyperoxic pups even though differences between means achieved statistical significance only on P10. KYC treatment significantly improved survival of the hyperoxic pups. These data are consistent with the marked increases in lung development, and reductions in pulmonary oxidative stress and inflammation mediated by KYC treatment.

In conclusion, BPD is caused by a destructive cycle mediated by oxidative stress and inflammation. The major mediators are the recruited myeloid cells that release MPO and generate H₂O₂ that activates MPO to generate toxic oxidants, HMGB1 released from dead and dying pulmonary cells, and inadequate Nrf2 activation. KYC is a novel systems pharmacology agent that we previously reported inhibits MPO toxic oxidant production and enhances MPO-dependent catalase activity [27]; and, that we now show here exploits MPO peroxidase activity to be oxidized into a KYC thiyl radical that thiylates and inactivates HMGB1, thiylates Keap-1 and increases Nrf2 activation. As Nrf2 mediates antioxidant enzyme expression, KYC reduces oxidative stress and inflammation and increases the lung's ability to protect itself against oxidative stress induced by hyperoxia to improve lung development even under conditions of chronic hyperoxia.

Supplemental Data

These supplemental data describe the effects of hyperoxia on oxidative stress, inflammation and bronchopulmonary dysplasia in the lungs of neonatal rat pups, and the effects of glutathione and glutathione metabolizing enzymes on reduction of KYC homo- and heterodisulfides.

Material and methods not provided here can be found in the manuscript.

Rats and Experimental Protocols: Sprague-Dawley rats were obtained from Envigo Bioproducts, Inc. (Madison, Wis.), and pregnancy was achieved naturally in our animal facility. Animal protocols were submitted to and approved by MCW's Institutional Animal Care and Use Committee and conformed to NIH Guide for the Care and Use of Laboratory Animals. The animals were housed in barrier cages with a 12-h dark-light cycle and were given free access to chow and water. The pups from two dams were mixed and randomly distributed to each nursing dam by balancing sex and size of the pups. The sex was determined by the relative distance between the genitalia and anus. The dam and pups were placed in cages in either room air (normoxia) or in >90% oxygen chamber (hyperoxia) from postnatal day 1 to day 10 (P1-P10) to induce BPD as previously reported [25, 26]. Oxygen concentrations were continuously monitored with an oxygen sensor (Reming Bioinstruments Co., Redfield, N.Y.).

Glutathione Reductase Reduction of KYC Homo- and Hetero-disulfide: Disulfide formation was induced by incubating either KYC alone (2.2 mM) or KYC with equal concentration glutathione (GSH) in PBS (pH 7.4) with shaking overnight at room temperature. The conditions used to assess the effects of glutathione reductase (GR) on KYC-KYC homodisulfide and KYC-GSH heterodisulfides were in the presence and absence of GSH as before [22, 31, 32]. Mixtures of KYC-KYC homodisulfide and KYC monomer and KYC-GSH heterodisulfide and GSH were incubated with 3.8 μM GR (Thermo-Fisher, Waltham, Mass.) in a 100 mM potassium phosphate buffer (pH 7.5) containing 2 mM EDTA, and 1 mM NADPH for 1 hr at room temperature with shaking. The reaction was halted by snap freezing at −80° C. Negative controls omitted either GR or NADPH. Samples were analyzed by HPLC (Beckman, Brea, Calif.) at the Blood Research Institute of Wisconsin Protein Core Lab. Samples were diluted 1:1 in 0.1% TFA, loaded on a Jupiter Proteo column (C-12 surface, dimensions: 4.6×50 mm, 4 μm particle size, Phenomenex, Torrence, Calif.) and run with a gradient (solvent B into solvent A) of 2-40% over 10 min with a flow rate of 1 ml/min and read using a UV detector at 220 nm. Solvent A was 0.08% TFA in HPLC grade H2O, and solvent B was 0.1% TFA in HPLC grade acetonitrile. Unless otherwise indicated, chemicals were obtained from Sigma-Aldrich (St. Louis, Mo.).

Results

Effects of Hyperoxia on BPD: Chronic hyperoxia increased the infiltration of MPO (+) myeloid cells in the lungs from neonatal pups compared with the number in lungs from normoxic pups (brown stained cells, FIG. 37A). Counts of brown-stained cells revealed that hyperoxia increased MPO positive (+) myeloid cell recruitment by 10- to 20-fold. Immunoblots for MPO confirmed histology findings. Quantification of band intensities showed hyperoxia increased MPO protein in the lungs of neonatal pups by 5-10-fold (FIG. 37B). Immunoblots for Cl-Tyr revealed that hyperoxic lungs experienced more MPO-dependent oxidative stress than normoxic lungs (FIG. 37C). Image analysis of the immunoblots revealed that hyperoxia increased Cl-Tyr formation by approximately 170%. These data demonstrate that chronic hyperoxia increases myeloid cell recruitment and infiltration into and MPO-dependent oxidative damage to neonatal lungs.

Effects of Hyperoxia on Lung Morphometrics: Hyperoxic lungs had decreased RAC, secondary septa, and blood vessel counts by approximately 27%, 52%, and 52%, respectively, and increased MLI values by approximately 40% compared with normoxic lungs (FIG. 38A-D). As many, if not all, of these architectural features in developing lungs are influenced by blood vessel growth, lysates of lung homogenates were immunoblotted for CD31 to assess the number of endothelial cells in the developing lung. The immunoblots showed that hyperoxia decreased CD31 in lung lysates by approximately 47% (FIG. 38E). These findings are consistent with the immunohistochemistry studies showing that chronic hyperoxia decreased the number of blood vessels and the complexity of vessel architecture in neonatal lungs.

Effects of Hyperoxia and KYC on Lung 8-OH-dG: Immunofluorescence staining shows that chronic hyperoxia increased the fluorescent density of 8-OH-dG in the stained cells by 1.8-fold (FIG. 39).

Effects of Hyperoxia and KYC on Lung Cox1/Cox2: Immunoblots for Cox1 and Cox2 show that chronic hyperoxia increased Cox1 slightly and Cox2 markedly in the lungs of neonatal rat pups (FIG. 40).

Effects of Hyperoxia on Lung HMGB1: Immunoblots show that chronic hyperoxia increased HMGB1 levels in lung homogenates by 170% compared with HMGB1 levels in lung homogenates from normoxic pups (FIG. 41).

Effects of Hyperoxia on RAGE and TLR4 Expression in Lungs of Hyperoxic Neonatal Rat Pups: Hyperoxia increased RAGE and TLR4 in the lungs of neonatal rat pups. Hyperoxia increased the expression of RAGE by approximately 130% (FIG. 42A) and TLR4 by approximately 160% (FIG. 42B).

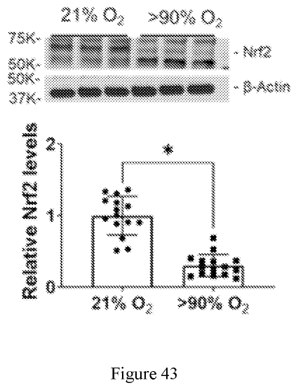

Effects of Hyperoxia on Nrf2 Activation in Lungs of Neonatal Rat Pups: Hyperoxia Decreased Nrf2 Activation in the Lungs of Neonatal Rat Pups. Hyperoxia decreased Nrf2 activation by approximately 70% (FIG. 43).

Effects of Glutathione Reductase on Reduction of KYC-KYC Homo-disulfide and KYC-GSH Hetero-disulfide to KYC Monomer: One of the mechanisms by which KYC is hypothesized to reduce MPO-dependent oxidant production is by forming KYC homodimers (KYC-KYC) that can be reduced to an active KYC monomeric inhibitor by GSH [22]. GSH homodimer (GS-SG) can be reduced to monomeric GSH by glutathione reductase (GR) [35]. GSH homodimers can also undergo thiol exchange to form mixed heterodimers (GS-SR). The KYC-KYC and KYC-GSH mixtures were incubated with a GR\NADPH reaction mixture as described in methods and KYC monomers determined by HPLC as before [22]. The HPLC tracings in FIG. 29 shows that the GR\NADPH reaction mixtures reduced KYC-KYC and KYC-GSH when GSH was present to KYC monomers. As a GR\NADPH mixture in the absence of GSH did not reduce KYC homodimers to KYC monomers (data not shown), GSH thiol exchange is required for GR to be able to reduce KYC homo-disulfides to an active KYC monomer for continued inhibition of MPO toxic oxidant production.

### REFERENCES CITED

1. Ali Z, Schmidt P, Dodd J, and Jeppesen D L. Bronchopulmonary dysplasia: a review. Arch Gynecol Obstet 288: 325-333, 2013.
2. Auten R L, Jr., Mason S N, Tanaka D T, Welty-Wolf K, and Whorton M H. Anti-neutrophil chemokine preserves alveolar development in hyperoxia-exposed newborn rats. Am J Physiol Lung Cell Mol Physiol 281: L336-344, 2001.
3. Auten R L, Richardson R M, White J R, Mason S N, Vozzelli M A, and Whorton M H. Nonpeptide CXCR2 antagonist prevents neutrophil accumulation in hyperoxia-exposed newborn rats. J Pharmacol Exp Ther 299: 90-95, 2001.
4. Baldus S, Eiserich J P, Brennan M L, Jackson R M, Alexander C B, and Freeman B A. Spatial mapping of pulmonary and vascular nitrotyrosine reveals the pivotal role of myeloperoxidase as a catalyst for tyrosine nitration in inflammatory diseases. Free Radic Biol Med 33: 1010, 2002.
5. Bauer E M, Shapiro R, Billiar T R, and Bauer P M. High mobility group Box 1 inhibits human pulmonary artery endothelial cell migration via a Toll-like receptor 4- and interferon response factor 3-dependent mechanism(s). J Biol Chem 288: 1365-1373, 2013.
6. Bauer E M, Shapiro R, Zheng H, Ahmad F, Ishizawar D, Comhair S A, Erzurum S C, Billiar T R, and Bauer P M. High Mobility Group Box 1 Contributes to the Pathogenesis of Experimental Pulmonary Hypertension via Activation of Toll-like Receptor 4. Mol Med 18: 1509-1518, 2013.
7. Belcher J D, Chen C, Nguyen J, Zhang P, Abdulla F, Nguyen P, Killeen T, Xu P, O'Sullivan G, Nath K A, and Vercellotti G M. Control of Oxidative Stress and Inflammation in Sickle Cell Disease with the Nrf2 Activator Dimethyl Fumarate. Antioxid Redox Signal 2016.
8. Berkelhamer S K, and Farrow K N. Developmental regulation of antioxidant enzymes and their impact on neonatal lung disease. Antioxid Redox Signal 21: 1837-1848, 2014.
9. Berthelot F, Fattoum L, Casulli S, Gozlan J, Marechal V, and Elbim C. The effect of HMGB1, a damage-associated molecular pattern molecule, on polymorphonuclear neutrophil migration depends on its concentration. J Innate Immun 4: 41-58, 2012.
10. Buesing K L, Densmore J C, Kaul S, Pritchard K A, Jr., Jarzembowski J A, Gourlay D M, and Oldham K T. Endothelial microparticles induce inflammation in acute lung injury. J Surg Res 166: 32-39, 2011.
11. Burner U, Furtmuller P G, Kettle A J, Koppenol W H, and Obinger C. Mechanism of reaction of myeloperoxidase with nitrite. J Biol Chem 275: 20597-20601, 2000.
12. Carlberg I, and Mannervik B. Reduction of 2,4,6-trinitrobenzenesulfonate by glutathione reductase and the effect of NADP+ on the electron transfer. J Biol Chem 261: 1629-1635, 1986.
13. Coalson J J, Winter V T, Siler-Khodr T, and Yoder B A. Neonatal chronic lung disease in extremely immature baboons. Am J Respir Crit Care Med 160: 1333-1346, 1999.
14. Davidson L M, and Berkelhamer S K. Bronchopulmonary Dysplasia: Chronic Lung Disease of Infancy and Long-Term Pulmonary Outcomes. J Clin Med 6: 2017.
15. Deng H, Mason S N, and Auten R L, Jr. Lung inflammation in hyperoxia can be prevented by antichemokine treatment in newborn rats. Am J Respir Crit Care Med 162: 2316-2323, 2000.
16. Deng Y, Yang Z, Gao Y, Xu H, Zheng B, Jiang M, Xu J, He Z, and Wang X. Toll-like receptor 4 mediates acute lung injury induced by high mobility group box-1. PLoS One 8: e64375, 2013.
17. El Kebir D, Jozsef L, Pan W, and Filep J G. Myeloperoxidase delays neutrophil apoptosis through CD11b/CD18 integrins and prolongs inflammation. Circ Res 103: 352-359, 2008.
18. Entezari M, Javdan M, Antoine D J, Morrow D M, Sitapara R A, Patel V, Wang M, Sharma L, Gorasiya S, Zur M, Wu W, Li J, Yang H, Ashby C R, Thomas D, Wang H, and Mantell L L. Inhibition of extracellular HMGB1 attenuates hyperoxia-induced inflammatory acute lung injury. Redox Biol 2: 314-322, 2014.
19. Gaut J P, Byun J, Tran H D, Lauber W M, Carroll J A, Hotchkiss R S, Belaaouaj A, and Heinecke J W. Myeloperoxidase produces nitrating oxidants in vivo. J Clin Invest 109: 1311-1319, 2002.
20. Gitto E, Reiter R J, Sabatino G, Buonocore G, Romeo C, Gitto P, Bugge C, Trimarchi G, and Barberi I. Correlation among cytokines, bronchopulmonary dysplasia and modality of ventilation in preterm newborns: improvement with melatonin treatment. J Pineal Res 39: 287-293, 2005.
21. Hsia C C, Hyde D M, Ochs M, Weibel E R, and Structure AEJTFoQAoL. An official research policy statement of the American Thoracic Society/European Respiratory Society: standards for quantitative assessment of lung structure. Am J Respir Crit Care Med 181: 394-418, 2010.
22. Jain D, and Bancalari E. Prevention of bronchopulmonary dysplasia: current strategies. Zhongguo Dang Dai Er Ke Za Zhi 19: 841-851, 2017.
23. Jeitner T M, Kalogiannis M, Krasnikov B F, Gomolin I, Peltier M R, and Moran G R. Linking Inflammation and Parkinson Disease: Hypochlorous Acid Generates Parkinsonian Poisons. Toxicol Sci 151: 388-402, 2016.
24. Jensen E A, Foglia E E, and Schmidt B. Evidence-Based Pharmacologic Therapies for Prevention of Bronchopulmonary Dysplasia: Application of the Grading of Recommendations Assessment, Development, and Evaluation Methodology. Clin Perinatol 42: 755-779, 2015.
25. Jing X, Huang Y W, Jarzembowski J, Shi Y, Konduri G G, and Teng R J. Caffeine ameliorates hyperoxia-induced lung injury by protecting GCH1 function in neonatal rat pups. Pediatr Res 82: 483-489, 2017.
26. Kalikkot Thekkeveedu R, Guaman M C, and Shivanna B. Bronchopulmonary dysplasia: A review of pathogenesis and pathophysiology. Respir Med 132: 170-177, 2017.
27. Klinke A, Berghausen E, Friedrichs K, Molz S, Lau D, Remane L, Berlin M, Kaltwasser C, Adam M, Mehrkens D, Mollenhauer M, Manchanda K, Ravekes T, Heresi G A, Aytekin M, Dweik R A, Hennigs J K, Kubala L, Michaelsson E, Rosenkranz S, Rudolph T K, Hazen S L, Klose H, Schermuly R T, Rudolph V, and Baldus S. Myeloperoxidase aggravates pulmonary arterial hypertension by activation of vascular Rho-kinase. JCI Insight 3: 2018.
28. Kremserova S, Perecko T, Soucek K, Klinke A, Baldus S, Eiserich J P, and Kubala L. Lung Neutrophilia in Myeloperoxidase Deficient Mice during the Course of Acute Pulmonary Inflammation. Oxid Med Cell Longev 2016: 5219056, 2016.
29. Lee C C, Lai Y T, Chang H T, Liao J W, Shyu W C, Li C Y, and Wang C N. Inhibition of high-mobility group box 1 in lung reduced airway inflammation and remodeling in a mouse model of chronic asthma. Biochem Pharmacol 86: 940-949, 2013.
30. Little M J, Aubry N, Beaudoin M E, Goudreau N, and LaPlante S R. Quantifying trifluoroacetic acid as a counterion in drug discovery by 19F NMR and capillary electrophoresis. J Pharm Biomed Anal 43: 1324-1330, 2007.
31. Mathew O P. Apnea of prematurity: pathogenesis and management strategies. J Perinatol 31: 302-310, 2011.
32. McEvoy C T, Jain L, Schmidt B, Abman S, Bancalari E, and Aschner J L. Bronchopulmonary dysplasia: NHLBI Workshop on the Primary Prevention of Chronic Lung Diseases. Ann Am Thorac Soc 11 Suppl 3: S146-153, 2014.
33. Ogawa Y, Tasaka S, Yamada W, Saito F, Hasegawa N, Miyasho T, and Ishizaka A. Role of Toll-like receptor 4 in hyperoxia-induced lung inflammation in mice. Inflamm Res 56: 334-338, 2007.
34. Ogden B E, Murphy S, Saunders G C, and Johnson J D. Lung lavage of newborns with respiratory distress syndrome. Prolonged neutrophil influx is associated with bronchopulmonary dysplasia. Chest 83: 31S-33S, 1983.
35. Orlova V V, Choi E Y, Xie C, Chavakis E, Bierhaus A, Ihanus E, Ballantyne C M, Gahmberg C G, Bianchi M E, Nawroth P P, and Chavakis T. A novel pathway of HMGB1-mediated inflammatory cell recruitment that requires Mac-1-integrin. EMBO J 26: 1129-1139, 2007.
36. Palladino E N D, Hartman C L, Albert C J, and Ford D A. The chlorinated lipidome originating from myeloperoxidase-derived HOCl targeting plasmalogens: Metabolism, clearance, and biological properties. Arch Biochem Biophys 641: 31-38, 2018.
37. Palladino E N D, Katunga L A, Kolar G R, and Ford D A. 2-Chlorofatty acids: lipid mediators of neutrophil extracellular trap formation. J Lipid Res 59: 1424-1432, 2018.
38. Pan L, Fu J H, Xue X D, Xu W, Zhou P, and Wei B. Melatonin protects against oxidative damage in a neonatal rat model of bronchopulmonary dysplasia. World J Pediatr 5: 216-221, 2009.
39. Pasha A B, Chen X Q, and Zhou G P. Bronchopulmonary dysplasia: Pathogenesis and treatment. Exp Ther Med 16: 4315-4321, 2018.
40. Poggi C, and Dani C. Antioxidant strategies and respiratory disease of the preterm newborn: an update. Oxid Med Cell Longev 2014: 721043, 2014.
41. Qiao J, Chen L, Huang X, and Guo F. Effects of nebulized N-acetylcystein on the expression of HMGB1 and RAGE in rats with hyperoxia-induced lung injury. J Cell Physiol 2018.
42. Radi R. Nitric oxide, oxidants, and protein tyrosine nitration. Proc Natl Acad Sci USA 101: 4003-4008, 2004.
43. Rahman I, Kode A, and Biswas S K. Assay for quantitative determination of glutathione and glutathione disulfide levels using enzymatic recycling method. Nat Protoc 1: 3159-3165, 2006.
44. Reynolds P R, Schmitt R E, Kasteler S D, Sturrock A, Sanders K, Bierhaus A, Nawroth P P, Paine R, 3rd, and Hoidal J R. Receptors for advanced glycation end-products targeting protect against hyperoxia-induced lung injury in mice. Am J Respir Cell Mol Biol 42: 545-551, 2010.
45. Schindhelm R K, van der Zwan L P, Teerlink T, and Scheffer P G. Myeloperoxidase: a useful biomarker for cardiovascular disease risk stratification? Clin Chem 55: 1462-1470, 2009.
46. Schmidt B, Roberts R S, Davis P, Doyle L W, Barrington K J, Ohlsson A, Solimano A, Tin W. Caffeine for Apnea of Prematurity Trial G. Caffeine therapy for apnea of prematurity. N Engl J Med354: 2112-2121, 2006.
47. Stone Jr C A, McEvoy C T, Aschner J L, Kirk A, Rosas-Salazar C, Cook-Mills J M, Moore P E, Walsh W F, and Hartert T V. Update on Vitamin E and Its Potential Role in Preventing or Treating Bronchopulmonary Dysplasia. Neonatology 113: 366-378, 2018.
48. Suleymanoglu S, Cekmez F, Cetinkaya M, Tayman C, Canpolat F E, Kafa I M, Tanju A, Arziman I K, Fidanci M, Tunc T, and Sarici S U. Protective effects of melatonin therapy in model for neonatal hyperoxic lung injury. Altern Ther Health Med 20: 24-29, 2014.
49. Surate Solaligue D E, Rodriguez-Castillo J A, Ahlbrecht K, and Morty R E. Recent advances in our understanding of the mechanisms of late lung development and bronchopulmonary dysplasia. Am J Physiol Lung Cell Mol Physiol 313: L1101-L1153, 2017.
50. Taha D, Kirkby S, Nawab U, Dysart K C, Genen L, Greenspan J S, and Aghai Z H. Early caffeine therapy for prevention of bronchopulmonary dysplasia in preterm infants. J Matern Fetal Neonatal Med 27: 1698-1702, 2014.
51. Teng R J, Jing X, Michalkiewicz T, Afolayan A J, Wu T J, and Konduri G G. Attenuation of endoplasmic reticulum stress by caffeine ameliorates hyperoxia-induced lung injury. Am J Physiol Lung Cell Mol Physiol 312: L586-L598, 2017.
52. Wang J. Neutrophils in tissue injury and repair. Cell Tissue Res 371: 531-539, 2018.
53. Wang J, Lofgren S, Dong X, Galichanin K, and Soderberg P G. Evolution of light scattering and redox balance in the rat lens after in vivo exposure to close-to-threshold dose ultraviolet radiation. Acta Ophthalmol 88: 779-785, 2010.
54. Winterbourn C C, and Kettle A J. Biomarkers of myeloperoxidase-derived hypochlorous acid. Free Radic Biol Med 29: 403-409, 2000.
55. Yu B, Li X, Wan Q, Han W, Deng C, and Guo C. High-Mobility Group Box-1 Protein Disrupts Alveolar Elastogenesis of Hyperoxia-Injured Newborn Lungs. 36: 159-168, 2016.
56. Yu G, Liang Y, Huang Z, Jones D W, Pritchard K A, Jr., and Zhang H. Inhibition of myeloperoxidase oxidant production by N-acetyl lysyltyrosylcysteine amide reduces brain damage in a murine model of stroke. J Neuroinflammation 13: 119, 2016.
57. Yu G, Liang Y, Zheng S, and Zhang H. Inhibition of Myeloperoxidase by N-Acetyl Lysyltyrosylcysteine Amide Reduces Oxidative Stress-Mediated Inflammation, Neuronal Damage, and Neural Stem Cell Injury in a Murine Model of Stroke. J Pharmacol Exp Ther 364: 311-322, 2018.
58. Yu G, Zheng S, and Zhang H. Inhibition of myeloperoxidase by N-acetyl lysyltyrosylcysteine amide reduces experimental autoimmune encephalomyelitis-induced injury and promotes oligodendrocyte regeneration and neurogenesis in a murine model of progressive multiple sclerosis. Neuroreport 29: 208-213, 2018.
59. Zhang H, Jing X, Shi Y, Xu H, Du J, Guan T, Weihrauch D, Jones D W, Wang W, Gourlay D, Oldham K T, Hillery C A, and Pritchard K A, Jr. N-acetyl lysyltyrosylcysteine amide inhibits myeloperoxidase, a novel tripeptide inhibitor. J Lipid Res 54: 3016-3029, 2013.
60. Zhang H, Ray A, Miller N M, Hartwig D, Pritchard K A, Jr., and Dittel B N. Inhibition of Myeloperoxidase at the Peak of Experimental Autoimmune Encephalomyelitis Restores Blood-Brain-Barrier Integrity and Ameliorates Disease Severity. J Neurochem 826-836, 2016.
61. Zhang H, Xu H, Weihrauch D, Jones D W, Jing X, Shi Y, Gourlay D, Oldham K T, Hillery C A, and Pritchard K A, Jr. Inhibition of myeloperoxidase decreases vascular oxidative stress and increases vasodilatation in sickle cell disease mice. J Lipid Res 54: 3009-3015, 2013.

The invention is not limited to the embodiments set forth in this disclosure for illustration but includes everything that is within the scope of the appended claims.
62. C. T. McEvoy, L. Jain, B. Schmidt, S. Abman, E. Bancalari, J. L. Aschner, Bronchopulmonary dysplasia: NHLBI Workshop on the Primary Prevention of Chronic Lung Diseases, Ann Am Thorac Soc 11 Suppl 3 (2014) S146-53.
63. L. M. Davidson, S. K. Berkelhamer, Bronchopulmonary Dysplasia: Chronic Lung Disease of Infancy and Long-Term Pulmonary Outcomes, J Clin Med 6(1) (2017).
64. L. C. Eldredge, P. M. Treuting, A. M. Manicone, S. F. Ziegler, W. C. Parks, J. K. McGuire, CD11b(+) Mononuclear Cells Mitigate Hyperoxia-Induced Lung Injury in Neonatal Mice, Am J Respir Cell Mol Biol 54(2) (2016) 273-83.
65. A. Harijith, V. Natarajan, P. Fu, The Role of Nicotinamide Adenine Dinucleotide Phosphate Oxidases in Lung Architecture Remodeling, Antioxidants (Basel) 6(4) (2017).
66. I. Al Ghouleh, N. K. Khoo, U. G. Knaus, K. K. Griendling, R. M. Touyz, V. J. Thannickal, A. Barchowsky, W. M. Nauseef, E. E. Kelley, P. M. Bauer, V. Darley-Usmar, S. Shiva, E. Cifuentes-Pagano, B. A. Freeman, M. T. Gladwin, P. J. Pagano, Oxidases and peroxidases in cardiovascular and lung disease: new concepts in reactive oxygen species signaling, Free Radic Biol Med 51(7) (2011) 1271-88.
67. A. A. Khan, M. A. Alsahli, A. H. Rahmani, Myeloperoxidase as an Active Disease Biomarker: Recent Biochemical and Pathological Perspectives, Med Sci (Basel) 6(2) (2018).
68. J. Byun, D. M. Mueller, J. S. Fabjan, J. W. Heinecke, Nitrogen dioxide radical generated by the myeloperoxidase-hydrogen peroxide-nitrite system promotes lipid peroxidation of low density lipoprotein, FEBS Lett 455(3) (1999) 243-6.
69. S. S. Lange, K. M. Vasquez, HMGB1: the jack-of-all-trades protein is a master DNA repair mechanic, Mol Carcinog 48(7) (2009) 571-80.
70. S. Kim, S. Y. Kim, J. P. Pribis, M. Lotze, K. P. Mollen, R. Shapiro, P. Loughran, M. J. Scott, T. R. Billiar, Signaling of High Mobility Group Box 1 (HMGB1) through Toll-like Receptor 4 in Macrophages Requires CD14, Mol Med (2013).
71. H. Yang, H. Wang, S. S. Chavan, U. Andersson, High Mobility Group Box Protein 1 (HMGB1): The Prototypical Endogenous Danger Molecule, Mol Med 21 Suppl 1 (2015) S6-S12.
72. D. S. Bonikos, K. G. Bensch, W. H. Northway, Jr., D. K. Edwards, Bronchopulmonary dysplasia: the pulmonary pathologic sequel of necrotizing bronchiolitis and pulmonary fibrosis, Hum Pathol 7(6) (1976) 643-66.
73. Z. H. Aghai, J. G. Saslow, C. Meniru, C. Porter, R. Eydelman, V. Bhat, G. Stahl, S. Sannoh, K. Pyon, C. Hewitt, V. Bhandari, High-mobility group box-1 protein in tracheal aspirates from premature infants: relationship with bronchopulmonary dysplasia and steroid therapy, J Perinatol 30(9) (2010) 610-5.
74. A. P. Autor, L. Frank, R. J. Roberts, Developmental Characteristics of Pulmonary Superoxide Dismutase: Relationship to Idiopathic Respiratory Distress Syndrome, Pediatric Research 10(3) (1976) 154-158.
75. S. K. Berkelhamer, K. N. Farrow, Developmental regulation of antioxidant enzymes and their impact on neonatal lung disease, Antioxid Redox Signal 21(13) (2014) 1837-48.
76. H. Y. Cho, A. E. Jedlicka, S. P. Reddy, T. W. Kensler, M. Yamamoto, L. Y. Zhang, S. R. Kleeberger, Role of NRF2 in protection against hyperoxic lung injury in mice, Am J Respir Cell Mol Biol 26(2) (2002) 175-82
77. H. Y. Cho, S. P. Reddy, S. R. Kleeberger, Nrf2 defends the lung from oxidative stress, Antioxid Redox Signal 8(1-2) (2006) 76-87.
78. H.-Y. Cho, X. Wang, J. Li, D. A. Bell, S. R. Kleeberger, Potential therapeutic targets in Nrf2-dependent protection against neonatal respiratory distress disease predicted by cDNA microarray analysis and bioinformatics tools, Current opinion in toxicology 1 (2016) 125-133.
79. H. Y. Cho, A. E. Jedlicka, S. P. Reddy, L. Y. Zhang, T. W. Kensler, S. R. Kleeberger, Linkage analysis of susceptibility to hyperoxia. Nrf2 is a candidate gene, Am J Respir Cell Mol Biol 26(1) (2002) 42-51.
80. H. Y. Cho, S. R. Kleeberger, Association of Nrf2 with airway pathogenesis: lessons learned from genetic mouse models, Archives of toxicology 89(11) (2015) 1931-57.
81. A. J. Kettle, C. C. Winterbourn, A kinetic analysis of the catalase activity of myeloperoxidase, Biochemistry 40(34) (2001) 10204-12.
82. H. Zhang, H. Xu, D. Weihrauch, D. W. Jones, X. Jing, Y. Shi, D. Gourlay, K. T. Oldham, C. A. Hillery, K. A. Pritchard, Jr., Inhibition of myeloperoxidase decreases vascular oxidative stress and increases vasodilatation in sickle cell disease mice, J Lipid Res 54(11) (2013) 3009-3015.
83. G. Yu, Y. Liang, Z. Huang, D. W. Jones, K. A. Pritchard, Jr., H. Zhang, Inhibition of myeloperoxidase oxidant production by N-acetyl lysyltyrosylcysteine amide reduces brain damage in a murine model of stroke, J Neuroinflammation 13(1) (2016) 119.
84. H. Zhang, A. Ray, N. M. Miller, D. Hartwig, K. A. Pritchard, Jr., B. N. Dittel, Inhibition of Myeloperoxidase at the Peak of Experimental Autoimmune Encephalomyelitis Restores Blood-Brain-Barrier Integrity and Ameliorates Disease Severity, J Neurochem (2016) 826-36.
85. M. J. Little, N. Aubry, M. E. Beaudoin, N. Goudreau, S. R. LaPlante, Quantifying trifluoroacetic acid as a counterion in drug discovery by 19F NMR and capillary electrophoresis, J Pharm Biomed Anal 43(4) (2007) 1324-30.
86. X. Jing, Y. W. Huang, J. Jarzembowski, Y. Shi, G. G. Konduri, R. J. Teng, Caffeine ameliorates hyperoxia-induced lung injury by protecting GCH1 function in neonatal rat pups, Pediatr Res 82(3) (2017) 483-489.
87. R. J. Teng, X. Jing, T. Michalkiewicz, A. J. Afolayan, T. J. Wu, G. G. Konduri, Attenuation of endoplasmic reticulum stress by caffeine ameliorates hyperoxia-induced lung injury, Am J Physiol Lung Cell Mol Physiol 312(5) (2017) L586-L598.
88. H. Zhang, X. Jing, Y. Shi, H. Xu, J. Du, T. Guan, D. Weihrauch, D. W. Jones, W. Wang, D. Gourlay, K. T. Oldham, C. A. Hillery, K. A. Pritchard, Jr., N-acetyl lysyltyrosylcysteine amide inhibits myeloperoxidase, a novel tripeptide inhibitor, J Lipid Res 54(11) (2013) 3016-3029.
89. A. L. Rymaszewski, E. Tate, J. P. Yimbesalu, A. E. Gelman, J. A. Jarzembowski, H. Zhang, K. A. Pritchard, Jr., H. G. Vikis, The role of neutrophil myeloperoxidase in models of lung tumor development, Cancers 6(2) (2014) 1111-27.
90. G. Yu, Y. Liang, S. Zheng, H. Zhang, Inhibition of Myeloperoxidase by N-Acetyl Lysyltyrosylcysteine Amide Reduces Oxidative Stress-Mediated Inflammation, Neuronal Damage, and Neural Stem Cell Injury in a Murine Model of Stroke, J Pharmacol Exp Ther 364(2) (2018) 311-322.
91. G. Yu, S. Zheng, H. Zhang, Inhibition of myeloperoxidase by N-acetyl lysyltyrosylcysteine amide reduces experimental autoimmune encephalomyelitis-induced injury and promotes oligodendrocyte regeneration and neurogenesis in a murine model of progressive multiple sclerosis, Neuroreport 29(3) (2018) 208-213.
92. D. Weihrauch, D. P. Martin, D. Jones, J. Krolikowski, J. Struve, S. Naylor, K. A. Pritchard, Jr., Inhibition of myeloperoxidase increases revascularization and improves blood flow in a diabetic mouse model of hindlimb ischaemia, Diab Vasc Dis Res 17(3) (2020) 1479164120907971.
93. H. Xu, N. J. Wandersee, Y. Guo, D. W. Jones, S. L. Holzhauer, M. S. Hanson, E. Machogu, D. C. Brousseau, N. Hogg, J. C. Densmore, S. Kaul, C. A. Hillery, K. A. Pritchard, Jr., Sickle cell disease increases high mobility group box 1: a novel mechanism of inflammation, Blood 124(26) (2014) 3978-81.
94. C. C. Hsia, D. M. Hyde, M. Ochs, E. R. Weibel, How to measure lung structure—what for? On the “Standards for the quantitative assessment of lung structure”, Respir Physiol Neurobiol 171(2) (2010) 72-4.
95. H. Zhang, Y. Xu, J. Joseph, B. Kalyanaraman, Influence of intramolecular electron transfer mechanism in biological nitration, nitrosation, and oxidation of redox-sensitive amino acids, Methods Enzymol 440 (2008) 65-94.
96. H. Yang, H. S. Hreggvidsdottir, K. Palmblad, H. Wang, M. Ochani, J. Li, B. Lu, S. Chavan, M. Rosas-Ballina, Y. Al-Abed, S. Akira, A. Bierhaus, H. Erlandsson-Harris, U. Andersson, K. J. Tracey, A critical cysteine is required for HMGB1 binding to Toll-like receptor 4 and activation of macrophage cytokine release, Proc Natl Acad Sci USA 107(26) (2010) 11942-7.
97. H. Yang, P. Lundback, L. Ottosson, H. Erlandsson-Harris, E. Venereau, M. E. Bianchi, Y. Al-Abed, U. Andersson, K. J. Tracey, D. J. Antoine, Redox modification of cysteine residues regulates the cytokine activity of high mobility group box-1 (HMGB1), Mol Med 18 (2012) 250-9.
98. H. Y. Cho, B. van Houten, X. Wang, L. Miller-DeGraff, J. Fostel, W. Gladwell, L. Perrow, V. Panduri, L. Kobzik, M. Yamamoto, D. A. Bell, S. R. Kleeberger, Targeted deletion of nrf2 impairs lung development and oxidant injury in neonatal mice, Antioxid Redox Signal 17(8) (2012) 1066-82.
99. E. Venereau, M. Casalgrandi, M. Schiraldi, D. J. Antoine, A. Cattaneo, F. De Marchis, J. Liu, A. Antonelli, A. Preti, L. Raeli, S. S. Shams, H. Yang, L. Varani, U. Andersson, K. J. Tracey, A. Bachi, M. Uguccioni, M. E. Bianchi, Mutually exclusive redox forms of HMGB1 promote cell recruitment or proinflammatory cytokine release, J Exp Med 209(9) (2012) 1519-28.

1. A systems chemico-pharmacology drug (SCPD) for treating a disease or condition characterized by an increased peroxidase activity in a subject, the SCPD configured such that:
i. the SCPD interacts with a first target that comprises a druggable biomolecular site;
ii. the SCPD modifies the properties and functions of the first target and is itself chemically modified and activated, and
ii. the chemically modified/activated SCPD interacts with one or more second targets, thus modifying each of the second targets' functions;
whereby the disease or condition is treated in the subject.

2. The SCPD of claim 1, wherein the druggable biomolecular site is a protein, a peptide, a DNA segment, an RNA segment, or a metabolite.

3. The SCPD of claim 2, wherein the druggable biomolecular site is an active site of a peroxidase.

4. The SCPD of claim 3, wherein the peroxidase is a myeloperoxidase (MPO) or eosinophil peroxidase (EPO).

5. The SCPD of claim 1, wherein the SCPD is an agonist or an antagonist of the first target.

6. The SCPD of claim 1, wherein the SCPD is chemically modified after administration to a subject by being oxidized, being reduced, forming a radical, forming a salt, or undergoing energetic excitation.

7. The SCPD of claim 6, wherein undergoing energetic excitation comprises electron transfer excitation.

8. The SCPD of claim 6, wherein the chemically modified SCPD comprises a stabilized free radical.

9. The SCPD of claim 8, wherein the stabilized free radical is activated such that it is capable of reacting with each of the one or more second targets.

10. The SCPD of claim 8, wherein the chemically modified version of the SCPD is capable of auto-scavenging by forming a homodimer of chemically modified SCPDs via an oxidized linkage.

11. The SCPD of claim 8, wherein the chemically modified SCPD is capable of scavenging by forming a heterodimer with said second target via an oxidized linkage.

12. The SCPD of claim 9, wherein the at least one of the one or more second targets is a peptide or protein.

13. The SCPD of claim 12, wherein the peptide or protein is a pro-inflammatory peptide or protein, and wherein the peptide or protein's function is modified to reduce its pro-inflammatory activity.

14. The SCPD of claim 13, wherein the pro-inflammatory peptide is glutathione.

15. The SCPD of claim 13, wherein the pro-inflammatory protein is HMGB1, RAGE, TRL4, NRf2 or KEAP-1.

16. The SCPD of claim 1, wherein the SCPD has the peptide-based formula AA(n),
wherein n is 2-5;
said peptide comprising:
i. an N-terminus amino acid that includes a basic side chain;
ii. an amino acid at any one of positions 2-5 that includes a polar, non-polar, aromatic ring or hetero-atom side-chain that can stabilize a radical species and is configured such that said amino acid at position 2-5 can participate in direct radical transfer from the heme porphyrin of the MPO's active site to said amino acid's side-chain, thus yielding the chemically modified SCPD;
iii. an amino acid at any one of positions 2-5 including a side chain that can interact with the MPO's active site through one or more of ionic, dipolar, pi-pi, hydrophobic or hydrophilic interactions facilitating radical transfer from the heme porphyrin of the MPO's active site to the SCPD; and
iv. an amino acid at any one of positions 2-5 that includes a side chain containing a heteroatom that stabilizes the radical on the chemically modified SCPD;
wherein said chemically modified SCPD is configured to: auto-scavenge by forming a homo dimer via an oxidized linkage with another chemically modified SCPD; or, optionally, to scavenge by forming a hetero dimer via an oxidized linkage with another peptide or protein.

17-36. (canceled)

37. A method of treating a disease or condition in a subject, the method comprising administering to the subject a systems chemico-pharmacology drug (SCPD), whereby the SCPD interacts with a first target that comprises a druggable biomolecular site, whereby the SCPD modifies the properties of the first target and is itself chemically modified, and further whereby the chemically modified SCPD interacts with one or more second targets, thus modifying the each of the second targets' functions;
whereby the disease or condition is treated in the subject.

38-55. (canceled)

56. The method of claim 37, wherein the SCPD comprises a tripeptide KXZ having the formula AA₁-AA₂-AA₃, wherein AA₁ (K) is an amino acid comprising a basic side chain, AA₂ (X) is a polar, non-polar or aromatic amino acid, and AA₃ (Z) is an amino acid possessing a heteroatom that is capable of stabilizing a free radical,
wherein one or more of the second targets is a protein and the protein is HMGB1, RAGE, TRL4, NRf2 or KEAP-1.

57-62. (canceled)

63. A compositional system comprising a systems chemico-pharmacology drug (SCPD) in fluid communication with (a) a first target that comprises a druggable biomolecular site, and (b) a modified first target comprising a druggable biomolecular site that has been modified by the SCPD.

64-84. (canceled)

85. The compositional system of claim 63, further comprising one or more second targets wherein at least one of the one or more second targets is a protein and the protein is HMGB1, RAGE, TRL4, NRf2 or KEAP-1.

86-92. (canceled)
